(19) United States
(12) Patent Application Publication
(10) Pub. No.: US 2024/0076411 A1
(43) Pub. Date: Mar. 7, 2024

(54) MULTISPECIFIC ANTIGEN BINDING PROTEINS

(71) Applicant: WUXI BIOLOGICS IRELAND LIMITED, Dundalk, Co Louth (IE)

(72) Inventors: Xinzhao FAN, Shanghai (CN); Jianqing XU, Shanghai (CN); Yunying CHEN, Shanghai (CN); Xiaofeng LU, Shanghai (CN); Yongqing CHENG, Shanghai (CN); Zhuozhi WANG, Shanghai (CN); Jijie GU, Shanghai (CN)

(21) Appl. No.: 18/270,160

(22) PCT Filed: Dec. 29, 2021
(86) PCT No.: PCT/CN2021/142655

§ 371 Date: Jun. 28, 2023

(30) Foreign Application Priority Data
Dec. 30, 2020 (WO) PCT/CN2020/141675

(51) Int. Cl. C07K 16/46; C07K 16/22; C07K 16/28; C07K 16/30

(52) U.S. Cl. CPC C07K 16/468; C07K 16/22; C07K 16/2818; C07K 16/2878; C07K 16/30; C07K 2317/31; C07K 2317/55; C07K 2317/569; C07K 2317/622; C07K 2317/64; C07K 2317/92; C07K 2317/94

(57) ABSTRACT

This disclosure provides multispecific and multivalent antigen binding proteins. In one aspect, the disclosure provides a multispecific antigen binding protein, comprising a first antigen binding site comprising a first VHH that specifically binds to a first epitope; and a second antigen binding site that specifically binds to a second epitope.

### TECHNICAL FIELD

This disclosure relates to multispecific and multivalent antigen binding proteins, and methods of making and use thereof.

### BACKGROUND

Multispecific antibodies have become increasingly of interest for therapeutic use. While natural antibodies are monospecific, multispecific antibodies (e.g., bispecific antibodies) recognize two or more different epitopes either on the same or on different antigens. With two or more sites for interacting with the target cell, more targeted binding can be achieved. The more targeted binding can lead to the formation of new protein complexes and trigger new cellular contacts. In many cases, additional immune responses can be activated via the redirection of cytotoxic immune effector cells, such as T cells and natural killer (NK) cells, leading to significantly greater targeted cytotoxic effects. The enhancements sometimes can be much greater than if two individual monoclonal antibodies were administered as a combination therapy. The therapeutic use of multispecific antibodies initially focused on effector cell retargeting for cancer therapy. During the past decade, many other therapeutic strategies based on multispecific antibodies have been established. At present, multispecific antibodies have a broad spectrum of use, including e.g., diagnosis, imaging, prophylaxis and therapy.

Despite numerous advantages of multispecific antibodies, developing and manufacturing multispecific antibodies remain to be a challenge. This is largely because conventional methods rely heavily on antigen binding portions of monoclonal antibodies. These antigen binding portions may lose their desirable biochemical and/or biophysical properties in the multispecific antigen binding constructs. In addition, many multispecific antibody formats have mispairing problems, and they are also often associated with aggregation and low expression levels. They are difficult to purify and manufacture. There remains a need for a versatile multispecific antibody platform for drug development and manufacturing.

### SUMMARY

This disclosure relates to multispecific and multivalent antigen binding proteins, and methods of making and use thereof. Provided herein is also a versatile multispecific antibody platform. The antigen-binding portion in the multispecific and multivalent antigen binding proteins can fold properly and retain a high binding affinity with the antigen. In addition, these multispecific and multivalent antigen binding proteins can be easily expressed at a high level and can be readily purified and manufactured. These multispecific antigen binding proteins are particularly suitable for drug development and manufacturing.

In one aspect, the disclosure is related to a multispecific antigen binding protein, comprising (a) an Fc; (b) a first antigen binding site comprising a first single-domain antibody variable domain (VHH) that specifically binds to a first epitope; and (c) a second antigen binding site that specifically binds to a second epitope. In some embodiments, the first antigen binding site and the second antigen binding site are linked to the Fc.

In some embodiments, the first VHH is linked to a CH2 domain in the Fc.

In some embodiments, the first VHH is linked to a CH2 domain in the Fc through a hinge region.

In some embodiments, the first VHH is linked to the C terminal of a CH3 in the Fc.

In some embodiments, the first VHH is linked to a CH1 domain. In some embodiments, the CH1 domain is linked to a CH2 domain in the Fc.

In some embodiments, the first VHH is linked to a VH domain and a CH1 domain, and the CH1 domain is linked to a CH2 domain in the Fc. In some embodiments, the antigen binding protein further comprises a VL domain. In some embodiments, the VH domain and the VL domain associate with each other, forming an antigen binding site.

In some embodiments, the antigen binding protein further comprises a VHH. In some embodiments, the VHH is linked to the VL domain.

In some embodiments, the second antigen-binding site comprises a VH domain and a VL domain. In some embodiments, the VH domain and the VL domain associate with each other, forming the second antigen binding site.

In some embodiments, the antigen binding protein comprises two heavy chains and two light chains.

In some embodiments, the second antigen-binding site comprises a second VHH.

In some embodiments, the second VHH is linked to a CH2 domain in the Fc.

In some embodiments, the second VHH is linked to a CH2 domain in the Fc through a hinge region.

In some embodiments, the second VHH is linked to the C terminal of a CH3 in the Fc.

In some embodiments, the second VHH is linked to a CH1 domain, and the CH1 domain is linked to a CH2 domain in the Fc.

In some embodiments, the second VHH is linked to a VH domain and a CH1 domain. In some embodiments, the CH1 domain is linked to a CH2 domain in the Fc.

In some embodiments, the antigen binding protein further comprises a VH domain and a VL domain. In some embodiments, the VH domain and the VL domain associate with each other, forming an antigen binding site.

In some embodiments, the second VHH is linked to the VH domain. In some embodiments, the second VHH is linked to the VL domain.

In some embodiments, the first epitope and the second epitope are from different antigens. In some embodiments, the first epitope and the second epitope are from the same antigen.

In some embodiments, the antigen binding protein further comprises a third antigen-binding site comprising a third VHH. In some embodiments, the third VHH is linked to the first VHH.

In some embodiments, the third antigen-binding site specifically binds to a third epitope. In some embodiments, the first epitope and the third epitope are from different antigens.

In some embodiments, the third antigen-binding site specifically binds to a third epitope. In some embodiments, the first epitope and the third epitope are from the same antigen.

In some embodiments, the first antigen binding site and the second antigen binding site specifically bind to one or more of the following antigens selected from the group consisting of VEGF, Ang-2, MSLN, GITR, and PD-1.

In some embodiments, the first antigen binding site, the second antigen binding site, and the third antigen binding site specifically bind to one or more of the following antigens selected from the group consisting of VEGF, Ang-2, MSLN, GITR, and PD-1.

In one aspect, the disclosure is related to a multispecific antigen binding protein, comprising (a) a first polypeptide comprising a first single-domain antibody variable domain (VHH1) that specifically binds to a first epitope; and a CH1 domain; (b) a second polypeptide comprising a second single-domain antibody variable domain (VHH2) that specifically binds to a second epitope and a CL domain. In some embodiments, the first polypeptide and the second polypeptide associate with each other to form a dimer through the CH1 domain and the CL domain.

In some embodiments, a VH domain is located between the VHH1 and the CH1 domain, and a VL domain is located between the VHH2 and the CL domain. In some embodiments, the VH and VL associate with each other, forming an antigen binding site.

In some embodiments, the first epitope and the second epitope are from different antigens. In some embodiments, the first epitope and the second epitope are from the same antigen.

In some embodiments, the VHH1 and the VHH2 specifically bind to one or more of the following antigens selected from the group consisting of VEGF, Ang-2, MSLN, GITR, and PD-1.

In one aspect, the disclosure is related to a multispecific antigen binding protein, comprising (a) a first polypeptide comprising a first VHH (VHH1) that specifically binds to a first epitope; and (b) a second polypeptide comprising a second VHH (VHH2) that specifically binds to a second epitope. In some embodiments, the first polypeptide and the second polypeptide associate with each other to form a dimer.

In some embodiments, the first epitope and the second epitope are from the same antigen. In some embodiments, the first epitope and the second epitope are from different antigens.

In some embodiments, the first polypeptide further comprises a first immunoglobulin hinge region, a first CH2 domain and a first CH3 domain. In some embodiments, the second polypeptide further comprises a second immunoglobulin hinge region, a second CH2 domain and a second CH3 domain.

In some embodiments, the VHH1 is linked to the first immunoglobulin hinge region.

In some embodiments, the VHH2 is linked to the second immunoglobulin hinge region.

In some embodiments, the first polypeptide further comprises a first CH1 domain. In some embodiments, the VHH1 is linked to the first CH1 domain.

In some embodiments, the antigen binding protein described herein further comprises a third polypeptide. In some embodiments, the third polypeptide comprises: (a) a third single-domain antibody (VHH3) that specifically binds a third epitope; and (b) a first CL domain. In some embodiments, the first polypeptide and the third polypeptide associate with each other via the interaction between the first CH1 domain and the first CL domain.

In some embodiments, the first epitope and the third epitope are from the same antigen. In some embodiments, the first epitope and the third epitope are from different antigens.

In some embodiments, the second polypeptide further comprises a second CH1 domain.

In some embodiments, the VHH2 is linked to the second CH1 domain.

In some embodiments, the antigen binding protein described herein further comprises a fourth polypeptide which comprises: (a) a fourth VHH (VHH4) that specifically binds a fourth epitope; and (b) a second CL domain. In some embodiments, the second polypeptide and the fourth polypeptide associate with each other via the interaction between the second CH1 domain and the second CL domain.

In some embodiments, the second epitope and the fourth epitope are from the same antigen. In some embodiments, the second epitope and the fourth epitope are from different antigens.

In some embodiments, the first polypeptide further comprises a fifth VHH (VHH5) that specifically binds to a fifth epitope. In some embodiments, the VHH5 is linked to the N-terminus of the first polypeptide.

In some embodiments, the first polypeptide further comprises a fifth VHH (VHH5) that specifically binds to a fifth epitope. In some embodiments, the VHH5 is linked to the C-terminus of the first polypeptide.

In some embodiments, the second polypeptide further comprises a sixth VHH (VHH6) that specifically binds to a sixth epitope. In some embodiments, the VHH6 is linked to the N-terminus of the second polypeptide.

In some embodiments, the second polypeptide further comprises a sixth VHH (VHH6) that specifically binds to a sixth epitope. In some embodiments, the VHH6 is linked to the C-terminus of the second polypeptide.

In some embodiments, the antigen binding protein comprises a third polypeptide. In some embodiments, the third polypeptide further comprises a seventh VHH (VHH7) that specifically binds to a seventh epitope. In some embodiments, the VHH7 is linked to the N-terminus of the third polypeptide.

In some embodiments, the antigen binding protein comprises a third polypeptide. In some embodiments, the third polypeptide further comprises a seventh VHH (VHH7) that specifically binds to a seventh epitope. In some embodiments, the VHH7 is linked to the C-terminus of the third polypeptide.

In some embodiments, the antigen binding protein comprises a fourth polypeptide. In some embodiments, the fourth polypeptide further comprises an eighth VHH (VHH8) that specifically binds to an eighth epitope. In some embodiments, the VHH8 is linked to the N-terminus of the fourth polypeptide.

In some embodiments, the antigen binding protein comprises a fourth polypeptide. In some embodiments, the fourth polypeptide further comprises an eighth VHH (VHH8) that specifically binds to an eighth epitope. In some embodiments, the VHH8 is linked to the C-terminus of the fourth polypeptide.

In one aspect, the disclosure is related to an antigen binding protein, comprising (a) a first polypeptide comprising a first VHH (VHH1) that specifically binds to a first epitope; and (b) a second polypeptide comprising a first heavy chain variable domain (VH1) and a first CH1 domain of a first Fab domain. In some embodiments, the first Fab domain specifically binds to a second epitope. In some embodiments, the first polypeptide and the second polypeptide associate with each other to form a dimer.

In some embodiments, the first epitope and the second epitope are from the same antigen. In some embodiments, the first epitope and the second epitope are from different antigens.

In some embodiments, the first polypeptide further comprises from N-terminus to C-terminus: a first immunoglobulin hinge region, a first CH2 domain and a first CH3 domain. In some embodiments, the VHH1 is linked to the first immunoglobulin hinge region.

In some embodiments, the first polypeptide further comprises from N-terminus to C-terminus: a second heavy chain variable domain VH (VH2) and a second CH1 domain of a second Fab domain, a first immunoglobulin hinge region, a first CH2 domain and a first CH3 domain.

In some embodiments, the VHH1 is linked to N-terminus of the VH2.

In some embodiments, the VHH1 is located between the second CH1 domain and the first immunoglobulin hinge region.

In some embodiments, the antigen binding protein described herein further comprises a second VHH (VHH2). In some embodiments, the VHH2 is linked to a second light chain variable domain (VL2) of the second Fab domain.

In some embodiments, the second polypeptide further comprises from N-terminus to C-terminus: a second immunoglobulin hinge region; a second CH2 domain; and a second CH3 domain.

In some embodiments, the antigen binding protein described herein further comprises a third VHH (VHH3). In some embodiments, the VHH3 is linked to the N-terminus of the VH1.

In some embodiments, the antigen binding protein described herein further comprises a third VHH (VHH3). In some embodiments, the VHH3 is located between the first CH1 domain and the second immunoglobulin hinge region.

In some embodiments, the antigen binding protein described herein, further comprising a fourth VHH (VHH4). In some embodiments, the VHH4 is linked to a first light chain variable domain (VL1) of the first Fab domain.

In some embodiments, the first polypeptide further comprises a fifth VHH (VHH5) that specifically binds to a fifth epitope. In some embodiments, the VHH5 is linked to the N-terminus of the first polypeptide.

In some embodiments, the first polypeptide further comprises a fifth VHH (VHH5) that specifically binds to a fifth epitope. In some embodiments, the VHH5 is linked to the C-terminus of the first polypeptide.

In some embodiments, the second polypeptide further comprises a sixth VHH (VHH6) that specifically binds to a sixth epitope. In some embodiments, the VHH6 is linked to the N-terminus of the second polypeptide.

In some embodiments, the second polypeptide further comprises a sixth VHH (VHH6) that specifically binds to a sixth epitope. In some embodiments, the VHH6 is linked to the C-terminus of the second polypeptide.

In some embodiments, the VHH1, VHH2, VHH3, VHH4, VHH5, VHH6, VHH7, and/or VHH8 specifically bind to a cancer associated antigen or a cancer specific antigen.

In some embodiments, the VHH1, VHH2, VHH3, VHH4, VHH5, VHH6, VHH7, and/or VHH8 specifically bind to an antigen. In some embodiments, the antigen is selected from the group consisting of VEGF, Ang2, Mesothelin, GITR, HER2, BRAF, EGFR, VEGFR2, CD20, RANKL, CD38, and CD52.

In some embodiments, the VHH1, VHH2, VHH3, VHH4, VHH5, VHH6, VHH7, and/or VHH8 specifically bind to VEGF, Ang2, Mesothelin, or GITR.

In some embodiments, the VHH1, VHH2, VHH3, VHH4, VHH5, VHH6, VHH7, and/or VHH8 specifically bind to an immune checkpoint molecule.

In some embodiments, the immune checkpoint molecule is selected from the group consisting of PD-1, PD-L1, PD-L2, CTLA-4, B7-H3, TIM-3, LAG-3, VISTA, ICOS, 4-1BB, OX40, GITR, and CD40.

In some embodiments, the immune checkpoint molecule is PD-1.

In some embodiments, the antigen binding protein specifically binds to at least 1, at least 2, at least 3, at least 4, at least 5, at least 6, at least 7, or at least 8 epitopes.

In one aspect, the disclosure is related to an antigen binding protein, comprising one or more of the following antigen binding sites:
(a) an antigen binding site targeting vascular endothelial growth factor (VEGF);
(b) an antigen binding site targeting angiopoietin-2 (Ang-2);
(c) an antigen binding site targeting programmed cell death protein 1 (PD-1); and/or
(d) an antigen binding site targeting Mesothelin (MSLN); and/or
(e) an antigen binding site targeting Glucocorticoid-Induced TNFR-Related Protein (GITR).

In some embodiments, the antigen binding protein described herein comprises an antigen binding site targeting VEGF and an antigen binding site targeting Ang-2.

In some embodiments, the antigen binding protein described herein comprises an antigen binding site targeting VEGF and an antigen binding site targeting PD-1.

In some embodiments, the antigen binding protein described herein comprises an antigen binding site targeting Ang-2 and an antigen binding site targeting PD-1.

In some embodiments, the antigen binding protein described herein comprises an antigen binding site targeting VEGF, an antigen binding site targeting Ang-2, and an antigen binding site targeting PD-1.

In some embodiments, the antigen binding protein comprises at least one, at least two, at least three, or at least four antigen binding sites that target VEGF.

In some embodiments, the antigen binding protein comprises at least one, at least two, at least three, or at least four antigen binding sites that target Ang-2.

In some embodiments, the antigen binding protein comprises at least one, at least two, at least three, or at least four antigen binding sites that target MSLN.

In some embodiments, the antigen binding protein comprises at least one, at least two, at least three, or at least four antigen binding sites that target GITR.

In some embodiments, the antigen binding protein comprises at least one, at least two, at least three, or at least four antigen binding sites that target PD-1.

In some embodiments, one or more antigen binding sites comprise a heavy chain variable domain (VH) and a light chain variable domain (VL).

In some embodiments, one or more antigen binding sites comprise a VHH.

In some embodiments, the antigen binding protein can be produced at an expression level of at least 5 mg/L, at least 6 mg/L, at least 7 mg/L, at least 8 mg/L, at least 9 mg/L, at least 10 mg/L, at least 20 mg/L, at least 30 mg/L, at least 40 mg/L, at least 50 mg/L, at least 60 mg/L, at least 70 mg/L, at least 80 mg/L, at least 90 mg/L, at least 100 mg/L, at least at least 110 mg/L, at least 120 mg/L, at least 130 mg/L, at least 140 mg/L, at least 150 mg/L, at least 160 mg/L, at least 170 mg/L, at least 180 mg/L, at least 190 mg/L, or at least 200 mg/L.

In some embodiments, the antigen binding protein can be produced with a purify of at least 70%, at least 75%, at least 80%, at least 85, at least 90%, at least 91%, at least 92%, at least 93%, at least 94%, at least 95%, at least 96%, at least 97%, at least 98%, at least 99%, or about 100% (e.g., after being purified by Protein A chromatography).

In some embodiments, the antigen binding protein has a Tm of at least 57° C., at least 58° C., at least 59° C., at least 60° C., at least 61° C., at least 62° C., at least 63° C., at least 64° C., or at least 65° C.

In some embodiments, the antigen binding protein has an EC50 value for binding to VEGF, Ang-2, MSLN, PD-1, or GITR of less than 0.01 μg/ml, less than 0.02 μg/ml, less than 0.03 μg/ml, less than 0.04 μg/ml, less than 0.05 μg/ml, less than 0.06 μg/ml, less than 0.07 μg/ml, less than 0.08 μg/ml, less than 0.09 μg/ml, less than 0.10 μg/ml, less than 0.11 μg/ml, less than 0.12 μg/ml, less than 0.13 μg/ml, less than 0.14 μg/ml, less than 0.15 μg/ml, less than 0.16 μg/ml, less than 0.17 μg/ml, less than 0.18 μg/ml, less than 0.19 μg/ml, less than 0.20 μg/ml, less than 0.21 μg/ml, less than 0.22 μg/ml, less than 0.23 μg/ml, less than 0.24 μg/ml, less than 0.25 μg/ml, or less than 0.30 μg/ml.

In some embodiments, the antigen binding protein has a binding affinity of at least 80%, 85%, 90%, 95%, or 100% of the binding affinity of a parental antibody targeting the same target.

In one aspect, the disclosure is related to a method of treating a subject having cancer, the method comprising administering a therapeutically effective amount of a composition comprising the antigen binding protein as described herein to the subject.

In some embodiments, the subject has a VEGF-expressing, Ang-2-expressing, and/or MSLN-expressing cancer.

In some embodiments, the cancer is selected from the group consisting of breast cancer, renal cancer, melanoma, lung cancer, glioblastoma, head and neck cancer, prostate cancer, ovarian carcinoma, bladder carcinoma, and lymphoma.

In one aspect, the disclosure is related to a method of treating a subject having an autoimmune disease or an inflammatory disease, the method comprising administering a therapeutically effective amount of a composition comprising the antigen binding protein as described herein to the subject.

In one aspect, the disclosure is related to an antibody-drug conjugate comprising the antigen binding protein as described herein, covalently bound to a therapeutic agent.

In some embodiments, the therapeutic agent is a cytotoxic or cytostatic agent.

In one aspect, the disclosure is related to a pharmaceutical composition comprising the antigen binding protein as described herein and a pharmaceutically acceptor carrier.

In one aspect, the disclosure is related to a pharmaceutical composition comprising the antibody-drug conjugate as described herein, and a pharmaceutically acceptable carrier.

In one aspect, the disclosure is related to a nucleic acid encoding the antigen binding protein as described herein.

In one aspect, the disclosure is related to a vector comprising the nucleic acid as described herein.

In one aspect, the disclosure is related to a host cell comprising the nucleic acid as described herein or the vector as described herein.

In one aspect, the disclosure is related to a method for producing an antigen binding protein, the method comprising culturing the host cell as described herein under conditions suitable to produce the antigen binding protein.

As used herein, the term “antigen binding protein” or “antigen binding construct” refers to a protein that contains at least one antigen binding site that is capable of specifically binding to an antigen. An antigen binding protein can have one, two, three, four, five, six, seven, eight, nine, ten, or more than ten polypeptides. It can have one, two, three, four, five, six, seven, eight, nine, ten, or more than ten antigen binding sites. In some embodiments, an antigen binding protein can be any antibody as described herein.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope in an antigen. Non-limiting examples of antibodies include: monoclonal antibodies, polyclonal antibodies, multi-specific antibodies (e.g., bi-specific antibodies), single-chain antibodies, single variable domain (VHH) antibodies, chimeric antibodies, human antibodies, and humanized antibodies. In some embodiments, an antibody can contain an Fc region of a human antibody. The term antibody also includes derivatives, e.g., multi-specific antibodies, bi-specific antibodies, single-chain antibodies, diabodies, and linear antibodies formed from these antibodies or antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain (VH), a variable domain of light chain (VL), or a VHH). Non-limiting examples of antibody fragments include, e.g., Fab, Fab′, F(ab′)2, and Fv fragments, ScFv, and VHH.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antigen binding protein, an antibody or an antigen-binding fragment, the phrases “specifically binding” and “specifically binds” mean that the antibody or an antigen-binding fragment interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to PD-1 may be referred to as a PD1-specific antibody or an anti-PD1 antibody.

As used herein, the term “bispecific antibody” refers to an antibody that binds to two different epitopes. The epitopes can be on the same antigen or on different antigens.

As used herein, the term “trispecific antibody” refers to an antibody that binds to three different epitopes. The epitopes can be on the same antigen or on different antigens.

As used herein, the term “multispecific antibody” refers to an antibody that binds to two or more different epitopes. The epitopes can be on the same antigen or on different antigens. A multispecific antibody can be e.g., a bispecific antibody or a trispecific antibody. In some embodiments, the multispecific antibody binds to two, three, four, five, six, seven, eight, nine, ten, or more than ten different epitopes.

As used herein, a “VHH” refers to an antibody variable domain that can specifically binds to an antigen. They are variable domains of heavy chain antibodies or single-domain antibodies (nanobody). In some embodiments, the VHH is a humanized VHH. As the VHH by itself can bind to an antigen, the VHH is also known as single-domain antibody (sdAb) or nanobody. The VHH can be obtained e.g., from immunization of dromedaries, camels, llamas, alpacas or shark, from phage display library, or from antibody engineering.

As used herein, the term “valent” refers to a specified number of binding sites in an antigen binding protein or an antibody molecule. For example, a natural antibody or a full length antibody has two binding sites and is bivalent. As such, the terms “trivalent”, “tetravalent”, “pentavalent” and “hexavalent” denote the presence of two binding sites, three binding sites, four binding sites, five binding sites, and six binding sites, respectively, in an antibody or an antigen binding protein.

As used herein, “Fc region” refers to the fragment crystallizable region of an antibody. The Fc region can be a native sequence Fc region or an altered Fc region. Fc can be derived from various immunoglobulins, including e.g., IgG1, IgG2, IgG3, IgG4, and other classes such as IgA, IgD, IgE and IgM. The Fc region of an immunoglobulin generally comprises a CH2 domain and a CH3 domain, and optionally comprises a CH4 domain.

As used herein, “link” means a polypeptide is linked to another polypeptide through one or more covalent bonds. In some embodiments, a polypeptide is linked to another polypeptide by a peptide bond or a disulfide bond. In some embodiments, a polypeptide is linked to another polypeptide through a peptide linker sequence. The peptide linker sequence can have one or more amino acids. In some embodiments, the two polypeptides can be linked directly through a peptide bond without any peptide linker sequences between them. In some embodiments, a polypeptide can be linked to another polypeptide by fusion, forming a fusion polypeptide. In many cases, fusion polypeptides are created through the joining of two or more nucleic acid sequences encoding these polypeptides. In some embodiments, a polypeptide is directly fused to another polypeptide without any peptide linker sequence. In some embodiments, a polypeptide is fused to another polypeptide with a peptide linker sequence.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a table showing schematic diagrams of structural elements of the multispecific antigen binding proteins described in the disclosure.

FIG. 1B is a schematic structure of W366001-T1U1.F82-1.uIgG4V1 (or “F82”).

FIG. 1C is a schematic structure of W366001-T1U1.F83-1.uIgG4V1 (or “F83”).

FIG. 1D is a schematic structure of W366001-U1T1.F84-1.uIgG4V1 (or “F84”).

FIG. 1E is a schematic structure of W366001-U1T1.G1-1.uIgG4V1 (or “G1”).

FIG. 1F is a schematic structure of W366001-U1T1.G32-1.uIgG4V1 (or “G32”).

FIG. 1G is a schematic structure of W366001-U1T1.G33-1.uIgG4V1 (or “G33”).

FIG. 1H is a schematic structure of W366001-U1T1.H9-1.uIgG4V1 (or “H9”).

FIG. 2A shows gel electrophoresis results of F82.

FIG. 2B shows SEC result of F82.

FIG. 2C shows melting curve of F82.

FIG. 2D shows VEGF binding results of F82, W366001-cAb1, and W366001-cAb2. NC is negative control. W366001-cAb1 (monovalent version) and W366001-cAb2 (bivalent version) are parental antibodies targeting VEGF.

FIG. 2E shows Ang-2 binding results of F82, W366001-cAb3, and W366001-cAb4. NC is negative control. W366001-cAb3 (monovalent version) and W366001-cAb4 (bivalent version) are parental antibodies targeting Ang-2.

FIG. 3A shows gel electrophoresis results of F83.

FIG. 3B shows SEC result of F83.

FIG. 3C shows melting curve of F83.

FIG. 3D shows VEGF binding results of F83, W366001-cAb1, and W366001-cAb2. NC is negative control. W366001-cAb1 (monovalent version) and W366001-cAb2 (bivalent version) are parental antibodies targeting VEGF.

FIG. 3E shows Ang-2 binding results of F83, W366001-cAb3, and W366001-cAb4. NC is negative control. W366001-cAb3 (monovalent version) and W366001-cAb4 (bivalent version) are parental antibodies targeting Ang-2.

FIG. 4A shows gel electrophoresis results of F84.

FIG. 4B shows SEC result of F84.

FIG. 4C shows melting curve of F84.

FIG. 4D shows VEGF binding results of F84, W366001-cAb1, and W366001-cAb2. NC is negative control. W366001-cAb1 (monovalent version) and W366001-cAb2 (bivalent version) are parental antibodies targeting VEGF.

FIG. 4E shows Ang-2 binding results of F84, W366001-cAb3, and W366001-cAb4. NC is negative control. W366001-cAb3 (monovalent version) and W366001-cAb4 (bivalent version) are parental antibodies targeting Ang-2.

FIG. 5A shows gel electrophoresis results of G1.

FIG. 5B shows SEC result of G1.

FIG. 5C shows melting curve of G1.

FIG. 5D shows VEGF binding results of G1, W366001-cAb1, and W366001-cAb2. NC is negative control. W366001-cAb1 (monovalent version) and W366001-cAb2 (bivalent version) are parental antibodies targeting VEGF.

FIG. 5E shows Ang-2 binding results of G1, W366001-cAb3, and W366001-cAb4. NC is negative control. W366001-cAb3 (monovalent version) and W366001-cAb4 (bivalent version) are parental antibodies targeting Ang-2.

FIG. 6A shows gel electrophoresis results of G32.

FIG. 6B shows SEC result of G32.

FIG. 6C shows melting curve of G32.

FIG. 6D shows VEGF binding results of G32, W366001-cAb1, and W366001-cAb2. NC is negative control. W366001-cAb1 (monovalent version) and W366001-cAb2 (bivalent version) are parental antibodies targeting VEGF.

FIG. 6E shows Ang-2 binding results of G32, W366001-cAb3, and W366001-cAb4. NC is negative control. W366001-cAb3 (monovalent version) and W366001-cAb4 (bivalent version) are parental antibodies targeting Ang-2.

FIG. 7A shows gel electrophoresis results of G33.

FIG. 7B shows SEC result of G33.

FIG. 7C shows melting curve of G33.

FIG. 7D shows VEGF binding results of G33, W366001-cAb1, and W366001-cAb2. NC is negative control. W366001-cAb1 (monovalent version) and W366001-cAb2 (bivalent version) are parental antibodies targeting VEGF.

FIG. 7E shows Ang-2 binding results of G33, W366001-cAb3, and W366001-cAb4. NC is negative control. W366001-cAb3 (monovalent version) and W366001-cAb4 (bivalent version) are parental antibodies targeting Ang-2.

FIG. 8A shows gel electrophoresis results of H9. Lane 1 is the supernatant sample before purification.

FIG. 8B shows SEC result of H9.

FIG. 8C shows melting curve of H9.

FIG. 8D shows VEGF binding results of H9, W366001-cAb1, and W366001-cAb2. NC is negative control. W366001-cAb1 (monovalent version) and W366001-cAb2 (bivalent version) are parental antibodies targeting VEGF.

FIG. 8E shows Ang-2 binding results of H9, W366001-cAb3, and W366001-cAb4. NC is negative control. W366001-cAb3 (monovalent version) and W366001-cAb4 (bivalent version) are parental antibodies targeting Ang-2.

FIG. 9A is a schematic structure of W366001-T1U1.E32-1.uIgG4V1 (or “E32”).

FIG. 9B is a schematic structure of W366001-U1T1.G44-1.uIgG4V1 (or “G44”).

FIG. 9C is a schematic structure of W366001-U1T1.G45-1.uIgG4V1 (or “G45”).

FIG. 9D is a schematic structure of W366001-U1T1.G46-1.uIgG4V1 (or “G46”).

FIG. 9E is a schematic structure of W366001-U1T1.H14-1.uIgG4V1 (or “H14”).

FIG. 9F is a schematic structure of W366001-U1T1.H6-1.uIgG4V1 (or “H6”).

FIG. 10A shows gel electrophoresis results of E32. M is a protein marker.

FIG. 10B shows SEC result of E32.

FIG. 10C shows melting curve of E32.

FIG. 10D shows VEGF binding results of E32, W366001-cAb1, and W366001-cAb2. NC is negative control. W366001-cAb1 (monovalent version) and W366001-cAb2 (bivalent version) are parental antibodies targeting VEGF.

FIG. 10E shows Ang-2 binding results of E32, W366001-cAb3, and W366001-cAb4. NC is negative control. W366001-cAb3 (monovalent version) and W366001-cAb4 (bivalent version) are parental antibodies targeting Ang-2.

FIG. 11A shows gel electrophoresis results of G44.

FIG. 11B shows SEC result of G44.

FIG. 11C shows melting curve of G44.

FIG. 11D shows VEGF binding results of G44, W366001-cAb1, and W366001-cAb2. NC is negative control. W366001-cAb1 (monovalent version) and W366001-cAb2 (bivalent version) are parental antibodies targeting VEGF.

FIG. 11E shows Ang-2 binding results of G44, W366001-cAb3, and W366001-cAb4. NC is negative control. W366001-cAb3 (monovalent version) and W366001-cAb4 (bivalent version) are parental antibodies targeting Ang-2.

FIG. 12A shows gel electrophoresis results of G45.

FIG. 12B shows SEC result of G45.

FIG. 12C shows melting curve of G45.

FIG. 12D shows VEGF binding results of G45, W366001-cAb1, and W366001-cAb2. NC is negative control. W366001-cAb1 (monovalent version) and W366001-cAb2 (bivalent version) are parental antibodies targeting VEGF.

FIG. 12E shows Ang-2 binding results of G45, W366001-cAb3, and W366001-cAb4. NC is negative control. W366001-cAb3 (monovalent version) and W366001-cAb4 (bivalent version) are parental antibodies targeting Ang-2.

FIG. 13A shows gel electrophoresis results of G46.

FIG. 13B shows SEC result of G46.

FIG. 13C shows melting curve of G46.

FIG. 13D shows VEGF binding results of G46, W366001-cAb1, and W366001-cAb2. NC is negative control. W366001-cAb1 (monovalent version) and W366001-cAb2 (bivalent version) are parental antibodies targeting VEGF.

FIG. 13E shows Ang-2 binding results of G46, W366001-cAb3, and W366001-cAb4. NC is negative control. W366001-cAb3 (monovalent version) and W366001-cAb4 (bivalent version) are parental antibodies targeting Ang-2.

FIG. 14A shows gel electrophoresis results of H14.

FIG. 14B shows SEC result of H14.

FIG. 14C shows melting curve of H14.

FIG. 14D shows VEGF binding results of H14, W366001-cAb1, and W366001-cAb2. NC is negative control. W366001-cAb1 (monovalent version) and W366001-cAb2 (bivalent version) are parental antibodies targeting VEGF.

FIG. 14E shows Ang-2 binding results of H14, W366001-cAb3, and W366001-cAb4. NC is negative control. W366001-cAb3 (monovalent version) and W366001-cAb4 (bivalent version) are parental antibodies targeting Ang-2.

FIG. 15A shows gel electrophoresis results of H6. The lane between the two lanes labeled with M and N is the supernatant sample before purification.

FIG. 15B shows SEC result of H6.

FIG. 15C shows melting curve of H6.

FIG. 15D shows VEGF binding results of H6, W366001-cAb1, and W366001-cAb2. NC is negative control. W366001-cAb1 (monovalent version) and W366001-cAb2 (bivalent version) are parental antibodies targeting VEGF.

FIG. 15E shows Ang-2 binding results of H6, W366001-cAb3, and W366001-cAb4. NC is negative control. W366001-cAb3 (monovalent version) and W366001-cAb4 (bivalent version) are parental antibodies targeting Ang-2.

FIG. 16A is a schematic structure of W366002-U12T1.E28-1.uIgG4V1 (or “E28”).

FIG. 16B is a schematic structure of W366002-T1U12.F43-1.uIgG4V1 (or “F43”).

FIG. 16C is a schematic structure of W366002-U12T1.F85R-1.uIgG4V1 (or F85R).

FIG. 16D is a schematic structure of W366002-U12T1.F45R-1.uIgG4V1 (or “F45R”).

FIG. 16E is a schematic structure of W366002-U12T1.G58-1.uIgG4V1 (or “G58”).

FIG. 16F is a schematic structure of W366002-T1U12.H27-1.uIgG4V1 (or “H27”).

FIG. 16G is a schematic structure of W366002-T1U12.H22-1.uIgG4V1 (or “H22”).

FIG. 16H is a schematic structure of W366002-T1U12.G47-1.uIgG4V1 (or “G47”).

FIG. 17A shows gel electrophoresis results of E28.

FIG. 17B shows SEC result of E28.

FIG. 17C shows melting curve of E28.

FIG. 17D shows VEGF binding results of E28, W366001-cAb1, and W366001-cAb2. NC is negative control. W366001-cAb1 (monovalent version) and W366001-cAb2 (bivalent version) are parental antibodies targeting VEGF.

FIG. 17E shows PD-1 binding results of E28, W366001-cAb11, and W366001-cAb12. NC is negative control. W366001-cAb11 (monovalent version) and W366001-cAb12 (bivalent version) are parental antibodies targeting PD-1.

FIG. 18A shows gel electrophoresis results of F43.

FIG. 18B shows SEC result of F43.

FIG. 18C shows melting curve of F43.

FIG. 18D shows VEGF binding results of F43, W366001-cAb1, and W366001-cAb2. NC is negative control. W366001-cAb1 (monovalent version) and W366001-cAb2 (bivalent version) are parental antibodies targeting VEGF.

FIG. 18E shows PD-1 binding results of F43, W366001-cAb11, and W366001-cAb12. NC is negative control. W366001-cAb11 (monovalent version) and W366001-cAb12 (bivalent version) are parental antibodies targeting PD-1.

FIG. 19A shows gel electrophoresis results of F85R.

FIG. 19B shows SEC result of F85R.

FIG. 19C shows melting curve of F85R.

FIG. 19D shows VEGF binding results of F85R, W366001-cAb1, and W366001-cAb2. NC is negative control. W366001-cAb1 (monovalent version) and W366001-cAb2 (bivalent version) are parental antibodies targeting VEGF.

FIG. 19E shows PD-1 binding results of F85R, W366001-cAb11, and W366001-cAb12. NC is negative control. W366001-cAb11 (monovalent version) and W366001-cAb12 (bivalent version) are parental antibodies targeting PD-1.

FIG. 20A shows gel electrophoresis results of F45R.

FIG. 20B shows SEC result of F45R.

FIG. 20C shows melting curve of F45R.

FIG. 20D shows VEGF binding results of F45R, W366001-cAb1, and W366001-cAb2. NC is negative control. W366001-cAb1 (monovalent version) and W366001-cAb2 (bivalent version) are parental antibodies targeting VEGF.

FIG. 20E shows PD-1 binding results of F45R, W366001-cAb11, and W366001-cAb12. NC is negative control. W366001-cAb11 (monovalent version) and W366001-cAb12 (bivalent version) are parental antibodies targeting PD-1.

FIG. 21A shows gel electrophoresis results of G58.

FIG. 21B shows SEC result of G58.

FIG. 21C shows melting curve of G58.

FIG. 21D shows VEGF binding results of G58, W366001-cAb1, and W366001-cAb2. NC is negative control. W366001-cAb1 (monovalent version) and W366001-cAb2 (bivalent version) are parental antibodies targeting VEGF.

FIG. 21E shows PD-1 binding results of G58, W366001-cAb11, and W366001-cAb12. NC is negative control. W366001-cAb11 (monovalent version) and W366001-cAb12 (bivalent version) are parental antibodies targeting PD-1.

FIG. 22A shows gel electrophoresis results of H27.

FIG. 22B shows SEC result of H27.

FIG. 22C shows melting curve of H27.

FIG. 22D shows VEGF binding results of H27, W366001-cAb1, and W366001-cAb2. NC is negative control. W366001-cAb1 (monovalent version) and W366001-cAb2 (bivalent version) are parental antibodies targeting VEGF.

FIG. 22E shows PD-1 binding results of H27, W366001-cAb11, and W366001-cAb12. NC is negative control. W366001-cAb11 (monovalent version) and W366001-cAb12 (bivalent version) are parental antibodies targeting PD-1.

FIG. 23A shows gel electrophoresis results of H22.

FIG. 23B shows SEC result of H22.

FIG. 23C shows melting curve of H22.

FIG. 23D shows VEGF binding results of H22, W366001-cAb1, and W366001-cAb2. NC is negative control. W366001-cAb1 (monovalent version) and W366001-cAb2 (bivalent version) are parental antibodies targeting VEGF.

FIG. 23E shows PD-1 binding results of H22, W366001-cAb11, and W366001-cAb12. NC is negative control. W366001-cAb11 (monovalent version) and W366001-cAb12 (bivalent version) are parental antibodies targeting PD-1.

FIG. 24A shows gel electrophoresis results of G47.

FIG. 24B shows SEC result of G47.

FIG. 24C shows melting curve of G47.

FIG. 24D shows VEGF binding results of G47, W366001-cAb1, and W366001-cAb2. NC is negative control. W366001-cAb1 (monovalent version) and W366001-cAb2 (bivalent version) are parental antibodies targeting VEGF.

FIG. 24E shows PD-1 binding results of G47, W366001-cAb11, and W366001-cAb12. NC is negative control. W366001-cAb11 (monovalent version) and W366001-cAb12 (bivalent version) are parental antibodies targeting PD-1.

FIG. 25A is a schematic structure of W366003-T1U1W1123-1.uIgG4V1 (or “123”).

FIG. 25B is a schematic structure of W366003-T1U1W1.L52-1.uIgG4V1 (or “L52”).

FIG. 25C is a schematic structure of W366003-T1U1W3.L1-1.uIgG4V1 (or “L1”).

FIG. 25D is a schematic structure of W366003-T1W1U1.H27-1.uIgG4V1 (or “H27-1”).

FIG. 25E is a schematic structure of W366003-T1U1W3.L54-1.uIgG4V1 (or “L54”).

FIG. 25F is a schematic structure of W366003-T1U1W3.L55-1.uIgG4V1 (or “L55”).

FIG. 25G is a schematic structure of W366003-T1U1W3.L56-1.uIgG4V1 (or “L56”).

FIG. 25H is a schematic structure of W366003-T1U1W1.L51-1.uIgG4V1 (or “L51”).

FIG. 25I is a schematic structure of W366003-T1U1W1.L57-1.uIgG4V1 (or “L57”).

FIG. 25J is a schematic structure of W366003-T1U1W1.L58-1.uIgG4V1 (or “L58”).

FIG. 26A shows gel electrophoresis results of 123.

FIG. 26B shows SEC result of 123.

FIG. 26C shows melting curve of 123.

FIG. 26D shows VEGF binding results of 123, W366001-cAb1, and W366001-cAb2. NC is negative control. W366001-cAb1 (monovalent version) and W366001-cAb2 (bivalent version) are parental antibodies targeting VEGF.

FIG. 26E shows Ang-2 binding results of 123, W366001-cAb3, and W366001-cAb4. NC is negative control. W366001-cAb3 (monovalent version) and W366001-cAb4 (bivalent version) are parental antibodies targeting Ang-2.

FIG. 26F shows PD-1 binding results of 123, W366001-cAb11, and W366001-cAb12. NC is negative control. W366001-cAb11 (monovalent version) and W366001-cAb12 (bivalent version) are parental antibodies targeting PD-1.

FIG. 27A shows gel electrophoresis results of L52.

FIG. 27B shows SEC result of L52.

FIG. 27C shows melting curve of L52.

FIG. 27D shows VEGF binding results of L52, W366001-cAb1, and W366001-cAb2. NC is negative control. W366001-cAb1 (monovalent version) and W366001-cAb2 (bivalent version) are parental antibodies targeting VEGF.

FIG. 27E shows Ang-2 binding results of L52, W366001-cAb3, and W366001-cAb4. NC is negative control. W366001-cAb3 (monovalent version) and W366001-cAb4 (bivalent version) are parental antibodies targeting Ang-2.

FIG. 27F shows PD-1 binding results of L52, W366001-cAb11, and W366001-cAb12. NC is negative control. W366001-cAb11 (monovalent version) and W366001-cAb12 (bivalent version) are parental antibodies targeting PD-1.

FIG. 28A shows gel electrophoresis results of L1.

FIG. 28B shows SEC result of L1.

FIG. 28C shows melting curve of L1.

FIG. 28D shows VEGF binding results of L1, W366001-cAb1, and W366001-cAb2. NC is negative control. W366001-cAb1 (monovalent version) and W366001-cAb2 (bivalent version) are parental antibodies targeting VEGF.

FIG. 28E shows Ang-2 binding results of L1, W366001-cAb3, and W366001-cAb4. NC is negative control. W366001-cAb3 (monovalent version) and W366001-cAb4 (bivalent version) are parental antibodies targeting Ang-2.

FIG. 28F shows MSLN binding results of L1, W366001-cAb5, and W366001-cAb6. NC is negative control. W366001-cAb5 (monovalent version) and W366001-cAb6 (bivalent version) are parental antibodies targeting MSLN.

FIG. 29A shows gel electrophoresis results of H27-1.

FIG. 29B shows SEC result of H27-1.

FIG. 29C shows melting curve of H27-1.

FIG. 29D shows VEGF binding results of H27-1, W366001-cAb1, and W366001-cAb2. NC is negative control. W366001-cAb1 (monovalent version) and W366001-cAb2 (bivalent version) are parental antibodies targeting VEGF.

FIG. 29E shows Ang-2 binding results of H27-1, W366001-cAb3, and W366001-cAb4. NC is negative control. W366001-cAb3 (monovalent version) and W366001-cAb4 (bivalent version) are parental antibodies targeting Ang-2.

FIG. 29F shows PD-1 binding results of H27-1, W366001-cAb11, and W366001-cAb12. NC is negative control. W366001-cAb11 (monovalent version) and W366001-cAb12 (bivalent version) are parental antibodies targeting PD-1.

FIG. 30A shows gel electrophoresis results of L54.

FIG. 30B shows SEC result of L54.

FIG. 30C shows melting curve of L54.

FIG. 30D shows VEGF binding results of L54, W366001-cAb1, and W366001-cAb2. NC is negative control. W366001-cAb1 (monovalent version) and W366001-cAb2 (bivalent version) are parental antibodies targeting VEGF.

FIG. 30E shows Ang-2 binding results of L54, W366001-cAb3, and W366001-cAb4. NC are parental antibodies targeting Ang-2.

FIG. 30F shows MSLN binding results of L54, W366001-cAb5, and W366001-cAb6. NC is negative control. W366001-cAb5 (monovalent version) and W366001-cAb6 (bivalent version) are parental antibodies targeting MSLN.

FIG. 31A shows gel electrophoresis results of L55.

FIG. 31B shows SEC result of L55.

FIG. 31C shows melting curve of L55.

FIG. 31D shows VEGF binding results of L55, W366001-cAb1, and W366001-cAb2. NC is negative control. W366001-cAb1 (monovalent version) and W366001-cAb2 (bivalent version) are parental antibodies targeting VEGF.

FIG. 31E shows Ang-2 binding results of L55, W366001-cAb3, and W366001-cAb4. NC is negative control. W366001-cAb3 (monovalent version) and W366001-cAb4 (bivalent version) are parental antibodies targeting Ang-2.

FIG. 31F shows MSLN binding results of L55, W366001-cAb5, and W366001-cAb6. NC is negative control. W366001-cAb5 (monovalent version) and W366001-cAb6 (bivalent version) are parental antibodies targeting MSLN.

FIG. 32A shows gel electrophoresis results of L56.

FIG. 32B shows SEC result of L56.

FIG. 32C shows melting curve of L56.

FIG. 32D shows VEGF binding results of L56, W366001-cAb1, and W366001-cAb2. NC is negative control. W366001-cAb1 (monovalent version) and W366001-cAb2 (bivalent version) are parental antibodies targeting VEGF.

FIG. 32E shows Ang-2 binding results of L56, W366001-cAb3, and W366001-cAb4. NC is negative control. W366001-cAb3 (monovalent version) and W366001-cAb4 (bivalent version) are parental antibodies targeting Ang-2.

FIG. 32F shows MSLN binding results of L56, W366001-cAb5, and W366001-cAb6. NC is negative control. W366001-cAb5 (monovalent version) and W366001-cAb6 (bivalent version) are parental antibodies targeting MSLN.

FIG. 33A shows gel electrophoresis results of L51.

FIG. 33B shows SEC result of L51.

FIG. 33C shows melting curve of L51.

FIG. 33D shows VEGF binding results of L51, W366001-cAb1, and W366001-cAb2. NC is negative control. W366001-cAb1 (monovalent version) and W366001-cAb2 (bivalent version) are parental antibodies targeting VEGF.

FIG. 33E shows Ang-2 binding results of L51, W366001-cAb3, and W366001-cAb4. NC is negative control. W366001-cAb3 (monovalent version) and W366001-cAb4 (bivalent version) are parental antibodies targeting Ang-2.

FIG. 33F shows PD-1 binding results of L51, W366001-cAb11, and W366001-cAb12. NC is negative control. W366001-cAb11 (monovalent version) and W366001-cAb12 (bivalent version) are parental antibodies targeting PD-1.

FIG. 34A shows gel electrophoresis results of L57.

FIG. 34B shows SEC result of L57.

FIG. 34C shows melting curve of L57.

FIG. 34D shows VEGF binding results of L57, W366001-cAb1, and W366001-cAb2. NC is negative control. W366001-cAb1 (monovalent version) and W366001-cAb2 (bivalent version) are parental antibodies targeting VEGF.

FIG. 34E shows Ang-2 binding results of L57, W366001-cAb3, and W366001-cAb4. NC is negative control. W366001-cAb3 (monovalent version) and W366001-cAb4 (bivalent version) are parental antibodies targeting Ang-2.

FIG. 34F shows PD-1 binding results of L57, W366001-cAb11, and W366001-cAb12. NC is negative control. W366001-cAb11 (monovalent version) and W366001-cAb12 (bivalent version) are parental antibodies targeting PD-1.

FIG. 35A shows gel electrophoresis results of L58.

FIG. 35B shows SEC result of L58.

FIG. 35C shows melting curve of L58.

FIG. 35D shows VEGF binding results of L58, W366001-cAb1, and W366001-cAb2. NC is negative control. W366001-cAb1 (monovalent version) and W366001-cAb2 (bivalent version) are parental antibodies targeting VEGF.

FIG. 35E shows Ang-2 binding results of L58, W366001-cAb3, and W366001-cAb4. NC is negative control. W366001-cAb3 (monovalent version) and W366001-cAb4 (bivalent version) are parental antibodies targeting Ang-2.

FIG. 35F shows PD-1 binding results of L58, W366001-cAb11, and W366001-cAb12. NC is negative control. W366001-cAb11 (monovalent version) and W366001-cAb12 (bivalent version) are parental antibodies targeting PD-1.

FIG. 36A is a schematic structure of W366004-T1U1W1X1.N1-1.uIgG4V1 (or “N1”).

FIG. 36B is a schematic structure of W366004-T1U1W1X1.N2-1.uIgG4V1 (or “N2”).

FIG. 36C is a schematic structure of W366004-T1U1W1X1.N3-1.uIgG4V1 (or “N3”).

FIG. 36D is a schematic structure of W366004-T1U1W1X1.N4-1.uIgG4V1 (or “N4”).

FIG. 37A shows gel electrophoresis results of N1.

FIG. 37B shows SEC result of N1.

FIG. 37C shows melting curve of N1.

FIG. 37D shows VEGF binding results of N1, W366001-cAb1, and W366001-cAb2. NC is negative control. W366001-cAb1 (monovalent version) and W366001-cAb2 (bivalent version) are parental antibodies targeting VEGF.

FIG. 37E shows Ang-2 binding results of N1, W366001-cAb3, and W366001-cAb4. NC is negative control. W366001-cAb3 (monovalent version) and W366001-cAb4 (bivalent version) are parental antibodies targeting Ang-2.

FIG. 37F shows MSLN binding results of N1, W366001-cAb5, and W366001-cAb6. NC is negative control. W366001-cAb5 (monovalent version) and W366001-cAb6 (bivalent version) are parental antibodies targeting MSLN.

FIG. 37G shows GITR binding results of N1, W366001-cAb7, and W366001-cAb8. NC is negative control. W366001-cAb7 (monovalent version) and W366001-cAb8 (bivalent version) are parental antibodies targeting GITR.

FIG. 38A shows gel electrophoresis results of N2.

FIG. 38B shows SEC result of N2.

FIG. 38C shows melting curve of N2.

FIG. 38D shows VEGF binding results of N2, W366001-cAb1, and W366001-cAb2. NC is negative control. W366001-cAb1 (monovalent version) and W366001-cAb2 (bivalent version) are parental antibodies targeting VEGF.

FIG. 38E shows Ang-2 binding results of N2, W366001-cAb3, and W366001-cAb4. NC are parental antibodies targeting Ang-2.

FIG. 38F shows MSLN binding results of N2, W366001-cAb5, and W366001-cAb6. NC is negative control. W366001-cAb5 (monovalent version) and W366001-cAb6 (bivalent version) are parental antibodies targeting MSLN.

FIG. 38G shows GITR binding results of N2, W366001-cAb7, and W366001-cAb8. NC is negative control. W366001-cAb7 (monovalent version) and W366001-cAb8 (bivalent version) are parental antibodies targeting GITR.

FIG. 39A shows gel electrophoresis results of N3.

FIG. 39B shows SEC result of N3.

FIG. 39C shows melting curve of N3.

FIG. 39D shows VEGF binding results of N3, W366001-cAb1, and W366001-cAb2. NC is negative control. W366001-cAb1 (monovalent version) and W366001-cAb2 (bivalent version) are parental antibodies targeting VEGF.

FIG. 39E shows Ang-2 binding results of N3, W366001-cAb3, and W366001-cAb4. NC is negative control. W366001-cAb3 (monovalent version) and W366001-cAb4 (bivalent version) are parental antibodies targeting Ang-2.

FIG. 39F shows MSLN binding results of N3, W366001-cAb5, and W366001-cAb6. NC is negative control. W366001-cAb5 (monovalent version) and W366001-cAb6 (bivalent version) are parental antibodies targeting MSLN.

FIG. 39G shows PD-1 binding results of N3, W366001-cAb11, and W366001-cAb12. NC is negative control. W366001-cAb11 (monovalent version) and W366001-cAb12 (bivalent version) are parental antibodies targeting PD-1.

FIG. 40A shows gel electrophoresis results of N4.

FIG. 40B shows SEC result of N4.

FIG. 40C shows melting curve of N4.

FIG. 40D shows VEGF binding results of N4, W366001-cAb1, and W366001-cAb2. NC is negative control. W366001-cAb1 (monovalent version) and W366001-cAb2 (bivalent version) are parental antibodies targeting VEGF.

FIG. 40E shows Ang-2 binding results of N4, W366001-cAb3, and W366001-cAb4. NC is negative control. W366001-cAb3 (monovalent version) and W366001-cAb4 (bivalent version) are parental antibodies targeting Ang-2.

FIG. 40F shows MSLN binding results of N4, W366001-cAb5, and W366001-cAb6. NC is negative control. W366001-cAb5 (monovalent version) and W366001-cAb6 (bivalent version) are parental antibodies targeting MSLN.

FIG. 40G shows GITR binding results of N4, W366001-cAb7, and W366001-cAb8. NC is negative control. W366001-cAb7 (monovalent version) and W366001-cAb8 (bivalent version) are parental antibodies targeting GITR.

FIG. 41A is a schematic structure of W366003-T1U1W1.D38-1.His (or “D38”).

FIG. 41B shows gel electrophoresis results of D38.

FIG. 41C shows SEC result of D38.

FIG. 41D shows melting curve of D38.

FIG. 42 shows schematic structures of additional multispecific antigen binding protein formats.

FIG. 43A is a schematic structure of W366000-T1.V1-1.uIgG4V1 (or “V1”).

FIG. 43B is a schematic structure of W366000-T1.V2-1.uIgG4V1 (or “V2”).

FIG. 44A shows gel electrophoresis results of V1.

FIG. 44B shows SEC result of V1.

FIG. 44C shows melting curve of V1.

FIG. 44D shows VEGF binding results of V1, W366001-cAb1, and W366001-cAb2. NC is negative control. W366001-cAb1 (monovalent version) and W366001-cAb2 (bivalent version) are parental antibodies targeting VEGF.

FIG. 45A shows gel electrophoresis results of V2.

FIG. 45B shows SEC result of V2.

FIG. 45C shows melting curve of V2.

FIG. 45D shows VEGF binding results of V2, W366001-cAb1, and W366001-cAb2. NC is negative control. W366001-cAb1 (monovalent version) and W366001-cAb2 (bivalent version) are parental antibodies targeting VEGF.

FIG. 46A is a schematic structure of W366001-U1T1.H39-1.uIgG4V1 (or “H39”).

FIG. 46B is a schematic structure of W366001-U1T1.H40-1.uIgG4V1 (or “H40”).

FIG. 46C is a schematic structure of W366001-U1T1.V14-1.His (or “V14”).

FIG. 46D is a schematic structure of W366001-U1T1.V15-1.His (or “V15”).

FIG. 46E is a schematic structure of W366001-U1T1.V16-1.His (or “V16”).

FIG. 46F is a schematic structure of W366001-U1T1.V11-1.His (or “V11”).

FIG. 47A shows gel electrophoresis results of H39.

FIG. 47B shows SEC result of H39.

FIG. 47C shows melting curve of H39.

FIG. 47D shows VEGF binding results of H39, W366001-cAb1, and W366001-cAb2. NC is negative control. W366001-cAb1 (monovalent version) and W366001-cAb2 (bivalent version) are parental antibodies targeting VEGF.

FIG. 47E shows Ang-2 binding results of H39, W366001-cAb3, and W366001-cAb4. NC is negative control. W366001-cAb3 (monovalent version) and W366001-cAb4 (bivalent version) are parental antibodies targeting Ang-2.

FIG. 48A shows gel electrophoresis results of H40.

FIG. 48B shows SEC result of H40.

FIG. 48C shows melting curve of H40.

FIG. 48D shows VEGF binding results of H40, W366001-cAb1, and W366001-cAb2. NC is negative control. W366001-cAb1 (monovalent version) and W366001-cAb2 (bivalent version) are parental antibodies targeting VEGF.

FIG. 48E shows Ang-2 binding results of H40, W366001-cAb3, and W366001-cAb4. NC is negative control. W366001-cAb3 (monovalent version) and W366001-cAb4 (bivalent version) are parental antibodies targeting Ang-2.

FIG. 49A shows gel electrophoresis results of V14.

FIG. 49B shows SEC result of V14.

FIG. 49C shows melting curve of V14.

FIG. 49D shows VEGF binding results of V14. NC is negative control.

FIG. 49E shows Ang-2 binding results of V14. NC is negative control.

FIG. 50A shows gel electrophoresis results of V15.

FIG. 50B shows SEC result of V15.

FIG. 50C shows melting curve of V15.

FIG. 50D shows VEGF binding results of V15. NC is negative control.

FIG. 50E shows Ang-2 binding results of V15. NC is negative control.

FIG. 51A shows gel electrophoresis results of V16.

FIG. 51B shows SEC result of V16.

FIG. 51C shows melting curve of V16.

FIG. 51D shows VEGF binding results of V16. NC is negative control.

FIG. 51E shows Ang-2 binding results of V16. NC is negative control.

FIG. 52A shows gel electrophoresis results of V11.

FIG. 52B shows SEC result of V11.

FIG. 52C shows melting curve of V11.

FIG. 52D shows VEGF binding results of V11. NC is negative control.

FIG. 52E shows Ang-2 binding results of V11. NC is negative control.

FIG. 53A is a schematic structure of W366003-U1W3X1.D1-1 (or “D1”).

FIG. 53B is a schematic structure of W366003-U1W3X1.D2-1.His (or “D2”).

FIG. 53C is a schematic structure of W366003-U1W3X1.D3-1.His (or “D3”).

FIG. 53D is a schematic structure of W366003-U1W3X1.D43-1.His (or “D43”).

FIG. 53E is a schematic structure of W366003-U1W3X1.D44-1.His (or “D44”).

FIG. 54A shows gel electrophoresis results of D1.

FIG. 54B shows SEC result of D1.

FIG. 54C shows melting curve of D1.

FIG. 54D shows Ang-2 binding results of D1. NC is negative control.

FIG. 54E shows MSLN binding results of D1. NC is negative control.

FIG. 54F shows GITR binding results of D1. NC is negative control.

FIG. 55A shows gel electrophoresis results of D2.

FIG. 55B shows SEC result of D2.

FIG. 55C shows melting curve of D2.

FIG. 55D shows Ang-2 binding results of D2. NC is negative control.

FIG. 55E shows MSLN binding results of D2. NC is negative control.

FIG. 55F shows GITR binding results of D2. NC is negative control.

FIG. 56A shows gel electrophoresis results of D3.

FIG. 56B shows SEC result of D3.

FIG. 56C shows melting curve of D3.

FIG. 56D shows Ang-2 binding results of D3. NC is negative control.

FIG. 56E shows MSLN binding results of D3. NC is negative control.

FIG. 56F shows GITR binding results of D3. NC is negative control.

FIG. 57A shows gel electrophoresis results of D43.

FIG. 57B shows SEC result of D43.

FIG. 57C shows melting curve of D43.

FIG. 57D shows Ang-2 binding results of D43. NC is negative control.

FIG. 57E shows MSLN binding results of D43. NC is negative control.

FIG. 57F shows GITR binding results of D43. NC is negative control.

FIG. 58A shows gel electrophoresis results of D44.

FIG. 58B shows SEC result of D44.

FIG. 58C shows melting curve of D44.

FIG. 58D shows Ang-2 binding results of D44. NC is negative control.

FIG. 58E shows MSLN binding results of D44. NC is negative control.

FIG. 58F shows GITR binding results of D44. NC is negative control.

### DETAILED DESCRIPTION

While multispecific antibodies have a broad spectrum of use, developing and manufacturing multispecific antibodies remain to be a challenge. For example, the generation of bispecific IgG molecules is difficult due to the fact that the antigen-binding sites are built by the variable domains of the light and heavy chain (VL, VH). A bispecific IgG antibody requires two different heavy chains, and two different light chains, and exhibits asymmetry due to the presence of at least two different antigen binding sites. Promiscuous pairing of heavy and light chains of two antibodies expressed in one cell can theoretically result in 16 different combinations (10 different molecules), with only one being bispecific and the remaining pairings resulting in non-functional or monospecific molecules. To direct and to force correct assembly of correct binding sites, e.g., between heavy and light chains and two different heavy chains, is one of the challenges of generating multispecific antibodies.

The present disclosure provides a versatile multispecific antibody platform. Particularly, the antigen-binding site in the multispecific antigen binding proteins can fold properly and retain a high binding affinity with the antigen. In addition, the methods described herein can significantly reduce the chance of mispairing. Furthermore, the present disclosure demonstrate that these multispecific and multivalent antigen binding proteins can be easily expressed at a high level and can be readily purified and manufactured.

### Multispecific Antigen Binding Proteins

A multispecific antigen binding protein is an artificial protein that can simultaneously bind to two or more different types of epitopes. The epitopes can be in the same antigen or in different antigens. In some embodiments, a multispecific antigen binding protein can have two, three, four, five, six, or more antigen binding sites. In some embodiments, the antigen binding site comprises or consists of one heavy chain variable region (VH) and one light chain variable region (VL). In some embodiments, the antigen binding site comprises or consists of one VHH. In some embodiments, these antigen binding proteins comprise or consist of 1, 2, 3, 4, 5, 6, 7, 8, 9, 10, 11, 12 or more than 12 VHHs.

A VHH is an antibody variable domain that can specifically binds to an antigen. They are variable domains of a heavy chain antibody or single-domain antibodies. Heavy chain antibodies only have two heavy chains, which lack the CH1 region but still bear an antigen-binding domain at their N-terminus. Heavy chain antibodies are often obtained from camelids. Like all mammals, camelids (e.g., llamas) can produce conventional antibodies made of two heavy chains and two light chains bound together with disulfide bonds in a Y shape (e.g., IgG1). However, they also produce two unique subclasses of IgG: IgG2 and IgG3, also known as heavy chain antibody. Conventional Ig require the association of variable regions from both heavy and light chains to allow a high diversity of antigen-antibody interactions. Although isolated heavy and light chains still show this capacity, they exhibit very low affinity when compared to paired heavy and light chains. The unique feature of heavy chain antibody is the capacity of their monomeric antigen binding regions to bind antigens with specificity, affinity and especially diversity that are comparable to conventional antibodies without the need of pairing with another region. This feature is mainly due to a couple of major variations within the amino acid sequence of the variable region of the two heavy chains, which induce deep conformational changes when compared to conventional Ig.

The single variable domain of these antibodies (designated VHH, sdAb, nanobody, or heavy-chain antibody variable domain) is the smallest antigen-binding domain generated by adaptive immune systems. The third Complementarity Determining Region (CDR3) of the variable region of these antibodies has often been found to be twice as long as the conventional ones. This results in an increased interaction surface with the antigen as well as an increased diversity of antigen-antibody interactions, which compensates the absence of the light chains. With a long complementarity-determining region 3 (CDR3), VHHs can extend into crevices on proteins that are not accessible to conventional antibodies, including functionally interesting sites such as the active site of an enzyme or the receptor-binding canyon on a virus surface. Moreover, an additional cysteine residue allows the structure to be more stable, thus increasing the strength of the interaction.

VHHs offer numerous other advantages compared to conventional antibodies carrying variable domains (VH and VL) of conventional antibodies, including higher stability, solubility, expression yields, and refolding capacity, as well as better in vivo tissue penetration. Moreover, in contrast to the VH domains of conventional antibodies VHH do not display an intrinsic tendency to bind to light chains. This facilitates the induction of heavy chain antibodies in the presence of a functional light chain loci. Further, since VHH do not bind to VL domains, it is much easier to reformat VHHs into multispecific antibody constructs than constructs containing conventional VH-VL pairs or single domains based on VH domains.

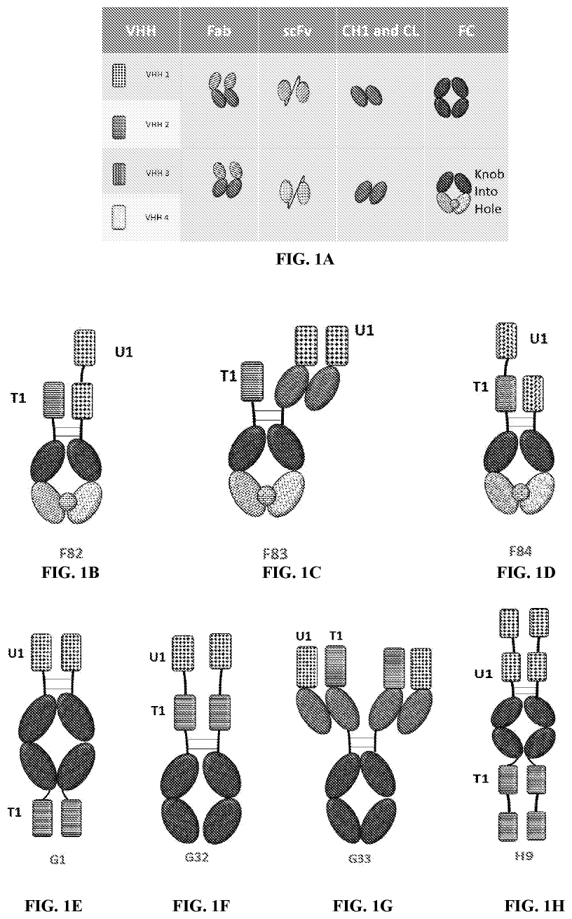

The present disclosure provides various formats for multispecific antigen binding proteins comprising one or more VHHs. Many of these formats are shown in FIGS. 1B-1H, FIGS. 9A-9F, FIGS. 16A-16H, FIGS. 25A-25J, FIGS. 36A-36D, and FIG. 41A. Schematic diagrams of structural elements are shown in FIG. 1A. Additional formats are shown in FIG. 42.

In some embodiments, the antigen binding proteins provided here comprise 1, 2, 3, 4, 5, 6, 7, 8, 9, 10, 11, 12 or more than 12 VHHs. In some embodiments, the antigen binding proteins provided here further comprise 1, 2, 3, 4, 5, 6, 7, 8, 9, 10, 11, 12 or more than 12 antigen binding sites, wherein these antigen binding sites are formed by a pair of VH and VL.

In some embodiments, these antigen binding sites are fused together. In some embodiments, these antigen binding sites are attached to a scaffold (e.g., a polypeptide, a full length antibody, an Fc). In some embodiments, the antigen binding proteins comprise an Fc. In some embodiments, one or more VHHs and/or one or more VH/VL pairs are linked to the Fc. In some embodiments, the one or more VHHs and/or one or more VH/VL pairs are linked to the Fc through a linker sequence, i.e., an amino acid sequence of at least one amino acid. In some embodiments, the linker sequence is a hinge region sequence, a CH1, or a CL. In some embodiments, the Fc is an IgG1, IgG2, IgG3, or IgG4 Fc. In some embodiments, the antigen binding protein comprises an Fc domain that can be originated from various types (e.g., IgG, IgE, IgM, IgD, IgA, and IgY), class (e.g., IgG1, IgG2, IgG3, IgG4, IgA1, and IgA2), or subclass. In some embodiments, the Fc domain is originated from an IgG antibody or antigen-binding fragment thereof. In some embodiments, the Fc domain comprises one, two, three, four, or more heavy chain constant regions.

In some embodiments, the antigen binding proteins comprise a full length antibody (e.g., IgG, IgG1, IgG2, IgG3, IgG4, IgA, IgD, IgE, or IgM). In some embodiments, the antigen binding protein comprises a full-length antibody consisting of two heavy chains and two light chains. In some embodiments, the full-length antibody is a full-length monoclonal antibody consisting of two identical heavy chains and two identical light chains. In some embodiments, one or more VHHs and/or one or more VH/VL pairs are linked to the C terminal of the heavy chain. In some embodiments, one or more VHHs and/or one or more VH/VL pairs are linked to the N terminal of the heavy chain. In some embodiments, one or more VHHs and/or one or more VH/VL pairs are linked to the C terminal of the light chain. In some embodiments, one or more VHHs and/or one or more VH/VL pairs are linked to the N terminal of the light chain.

In some embodiments, the antigen binding proteins can be derived from various antibody variants (including derivatives and conjugates) or antibody fragments and multi-specific (e.g., bi-specific) antibodies or antibody fragments. These antibodies provided herein include e.g., polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof.

Numerous formats for multispecific antigen binding proteins are provided. In one aspect, the disclosure provides a multispecific antigen binding protein. The antigen binding protein comprises or consists of 1, 2, 3, 4, 5, 6, or more than 6 Fc. The Fc can serve as a protein scaffold, wherein one or more VHHs can be linked, attached, or fused to the protein scaffold directly or indirectly. In some embodiments, the antigen binding protein comprises 1, 2, 3, 4, 5, 6, 7, 8, 9, 10, 11, 12 or more than 12 VHH. In some embodiments, the ratio of VHH to Fc in the antigen binding protein is at least or about 8:1, 7:1, 6:1, 5:1, 4:1, 3:1, 2:1, or 1:1.

In some embodiments, the antigen binding protein comprises or consists of 1, 2, 3, 4, 5, 6, 7, 8, 9, 10, 11, 12 or more than 12 VH-VL pairs, wherein the VH and VL associate with each other, forming an antigen binding site. In some embodiments, the ratio of VHH to VH-VL pairs in the antigen binding protein is at least or about 8:1, 7:1, 6:1, 5:1, 4:1, 3:1, 2:1, 1:1, 1:2, 1:3, or 1:4.

In some embodiments, the antigen binding protein does not have an Fc. The ratio of VHH to VH-VL pairs in the antigen binding protein is at least or about 8:1, 7:1, 6:1, 5:1, 4:1, 3:1, 2:1, 1:1, 1:2, 1:3, or 1:4.

In some embodiments, one or more VHHs are linked, fused, or attached to VH, CH1, CH2, CH3, CH4, e.g., directly or indirectly through a linker sequence. In some embodiments, one or more VHHs are linked, fused, or attached to VL or CL, e.g., directly or indirectly through a linker sequence. In some embodiments, one or more VHHs are linked to another VHH directly or indirectly through a linker sequence.

The VHHs can be linked to the N terminal or the C terminal of a polypeptide. In some embodiments, the VHHs are linked to the polypeptide through a linker sequence as described herein. In some embodiments, one or more VHHs are linked to the C terminal of CH3. In some embodiments, one or more VHHs are linked to the C terminal of CH1 or CL. In some embodiments, one or more VHHs are linked to the N terminal of CH2. In some embodiments, one or more VHHs are linked to the N terminal of VH or VL.

In some embodiments, the antigen binding protein comprises or consists of 1, 2, 3, 4, 5, 6, 7, 8 or more than 8 polypeptides. These polypeptides can have CH2 domains and/or CH3 domains. In some embodiments, they can have CH1 domains. In some embodiments, they can have CL domains. In some embodiments, they can have 1, 2, 3, 4, 5, 6, 7, 8, 9, 10, 11, 12 or more than 12 VHH. In some embodiments, they can have 1, 2, 3, 4, 5, 6, 7, 8, 9, 10, 11, 12 or more than 12 VHs or VLs.

In some embodiments, the antigen binding protein comprises or consists of a Fab-like domain. As used herein, a “Fab-like” domain refers to a structure that comprises a CH1 and a CL, wherein the CH1 and the CL associate with each other and form a dimer. In some embodiments, a VHH is linked to a CH1 and a VHH is linked to a CL.

In some embodiments, the antigen binding protein comprises or consists a first antigen binding site comprising or consisting of a VH and VL, and a second antigen binding site comprising or consisting of a VHH, wherein the first antigen binding site and the second antigen binding site are linked, fused, or attached to each other.

In some embodiments, the antigen binding protein comprises or consists of two polypeptides. The first polypeptide can comprise or consist of, e.g., preferably from N-terminus to C-terminus, a VHH, an optional hinge region sequence, a CH2 domain, and a CH3 domain. In some embodiments, the second polypeptide can comprise or consist of, e.g., preferably from N-terminus to C-terminus, a VHH, an optional hinge region sequence, a CH2 domain, and a CH3 domain. In some embodiments, the second polypeptide can comprise or consist of, e.g., preferably from N-terminus to C-terminus, two VHHs, an optional hinge region sequence, a CH2 domain, and a CH3 domain. In some embodiments, KIH mutations are introduced (e.g., in the CH3 domains). In some embodiments, the antigen binding protein comprises or consists of a structure as shown in FIG. 1B. In some embodiments, the VHH in the first polypeptide targets VEGF; and the two VHHs in the second polypeptide target Ang-2.

In some embodiments, the antigen binding protein comprises or consists of three polypeptides. The first polypeptide can comprise or consist of, e.g., preferably from N-terminus to C-terminus, a VHH, an optional hinge region sequence, a CH2 domain, and a CH3 domain. The second polypeptide can comprise or consist of, e.g., preferably from N-terminus to C-terminus, a VHH, a CH1 domain, an optional hinge region sequence, a CH2 domain, and a CH3 domain. The third polypeptide can comprise or consist of, e.g., preferably from N-terminus to C-terminus, a VHH and a CL domain. The CH1 domain and the CL domain can associate with each other. In some embodiments, KIH mutations are introduced (e.g., in the CH3 domains). In some embodiments, the antigen binding protein comprises or consists of a structure as shown in FIG. 1C. In some embodiments, the VHH in the first polypeptide targets VEGF; the VHHs in the second and the third polypeptides target Ang-2.

In some embodiments, the antigen binding protein comprises or consists of two polypeptides. In some embodiments, the first polypeptide can comprise or consist of, e.g., preferably from N-terminus to C-terminus, a first VHH, a second VHH, an optional hinge region sequence, a CH2 domain, and a CH3 domain. In some embodiments, the second polypeptide can comprise or consist of, e.g., preferably from N-terminus to C-terminus, a third VHH, an optional hinge region sequence, a CH2 domain, and a CH3 domain. In some embodiments, KIH mutations are introduced (e.g., in the CH3 domains). In some embodiments, the antigen binding protein comprises or consists of a structure as shown in FIG. 1D. In some embodiments, the first VHH and the third VHH target Ang-2; and the second VHH targets VEGF.

In some embodiments, the antigen binding protein comprises or consists of two polypeptides. The first polypeptide can comprise or consist of, e.g., preferably from N-terminus to C-terminus, a first VHH, an optional hinge region sequence, a CH2 domain, a CH3 domain, and a second VHH. In some embodiments, the second polypeptide can comprise or consist of, e.g., preferably from N-terminus to C-terminus, a first VHH, an optional hinge region sequence, a CH2 domain, a CH3 domain, and a second VHH. In some embodiments, the antigen binding protein comprises or consists of a structure as shown in FIG. 1E. In some embodiments, the first VHHs in the first polypeptide and in the second polypeptide target Ang-2; the second VHHs in the first polypeptide and in the second polypeptide target VEGF.

In some embodiments, the antigen binding protein comprises or consists of two polypeptides. The first polypeptide can comprise or consist of, e.g., preferably from N-terminus to C-terminus, a first VHH, a second VHH, an optional hinge region sequence, a CH2 domain, and a CH3 domain. In some embodiments, the second polypeptide can comprise or consist of, e.g., preferably from N-terminus to C-terminus, a first VHH, a second VHH, an optional hinge region sequence, a CH2 domain, and a CH3 domain. In some embodiments, the antigen binding protein comprises or consists of a structure as shown in FIG. 1F. In some embodiments, the first VHHs in the first polypeptide and in the second polypeptide target Ang-2; the second VHHs in the first polypeptide and in the second polypeptide target VEGF.

In some embodiments, the antigen binding protein comprises or consists of four polypeptides. The first polypeptide can comprise or consist of, e.g., preferably from N-terminus to C-terminus, a VHH, a CH1 domain, an optional hinge region sequence, a CH2 domain, and a CH3 domain. The second polypeptide can comprise or consist of, e.g., preferably from N-terminus to C-terminus, a VHH, a CH1 domain, an optional hinge region sequence, a CH2 domain, and a CH3 domain. The third polypeptide can comprise or consist of, e.g., preferably from N-terminus to C-terminus, a VHH and a CL domain. The fourth polypeptide can comprise or consist of, e.g., preferably from N-terminus to C-terminus, a VHH and a CL domain. The CH1 domain in the first polypeptide and the CL domain in the third polypeptide can associate with each other. The CH1 domain in the second polypeptide and the CL domain in the fourth polypeptide can associate with each other. In some embodiments, the antigen binding protein comprises or consists of a structure as shown in FIG. 1G. In some embodiments, the VHHs in the first polypeptide and in the second polypeptide target VEGF; the VHHs in the third polypeptide and the fourth polypeptides target Ang-2.

In some embodiments, the antigen binding protein comprises or consists of two polypeptides. The first polypeptide can comprise or consist of, e.g., preferably from N-terminus to C-terminus, two first VHHs, an optional hinge region sequence, a CH2 domain, a CH3 domain, and two second VHHs. In some embodiments, the second polypeptide can comprise or consist of, e.g., preferably from N-terminus to C-terminus, two first VHHs, an optional hinge region sequence, a CH2 domain, a CH3 domain, and two second VHHs. In some embodiments, the antigen binding protein comprises or consists of a structure as shown in FIG. 1H. In some embodiments, the two first VHHs in the first polypeptide and in the second polypeptide target Ang-2; the two second VHHs in the first polypeptide and in the second polypeptide target VEGF.

In some embodiments, the antigen binding protein comprises or consists of two polypeptides. The first polypeptide can comprise or consist of, e.g., preferably from N-terminus to C-terminus, a VHH, an optional hinge region sequence, a CH2 domain, and a CH3 domain. In some embodiments, the second polypeptide can comprise or consist of, e.g., preferably from N-terminus to C-terminus, a VHH, an optional hinge region sequence, a CH2 domain, and a CH3 domain. In some embodiments, KIH mutations are introduced (e.g., in the CH3 domains). In some embodiments, the antigen binding protein comprises or consists of a structure as shown in FIG. 9A. In some embodiments, the VHH in the first polypeptide targets VEGF, and the VHH in the second polypeptide targets Ang-2.

In some embodiments, the antigen binding protein comprises or consists of three polypeptides. The first polypeptide can comprise or consist of, e.g., preferably from N-terminus to C-terminus, a first VHH, a second VHH, an optional hinge region sequence, a CH2 domain, and a CH3 domain. The second polypeptide can comprise or consist of, e.g., preferably from N-terminus to C-terminus, a VHH, a CH1 domain, an optional hinge region sequence, a CH2 domain, and a CH3 domain. The third polypeptide can comprise or consist of, e.g., preferably from N-terminus to C-terminus, a VHH and a CL domain. The CH1 domain and the CL domain can associate with each other. In some embodiments, KIH mutations are introduced (e.g., in the CH3 domains). In some embodiments, the antigen binding protein comprises or consists of a structure as shown in FIG. 9B. In some embodiments, the second VHH in the first polypeptide targets VEGF; the first VHH in the first polypeptide, the VHHs in the second polypeptide and in the third polypeptides target Ang-2.

In some embodiments, the antigen binding protein comprises or consists of four polypeptides. The first polypeptide can comprise or consist of, e.g., preferably from N-terminus to C-terminus, a VHH, a CH1 domain, an optional hinge region sequence, a CH2 domain, and a CH3 domain. The second polypeptide can comprise or consist of, e.g., preferably from N-terminus to C-terminus, a VHH, a CH1 domain, an optional hinge region sequence, a CH2 domain, and a CH3 domain. The third polypeptide can comprise or consist of, e.g., preferably from N-terminus to C-terminus, a VHH and a CL domain. The fourth polypeptide can comprise or consist of, e.g., preferably from N-terminus to C-terminus, a VHH and a CL domain. The CH1 domain in the first polypeptide and the CL domain in the third polypeptide can associate with each other. The CH1 domain in the second polypeptide and the CL domain in the fourth polypeptide can associate with each other. In some embodiments, KIH mutations are introduced (e.g., in the CH3 domains). In some embodiments, the antigen binding protein comprises or consists of a structure as shown in FIG. 9C. In some embodiments, the VHH in the first polypeptide targets VEGF; the VHHs in the second polypeptide, the third polypeptide and the fourth polypeptides target Ang-2.

In some embodiments, the antigen binding protein comprises or consists of two polypeptides. The first polypeptide can comprise or consist of, e.g., preferably from N-terminus to C-terminus, a first VHH, a second VHH, an optional hinge region sequence, a CH2 domain, and a CH3 domain. In some embodiments, the second polypeptide can comprise or consist of, e.g., preferably from N-terminus to C-terminus, a first VHH, a second VHH, an optional hinge region sequence, a CH2 domain, and a CH3 domain. In some embodiments, KIH mutations are introduced (e.g., in the CH3 domains). In some embodiments, the antigen binding protein comprises or consists of a structure as shown in FIG. 9D. In some embodiments, the first VHH in the first polypeptide, the first VHH and the second VHH in the second polypeptide target Ang-2; and the second VHH in the first polypeptide targets VEGF.

In some embodiments, the antigen binding protein comprises or consists of four polypeptides. The first polypeptide can comprise or consist of, e.g., preferably from N-terminus to C-terminus, a first VHH, a CH1 domain, an optional hinge region sequence, a CH2 domain, a CH3 domain and a second VHH. The second polypeptide can comprise or consist of, e.g., preferably from N-terminus to C-terminus, a first VHH, a CH1 domain, an optional hinge region sequence, a CH2 domain, a CH3 domain, and a second VHH. The third polypeptide can comprise or consist of, e.g., preferably from N-terminus to C-terminus, a VHH and a CL domain. The fourth polypeptide can comprise or consist of, e.g., preferably from N-terminus to C-terminus, a VHH and a CL domain. The CH1 domain in the first polypeptide and the CL domain in the third polypeptide can associate with each other. The CH1 domain in the second polypeptide and the CL domain in the fourth polypeptide can associate with each other. In some embodiments, the antigen binding protein comprises or consists of a structure as shown in FIG. 9E. In some embodiments, the first VHHs in the first polypeptide and in the second polypeptide, the VHHs in the third polypeptide and in the fourth polypeptide target Ang-2; the second VHHs in the first polypeptide and the second polypeptides target VEGF.

In some embodiments, the antigen binding protein comprises or consists of two polypeptides. The first polypeptide can comprise or consist of, e.g., preferably from N-terminus to C-terminus, two first VHHs, an optional hinge region sequence, a CH2 domain, a CH3 domain, and a second VHH. In some embodiments, the second polypeptide can comprise or consist of, e.g., preferably from N-terminus to C-terminus, two first VHHs, an optional hinge region sequence, a CH2 domain, a CH3 domain, and a second VHH. In some embodiments, the antigen binding protein comprises or consists of a structure as shown in FIG. 9F. In some embodiments, the two first VHHs in the first polypeptide and in the second polypeptide target Ang-2; the second VHHs in the first polypeptide and in the second polypeptide target VEGF.

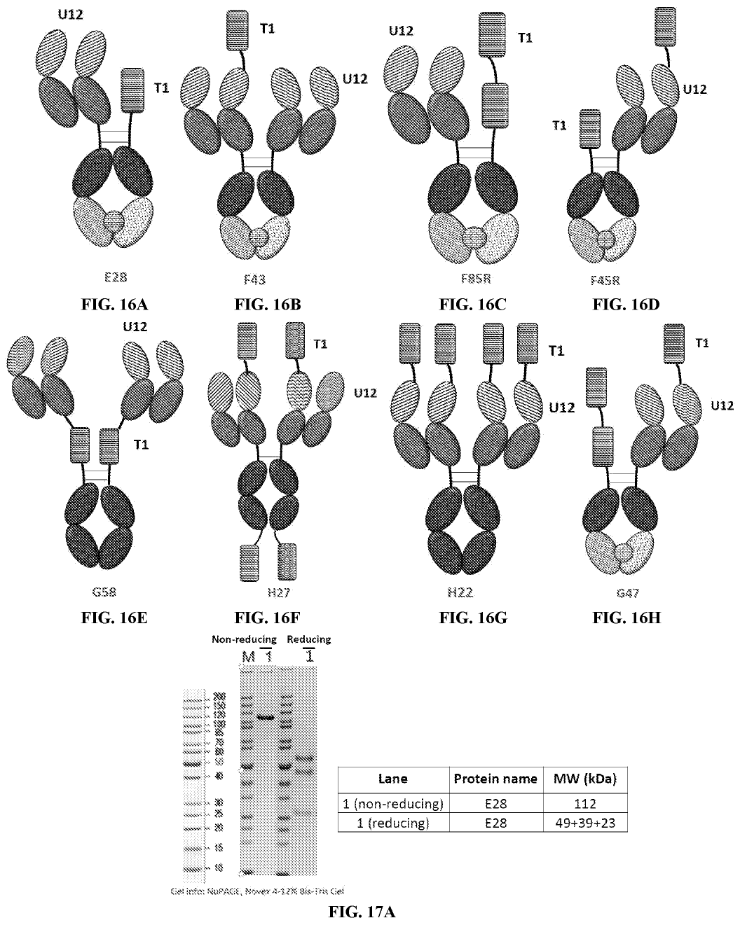

In some embodiments, the antigen binding protein comprises or consists of three polypeptides. The first polypeptide can comprise or consist of, e.g., preferably from N-terminus to C-terminus, a VH, a CH1 domain, an optional hinge region sequence, a CH2 domain, and a CH3 domain. The second polypeptide can comprise or consist of, e.g., preferably from N-terminus to C-terminus, a VHH, an optional hinge region sequence, a CH2 domain, and a CH3 domain. The third polypeptide can comprise or consist of, e.g., preferably from N-terminus to C-terminus, a VL and a CL domain. The CH1 domain and the CL domain can associate with each other. In some embodiments, KIH mutations are introduced (e.g., in the CH3 domains). In some embodiments, the antigen binding protein comprises or consists of a structure as shown in FIG. 16A. In some embodiments, the VHH in the second polypeptide targets VEGF; the VH in the first polypeptide and the VL in the third polypeptide associate with each other, forming an antigen-binding site that targets PD-1.

In some embodiments, the antigen binding protein comprises or consists of four polypeptides. The first polypeptide can comprise or consist of, e.g., preferably from N-terminus to C-terminus, a VHH, a first VH, a CH1 domain, an optional hinge region sequence, a CH2 domain, and a CH3 domain. The second polypeptide can comprise or consist of, e.g., preferably from N-terminus to C-terminus, a second VH, a CH1 domain, an optional hinge region sequence, a CH2 domain, and a CH3 domain. The third polypeptide can comprise or consist of, e.g., preferably from N-terminus to C-terminus, a first VL and a CL domain. The fourth polypeptide can comprise or consist of, e.g., preferably from N-terminus to C-terminus, a second VL and a CL domain. The CH1 domain in the first polypeptide and the CL domain in the third polypeptide can associate with each other. The CH1 domain in the second polypeptide and the CL domain in the fourth polypeptide can associate with each other. In some embodiments, KIH mutations are introduced (e.g., in the CH3 domains). In some embodiments, the antigen binding protein comprises or consists of a structure as shown in FIG. 16B. In some embodiments, the VHH in the first polypeptide targets VEGF; the first VH and the first VL associate with each other, forming a first antigen-binding site that targets PD-1; the second VH and the second VL associate with each other, forming a second antigen-binding site that targets PD-1.

In some embodiments, the antigen binding protein comprises or consists of three polypeptides. The first polypeptide can comprise or consist of, e.g., preferably from N-terminus to C-terminus, a VH, a CH1 domain, an optional hinge region sequence, a CH2 domain, and a CH3 domain. The second polypeptide can comprise or consist of, e.g., preferably from N-terminus to C-terminus, a VL and a CL domain. The third polypeptide can comprise or consist of, e.g., preferably from N-terminus to C-terminus, two VHHs, an optional hinge region sequence, a CH2 domain, and a CH3 domain. The CH1 domain in the first polypeptide and the CL domain in the second polypeptide can associate with each other. In some embodiments, KIH mutations are introduced (e.g., in the CH3 domains). In some embodiments, the antigen binding protein comprises or consists of a structure as shown in FIG. 16C. In some embodiments, the two VHHs in the third polypeptide target VEGF; the VH in the first polypeptide and the VL in the second polypeptide associate with each other, forming an antigen-binding site that targets PD-1.

In some embodiments, the antigen binding protein comprises or consists of three polypeptides. The first polypeptide can comprise or consist of, e.g., preferably from N-terminus to C-terminus, a VHH, an optional hinge region sequence, a CH2 domain, and a CH3 domain. The second polypeptide can comprise or consist of, e.g., preferably from N-terminus to C-terminus, a VH, a CH1 domain, an optional hinge region sequence, a CH2 domain, and a CH3 domain. The third polypeptide can comprise or consist of, e.g., preferably from N-terminus to C-terminus, a VHH, a VL, and a CL domain. The CH1 domain in the second polypeptide and the CL domain in the third polypeptide can associate with each other. In some embodiments, KIH mutations are introduced (e.g., in the CH3 domains). In some embodiments, the antigen binding protein comprises or consists of a structure as shown in FIG. 16D. In some embodiments, the VHHs in the first and third polypeptide target VEGF; the VH in the second polypeptide and the VL in the third polypeptide associate with each other, forming an antigen-binding site that targets PD-1.

In some embodiments, the antigen binding protein comprises or consists of four polypeptides. The first polypeptide can comprise or consist of, e.g., preferably from N-terminus to C-terminus, a first VH, a first CH1 domain, a VHH, an optional hinge region sequence, a CH2 domain, and a CH3 domain. The second polypeptide can comprise or consist of, e.g., preferably from N-terminus to C-terminus, a first VL and a first CL domain. The third polypeptide can comprise or consist of, e.g., preferably from N-terminus to C-terminus, a second VH, a second CH1 domain, a VHH, an optional hinge region sequence, a CH2 domain, and a CH3 domain. The fourth polypeptide can comprise or consist of, e.g., preferably from N-terminus to C-terminus, a second VL and a second CL domain. The first CH1 domain and the first CL domain can associate with each other. The second CH1 domain and the second CL domain can associate with each other. In some embodiments, the antigen binding protein comprises or consists of a structure as shown in FIG. 16E. In some embodiments, the VHHs in the first and third polypeptide target VEGF; the first VH and the first VL associate with each other, forming a first antigen-binding site that targets PD-1; the second VH and the second VL associate with each other, forming a second antigen-binding site that targets PD-1.

In some embodiments, the antigen binding protein comprises or consists of four polypeptides. The first polypeptide can comprise or consist of, e.g., preferably from N-terminus to C-terminus, a first VHH, a first VH, a first CH1 domain, an optional hinge region sequence, a CH2 domain, a CH3 domain, and a second VHH. The second polypeptide can comprise or consist of, e.g., preferably from N-terminus to C-terminus, a first VL and a first CL domain. The third polypeptide can comprise or consist of, e.g., preferably from N-terminus to C-terminus, a third VHH, a second VH, a second CH1 domain, an optional hinge region sequence, a CH2 domain, a CH3 domain, and a fourth VHH. The fourth polypeptide can comprise or consist of, e.g., preferably from N-terminus to C-terminus, a second VL and a second CL domain. The first CH1 domain and the first CL domain can associate with each other. The second CH1 domain and the second CL domain can associate with each other. In some embodiments, the antigen binding protein comprises or consists of a structure as shown in FIG. 16F. In some embodiments, the first VHH, the second VHH, the third VHH, and the fourth VHH target VEGF; the first VH and the first VL associate with each other, forming a first antigen-binding site that targets PD-1; the second VH and the second VL associate with each other, forming a second antigen-binding site that targets PD-1.

In some embodiments, the antigen binding protein comprises or consists of four polypeptides. The first polypeptide can comprise or consist of, e.g., preferably from N-terminus to C-terminus, a VHH, a first VH, a first CH1 domain, an optional hinge region sequence, a CH2 domain, and a CH3 domain. The second polypeptide can comprise or consist of, e.g., preferably from N-terminus to C-terminus, a VHH, a first VL and a first CL domain. The third polypeptide can comprise or consist of, e.g., preferably from N-terminus to C-terminus, a VHH, a second VH, a second CH1 domain, an optional hinge region sequence, a CH2 domain, and a CH3 domain. The fourth polypeptide can comprise or consist of, e.g., preferably from N-terminus to C-terminus, a VHH, a second VL and a second CL domain. The first CH1 domain and the first CL domain can associate with each other. The second CH1 domain and the second CL domain can associate with each other. In some embodiments, the antigen binding protein comprises or consists of a structure as shown in FIG. 16G. In some embodiments, the VHHs in the first, second, third, and fourth polypeptides target VEGF; the first VH and the first VL associate with each other, forming a first antigen-binding site that targets PD-1; the second VH and the second VL associate with each other, forming a second antigen-binding site that targets PD-1.

In some embodiments, the antigen binding protein comprises or consists of three polypeptides. The first polypeptide can comprise or consist of, e.g., preferably from N-terminus to C-terminus, two VHHs, an optional hinge region sequence, a CH2 domain, and a CH3 domain. The second polypeptide can comprise or consist of, e.g., preferably from N-terminus to C-terminus, a VH, a CH1 domain, an optional hinge region sequence, a CH2 domain, and a CH3 domain. The third polypeptide can comprise or consist of, e.g., preferably from N-terminus to C-terminus, a VHH, a VL and a CL domain. The CH1 domain in the second polypeptide and the CL domain in the third polypeptide can associate with each other. In some embodiments, KIH mutations are introduced (e.g., in the CH3 domains). In some embodiments, the antigen binding protein comprises or consists of a structure as shown in FIG. 16H. In some embodiments, the VHHs in the first and third polypeptides target VEGF; the VH and the VL associate with each other, forming an antigen-binding site that targets PD-1.

In some embodiments, the antigen binding protein comprises or consists of three polypeptides. The first polypeptide can comprise or consist of, e.g., preferably from N-terminus to C-terminus, a VH, a CH1 domain, an optional hinge region sequence, a CH2 domain, and a CH3 domain. The second polypeptide can comprise or consist of, e.g., preferably from N-terminus to C-terminus, a VL and a CL domain. The third polypeptide can comprise or consist of, e.g., preferably from N-terminus to C-terminus, a first VHH, a second VHH, an optional hinge region sequence, a CH2 domain, and a CH3 domain. The CH1 domain in the first polypeptide and the CL domain in the second polypeptide can associate with each other. In some embodiments, KIH mutations are introduced (e.g., in the CH3 domains). In some embodiments, the antigen binding protein comprises or consists of a structure as shown in FIG. 25A. In some embodiments, the first VHH in the third polypeptides targets VEGF; the first VHH in the third polypeptides targets Ang-2; the VH and the VL associate with each other, forming an antigen-binding site that targets PD-1.

In some embodiments, the antigen binding protein comprises or consists of four polypeptides. The first polypeptide can comprise or consist of, e.g., preferably from N-terminus to C-terminus, a first VH, a CH1 domain, an optional hinge region sequence, a CH2 domain, a CH3 domain, and a first VHH. The second polypeptide can comprise or consist of, e.g., preferably from N-terminus to C-terminus, a second VH, a CH1 domain, an optional hinge region sequence, a CH2 domain, a CH3 domain, and a second VHH. The third polypeptide can comprise or consist of, e.g., preferably from N-terminus to C-terminus, a first VL and a CL domain. The fourth polypeptide can comprise or consist of, e.g., preferably from N-terminus to C-terminus, a second VL and a CL domain. The CH1 domain in the first polypeptide and the CL domain in the third polypeptide can associate with each other. The CH1 domain in the second polypeptide and the CL domain in the fourth polypeptide can associate with each other. In some embodiments, KIH mutations are introduced (e.g., in the CH3 domains). In some embodiments, the antigen binding protein comprises or consists of a structure as shown in FIG. 25B. In some embodiments, the first VHH in the first polypeptide targets VEGF; the second VHH in the second polypeptide targets Ang-2; the first VH and the first VL associate with each other, forming a first antigen-binding site that targets PD-1; the second VH and the second VL associate with each other, forming a second antigen-binding site that targets PD-1.

In some embodiments, the antigen binding protein comprises or consists of two polypeptides. The first polypeptide can comprise or consist of, e.g., preferably from N-terminus to C-terminus, a first VHH, a second VHH, an optional hinge region sequence, a CH2 domain, a CH3 domain, and a third VHH. In some embodiments, the second polypeptide can comprise or consist of, e.g., preferably from N-terminus to C-terminus, a first VHH, a second VHH, an optional hinge region sequence, a CH2 domain, a CH3 domain, and a third VHH. In some embodiments, the antigen binding protein comprises or consists of a structure as shown in FIG. 25C. In some embodiments, the first VHHs in the first and second polypeptide target VEGF; the second VHHs in the first and second polypeptide target Ang-2; the third VHHs in the first and second polypeptide target MSLN.

In some embodiments, the antigen binding protein comprises or consists of four polypeptides. The first polypeptide can comprise or consist of, e.g., preferably from N-terminus to C-terminus, a first VHH, a first VH, a first CH1 domain, an optional hinge region sequence, a CH2 domain, a CH3 domain, and a second VHH. The second polypeptide can comprise or consist of, e.g., preferably from N-terminus to C-terminus, a first VL and a first CL domain. The third polypeptide can comprise or consist of, e.g., preferably from N-terminus to C-terminus, a third VHH, a second VH, a second CH1 domain, an optional hinge region sequence, a CH2 domain, a CH3 domain, and a fourth VHH. The fourth polypeptide can comprise or consist of, e.g., preferably from N-terminus to C-terminus, a second VL and a second CL domain. The first CH1 domain and the first CL domain can associate with each other. The second CH1 domain and the second CL domain can associate with each other. In some embodiments, the antigen binding protein comprises or consists of a structure as shown in FIG. 25D. In some embodiments, the first VHH and the third VHH target VEGF; the second VHH and the fourth VHH target Ang-2; the first VH and the first VL associate with each other, forming a first antigen-binding site that targets PD-1; the second VH and the second VL associate with each other, forming a second antigen-binding site that targets PD-1.

In some embodiments, the antigen binding protein comprises or consists of two polypeptides. The first polypeptide can comprise or consist of, e.g., preferably from N-terminus to C-terminus, a first VHH, a second VHH, an optional hinge region sequence, a CH2 domain, and a CH3 domain. In some embodiments, the second polypeptide can comprise or consist of, e.g., preferably from N-terminus to C-terminus, a first VHH, a second VHH, an optional hinge region sequence, a CH2 domain, and a CH3 domain. In some embodiments, KIH mutations are introduced (e.g., in the CH3 domains). In some embodiments, the antigen binding protein comprises or consists of a structure as shown in FIG. 25E. In some embodiments, the first VHH in the first polypeptide and the first VHH in the second polypeptide target VEGF; the second VHH in the first polypeptide targets Ang-2; and the second VHH in the second polypeptide targets MSLN.

In some embodiments, the antigen binding protein comprises or consists of four polypeptides. The first polypeptide can comprise or consist of, e.g., preferably from N-terminus to C-terminus, a VHH, a CH1 domain, an optional hinge region sequence, a CH2 domain, and a CH3 domain. The second polypeptide can comprise or consist of, e.g., preferably from N-terminus to C-terminus, a VHH, a CH1 domain, an optional hinge region sequence, a CH2 domain, a CH3 domain. The third polypeptide can comprise or consist of, e.g., preferably from N-terminus to C-terminus, a VHH and a CL domain. The fourth polypeptide can comprise or consist of, e.g., preferably from N-terminus to C-terminus, a VHH and a CL domain. The CH1 domain in the first polypeptide and the CL domain in the third polypeptide can associate with each other. The CH1 domain in the second polypeptide and the CL domain in the fourth polypeptide can associate with each other. In some embodiments, KIH mutations are introduced (e.g., in the CH3 domains). In some embodiments, the antigen binding protein comprises or consists of a structure as shown in FIG. 25F. In some embodiments, the VHH in the first polypeptide targets Ang-2; the VHH in the second polypeptide targets MSLN; the VHHs in the third and fourth polypeptides target VEGF.

In some embodiments, the antigen binding protein comprises or consists of three polypeptides. The first polypeptide can comprise or consist of, e.g., preferably from N-terminus to C-terminus, a first VHH, a second VHH, an optional hinge region sequence, a CH2 domain, and a CH3 domain. The second polypeptide can comprise or consist of, e.g., preferably from N-terminus to C-terminus, a VHH, a CH1 domain, an optional hinge region sequence, a CH2 domain, and a CH3 domain. The third polypeptide can comprise or consist of, e.g., preferably from N-terminus to C-terminus, a VHH and a CL domain. The CH1 domain and the CL domain can associate with each other. In some embodiments, KIH mutations are introduced (e.g., in the CH3 domains). In some embodiments, the antigen binding protein comprises or consists of a structure as shown in FIG. 25G. In some embodiments, the first VHH in the first polypeptide and the VHH in the third polypeptide target VEGF; the second VHH in the first polypeptide targets Ang-2; and the VHH in the second polypeptide targets MSLN.

In some embodiments, the antigen binding protein comprises or consists of four polypeptides. The first polypeptide can comprise or consist of, e.g., preferably from N-terminus to C-terminus, a VHH, a first VH, a first CH1 domain, an optional hinge region sequence, a CH2 domain, and a CH3 domain. The second polypeptide can comprise or consist of, e.g., preferably from N-terminus to C-terminus, a first VL and a first CL domain. The third polypeptide can comprise or consist of, e.g., preferably from N-terminus to C-terminus, a VHH, a second VH, a second CH1 domain, an optional hinge region sequence, a CH2 domain, and a CH3 domain. The fourth polypeptide can comprise or consist of, e.g., preferably from N-terminus to C-terminus, a second VL and a second CL domain. The first CH1 domain and the first CL domain can associate with each other. The second CH1 domain and the second CL domain can associate with each other. In some embodiments, KIH mutations are introduced (e.g., in the CH3 domains). In some embodiments, the antigen binding protein comprises or consists of a structure as shown in FIG. 25H. In some embodiments, the VHH in the first polypeptide targets VEGF; the VHH in the third polypeptide targets Ang-2; the first VH and the first VL associate with each other, forming a first antigen-binding site that targets PD-1; the second VH and the second VL associate with each other, forming a second antigen-binding site that targets PD-1.

In some embodiments, the antigen binding protein comprises or consists of three polypeptides. The first polypeptide can comprise or consist of, e.g., preferably from N-terminus to C-terminus, a first VHH, a second VHH, an optional hinge region sequence, a CH2 domain, and a CH3 domain. The second polypeptide can comprise or consist of, e.g., preferably from N-terminus to C-terminus, a VHH, a VH, a CH1 domain, an optional hinge region sequence, a CH2 domain, and a CH3 domain. The third polypeptide can comprise or consist of, e.g., preferably from N-terminus to C-terminus, a VL and a CL domain. The CH1 domain in the second polypeptide and the CL domain in the third polypeptide can associate with each other. In some embodiments, KIH mutations are introduced (e.g., in the CH3 domains). In some embodiments, the antigen binding protein comprises or consists of a structure as shown in FIG. 25I. In some embodiments, the first VHH in the first polypeptide and the VHH in the second polypeptide target VEGF; the second VHH in the first polypeptide targets Ang-2; the VH and the VL associate with each other, forming an antigen-binding site that targets PD-1.

In some embodiments, the antigen binding protein comprises or consists of four polypeptides. The first polypeptide can comprise or consist of, e.g., preferably from N-terminus to C-terminus, a first VH, a first CH1 domain, a VHH, an optional hinge region sequence, a CH2 domain, and a CH3 domain. The second polypeptide can comprise or consist of, e.g., preferably from N-terminus to C-terminus, a first VL and a first CL domain. The third polypeptide can comprise or consist of, e.g., preferably from N-terminus to C-terminus, a second VH, a second CH1 domain, a VHH, an optional hinge region sequence, a CH2 domain, and a CH3 domain. The fourth polypeptide can comprise or consist of, e.g., preferably from N-terminus to C-terminus, a second VL and a second CL domain. The first CH1 domain and the first CL domain can associate with each other. The second CH1 domain and the second CL domain can associate with each other. In some embodiments, KIH mutations are introduced (e.g., in the CH3 domains). In some embodiments, the antigen binding protein comprises or consists of a structure as shown in FIG. 25J. In some embodiments, the VHH in the first polypeptide targets VEGF; the VHH in the third polypeptide targets Ang-2; the first VH and the first VL associate with each other, forming a first antigen-binding site that targets PD-1; the second VH and the second VL associate with each other, forming a second antigen-binding site that targets PD-1.

In some embodiments, the antigen binding protein comprises or consists of three polypeptides. The first polypeptide can comprise or consist of, e.g., preferably from N-terminus to C-terminus, a first VHH, a second VHH, an optional hinge region sequence, a CH2 domain, and a CH3 domain. The second polypeptide can comprise or consist of, e.g., preferably from N-terminus to C-terminus, a VHH, a CH1 domain, an optional hinge region sequence, a CH2 domain, and a CH3 domain. The third polypeptide can comprise or consist of, e.g., preferably from N-terminus to C-terminus, a VHH and a CL domain. The CH1 domain and the CL domain can associate with each other. In some embodiments, KIH mutations are introduced (e.g., in the CH3 domains). In some embodiments, the antigen binding protein comprises or consists of a structure as shown in FIG. 36A. In some embodiments, the first VHH in the first polypeptide targets VEGF; the second VHH in the first polypeptide targets Ang-2; the VHH in the second polypeptide targets MSLN; and the VHH in the third polypeptide targets GITR.

In some embodiments, the antigen binding protein comprises or consists of two polypeptides. The first polypeptide can comprise or consist of, e.g., preferably from N-terminus to C-terminus, a first VHH, a second VHH, an optional hinge region sequence, a CH2 domain, and a CH3 domain. In some embodiments, the second polypeptide can comprise or consist of, e.g., preferably from N-terminus to C-terminus, a first VHH, a second VHH, an optional hinge region sequence, a CH2 domain, and a CH3 domain. In some embodiments, KIH mutations are introduced (e.g., in the CH3 domains). In some embodiments, the antigen binding protein comprises or consists of a structure as shown in FIG. 36B. In some embodiments, the first VHH in the first polypeptide targets VEGF; the second VHH in the first polypeptide targets Ang-2; the first VHH in the second polypeptide targets MSLN; and the second VHH in the second polypeptide targets GITR.

In some embodiments, the antigen binding protein comprises or consists of three polypeptides. The first polypeptide can comprise or consist of, e.g., preferably from N-terminus to C-terminus, a first VHH, a second VHH, an optional hinge region sequence, a CH2 domain, and a CH3 domain. The second polypeptide can comprise or consist of, e.g., preferably from N-terminus to C-terminus, a VHH, a VH, a CH1 domain, an optional hinge region sequence, a CH2 domain, and a CH3 domain. The third polypeptide can comprise or consist of, e.g., preferably from N-terminus to C-terminus, a VL and a CL domain. The CH1 domain in the second polypeptide and the CL domain in the third polypeptide can associate with each other. In some embodiments, KIH mutations are introduced (e.g., in the CH3 domains). In some embodiments, the antigen binding protein comprises or consists of a structure as shown in FIG. 36C. In some embodiments, the first VHH in the first polypeptide targets VEGF; the second VHH in the first polypeptide targets Ang-2; the VHH in the second polypeptide targets MSLN; the VH and the VL associate with each other, forming an antigen-binding site that targets PD-1.

In some embodiments, the antigen binding protein comprises or consists of two polypeptides. The first polypeptide can comprise or consist of, e.g., preferably from N-terminus to C-terminus, a first VHH, a second VHH, an optional hinge region sequence, a CH2 domain, a CH3 domain, a third VHH, and a fourth VHH. In some embodiments, the second polypeptide can comprise or consist of, e.g., preferably from N-terminus to C-terminus, a first VHH, a second VHH, an optional hinge region sequence, a CH2 domain, a CH3 domain, a third VHH, and a fourth VHH. In some embodiments, the antigen binding protein comprises or consists of a structure as shown in FIG. 36D. In some embodiments, the first VHHs in the first and second polypeptides target VEGF; the second VHHs in the first and second polypeptides target Ang-2; the third VHHs in the first and second polypeptides target MSLN; the fourth VHHs in the first and second polypeptides target GITR.

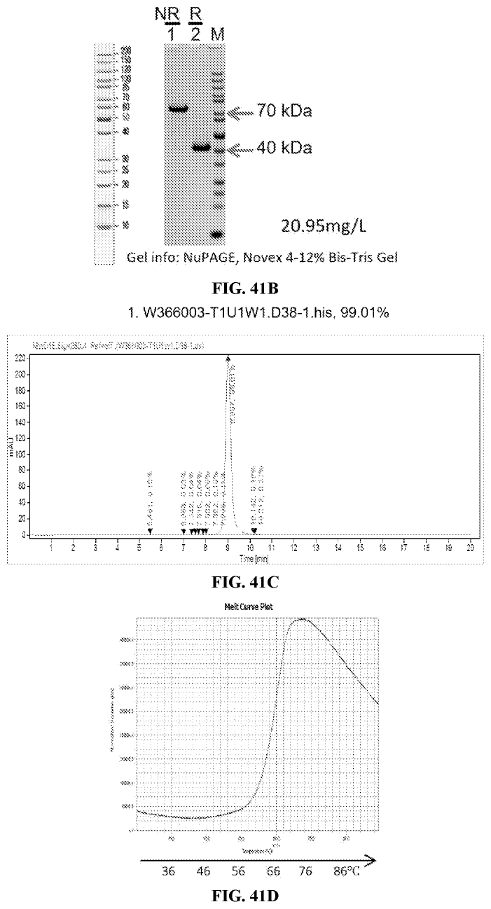

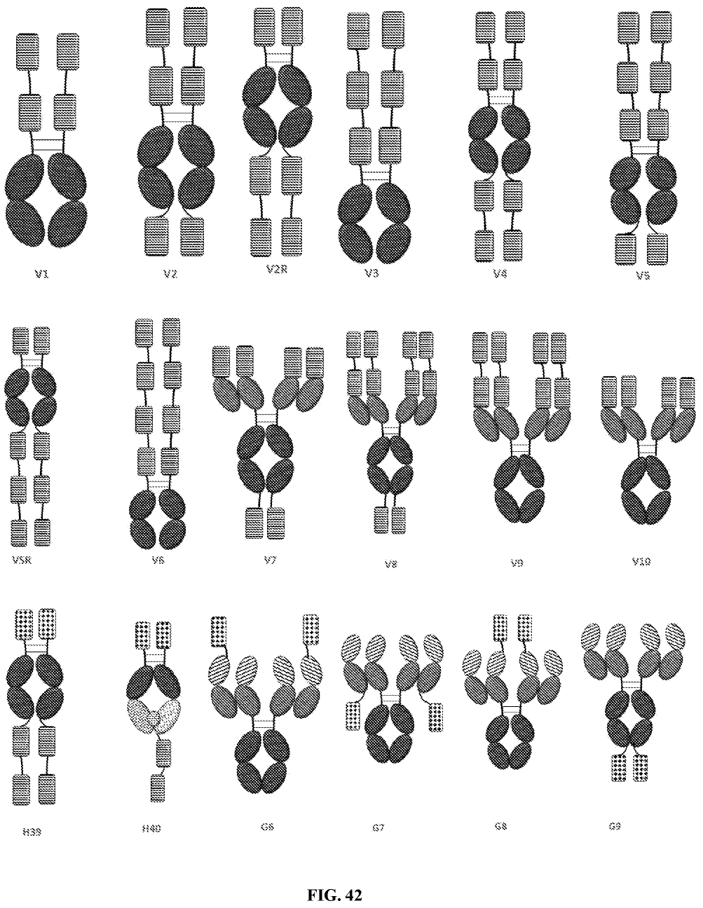

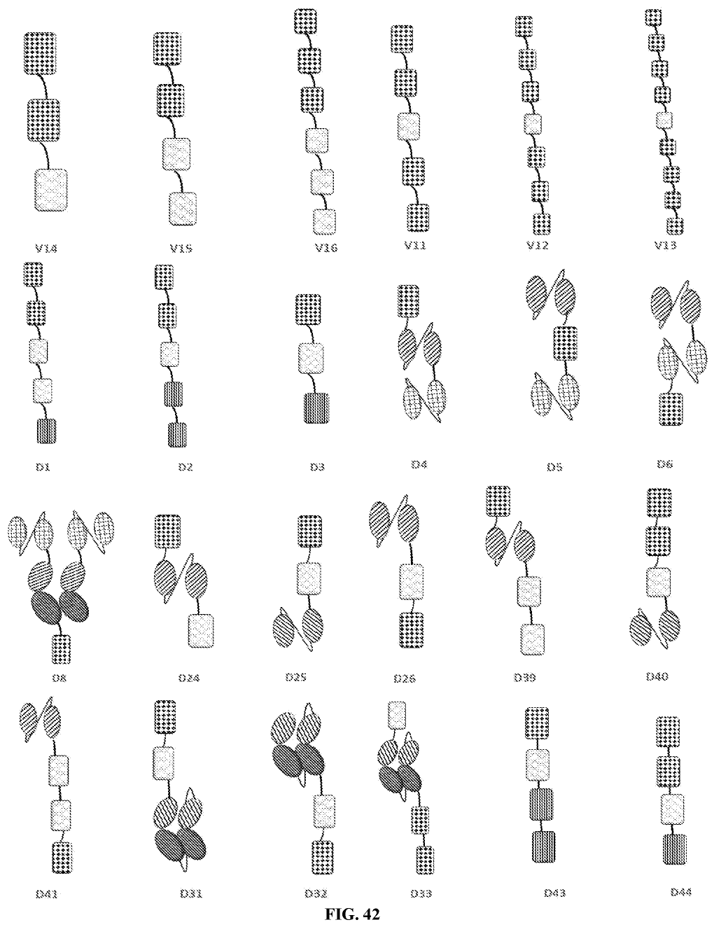

In some embodiments, the antigen binding protein comprises or consists of two polypeptides. The first polypeptide can comprise or consist of, e.g., preferably from N-terminus to C-terminus, a VHH, a VH, and a CH1 domain. The second polypeptide can comprise or consist of, e.g., preferably from N-terminus to C-terminus, a VHH, a VL, and a CL domain. The CH1 domain and the CL domain can associate with each other. In some embodiments, the antigen binding protein comprises or consists of a structure as shown in FIG. 41A. In some embodiments, the VHH in the first polypeptide targets VEGF; the VHH in the second polypeptide targets Ang-2; the VH and the VL associate with each other, forming an antigen-binding site that targets PD-1.

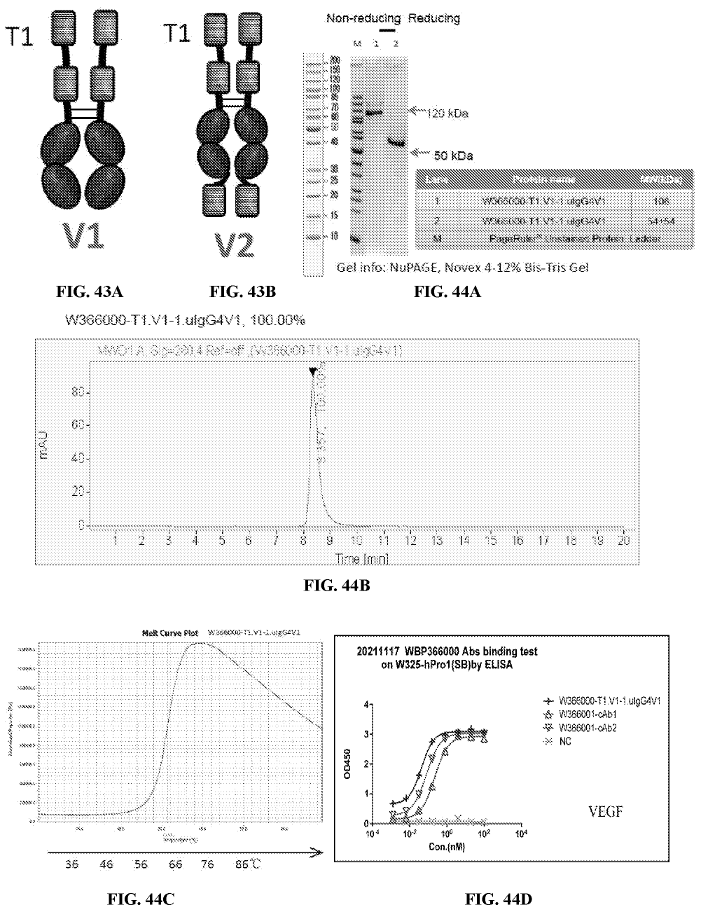

In some embodiments, the antigen binding protein comprises or consists of two polypeptides. The first polypeptide can comprise or consist of, e.g., preferably from N-terminus to C-terminus, two VHHs, an optional hinge region sequence, a CH2 domain and a CH3 domain. In some embodiments, the second polypeptide can comprise or consist of, e.g., preferably from N-terminus to C-terminus, two VHHs, an optional hinge region sequence, a CH2 domain and a CH3 domain. In some embodiments, the antigen binding protein comprises or consists of a structure shown as V1 in FIG. 42 or FIG. 43A.

In some embodiments, the antigen binding protein comprises or consists of two polypeptides. The first polypeptide can comprise or consist of, e.g., preferably from N-terminus to C-terminus, two VHHs, an optional hinge region sequence, a CH2 domain, a CH3 domain and a VHH. In some embodiments, the second polypeptide can comprise or consist of, e.g., preferably from N-terminus to C-terminus, two VHHs, an optional hinge region sequence, a CH2 domain, a CH3 domain, and a VHH. In some embodiments, the antigen binding protein comprises or consists of a structure shown as V2 in FIG. 42 or FIG. 43B.

In some embodiments, the antigen binding protein comprises or consists of two polypeptides. The first polypeptide can comprise or consist of, e.g., preferably from N-terminus to C-terminus, a VHH, an optional hinge region sequence, a CH2 domain, a CH3 domain and two VHHs. In some embodiments, the second polypeptide can comprise or consist of, e.g., preferably from N-terminus to C-terminus, a VHH, an optional hinge region sequence, a CH2 domain, a CH3 domain, and two VHHs. In some embodiments, the antigen binding protein comprises or consists of a structure shown as V2R in FIG. 42.

In some embodiments, the antigen binding protein comprises or consists of two polypeptides. The first polypeptide can comprise or consist of, e.g., preferably from N-terminus to C-terminus, three VHHs, an optional hinge region sequence, a CH2 domain and a CH3 domain. In some embodiments, the second polypeptide can comprise or consist of, e.g., preferably from N-terminus to C-terminus, three VHHs, an optional hinge region sequence, a CH2 domain and a CH3 domain. In some embodiments, the antigen binding protein comprises or consists of a structure shown as V3 in FIG. 42.

In some embodiments, the antigen binding protein comprises or consists of two polypeptides. The first polypeptide can comprise or consist of, e.g., preferably from N-terminus to C-terminus, two VHHs, an optional hinge region sequence, a CH2 domain, a CH3 domain and two VHHs. In some embodiments, the second polypeptide can comprise or consist of, e.g., preferably from N-terminus to C-terminus, two VHHs, an optional hinge region sequence, a CH2 domain, a CH3 domain, and two VHHs. In some embodiments, the antigen binding protein comprises or consists of a structure shown as V4 in FIG. 42.

In some embodiments, the antigen binding protein comprises or consists of two polypeptides. The first polypeptide can comprise or consist of, e.g., preferably from N-terminus to C-terminus, three VHHs, an optional hinge region sequence, a CH2 domain, a CH3 domain and a VHH. In some embodiments, the second polypeptide can comprise or consist of, e.g., preferably from N-terminus to C-terminus, three VHHs, an optional hinge region sequence, a CH2 domain, a CH3 domain, and a VHH. In some embodiments, the antigen binding protein comprises or consists of a structure shown as V5 in FIG. 42.

In some embodiments, the antigen binding protein comprises or consists of two polypeptides. The first polypeptide can comprise or consist of, e.g., preferably from N-terminus to C-terminus, a VHH, an optional hinge region sequence, a CH2 domain, a CH3 domain and three VHHs. In some embodiments, the second polypeptide can comprise or consist of, e.g., preferably from N-terminus to C-terminus, a VHH, an optional hinge region sequence, a CH2 domain, a CH3 domain, and three VHHs. In some embodiments, the antigen binding protein comprises or consists of a structure shown as V5R in FIG. 42.

In some embodiments, the antigen binding protein comprises or consists of two polypeptides. The first polypeptide can comprise or consist of, e.g., preferably from N-terminus to C-terminus, four VHHs, an optional hinge region sequence, a CH2 domain and a CH3 domain. In some embodiments, the second polypeptide can comprise or consist of, e.g., preferably from N-terminus to C-terminus, four VHHs, an optional hinge region sequence, a CH2 domain and a CH3 domain. In some embodiments, the antigen binding protein comprises or consists of a structure shown as V6 in FIG. 42.

In some embodiments, the antigen binding protein comprises or consists of four polypeptides. The first polypeptide can comprise or consist of, e.g., preferably from N-terminus to C-terminus, a VHH, a CH1 domain, an optional hinge region sequence, a CH2 domain, a CH3 domain and a second VHH. The second polypeptide can comprise or consist of, e.g., preferably from N-terminus to C-terminus, a VHH, a CH1 domain, an optional hinge region sequence, a CH2 domain, a CH3 domain, and a second VHH. The third polypeptide can comprise or consist of, e.g., preferably from N-terminus to C-terminus, a VHH and a CL domain. The fourth polypeptide can comprise or consist of, e.g., preferably from N-terminus to C-terminus, a VHH and a CL domain. The CH1 domain in the first polypeptide and the CL domain in the third polypeptide can associate with each other. The CH1 domain in the second polypeptide and the CL domain in the fourth polypeptide can associate with each other. In some embodiments, the antigen binding protein comprises or consists of a structure shown as V7 in FIG. 42.

In some embodiments, the antigen binding protein comprises or consists of four polypeptides. The first polypeptide can comprise or consist of, e.g., preferably from N-terminus to C-terminus, two VHHs, a CH1 domain, an optional hinge region sequence, a CH2 domain, a CH3 domain and a second VHH. The second polypeptide can comprise or consist of, e.g., preferably from N-terminus to C-terminus, two VHHs, a CH1 domain, an optional hinge region sequence, a CH2 domain, a CH3 domain, and a second VHH. The third polypeptide can comprise or consist of, e.g., preferably from N-terminus to C-terminus, two VHHs and a CL domain. The fourth polypeptide can comprise or consist of, e.g., preferably from N-terminus to C-terminus, two VHHs and a CL domain. The CH1 domain in the first polypeptide and the CL domain in the third polypeptide can associate with each other. The CH1 domain in the second polypeptide and the CL domain in the fourth polypeptide can associate with each other. In some embodiments, the antigen binding protein comprises or consists of a structure shown as V8 in FIG. 42.

In some embodiments, the antigen binding protein comprises or consists of four polypeptides. The first polypeptide can comprise or consist of, e.g., preferably from N-terminus to C-terminus, two VHHs, a CH1 domain, an optional hinge region sequence, a CH2 domain, and a CH3 domain. The second polypeptide can comprise or consist of, e.g., preferably from N-terminus to C-terminus, two VHHs, a CH1 domain, an optional hinge region sequence, a CH2 domain, and a CH3 domain. The third polypeptide can comprise or consist of, e.g., preferably from N-terminus to C-terminus, two VHHs and a CL domain. The fourth polypeptide can comprise or consist of, e.g., preferably from N-terminus to C-terminus, two VHHs and a CL domain. The CH1 domain in the first polypeptide and the CL domain in the third polypeptide can associate with each other. The CH1 domain in the second polypeptide and the CL domain in the fourth polypeptide can associate with each other. In some embodiments, the antigen binding protein comprises or consists of a structure shown as V9 in FIG. 42.

In some embodiments, the antigen binding protein comprises or consists of four polypeptides. The first polypeptide can comprise or consist of, e.g., preferably from N-terminus to C-terminus, a VHH, a CH1 domain, an optional hinge region sequence, a CH2 domain, and a CH3 domain. The second polypeptide can comprise or consist of, e.g., preferably from N-terminus to C-terminus, a VHH, a CH1 domain, an optional hinge region sequence, a CH2 domain, and a CH3 domain. The third polypeptide can comprise or consist of, e.g., preferably from N-terminus to C-terminus, a VHH and a CL domain. The fourth polypeptide can comprise or consist of, e.g., preferably from N-terminus to C-terminus, a VHH and a CL domain. The CH1 domain in the first polypeptide and the CL domain in the third polypeptide can associate with each other. The CH1 domain in the second polypeptide and the CL domain in the fourth polypeptide can associate with each other. In some embodiments, the antigen binding protein comprises or consists of a structure shown as V10 in FIG. 42.

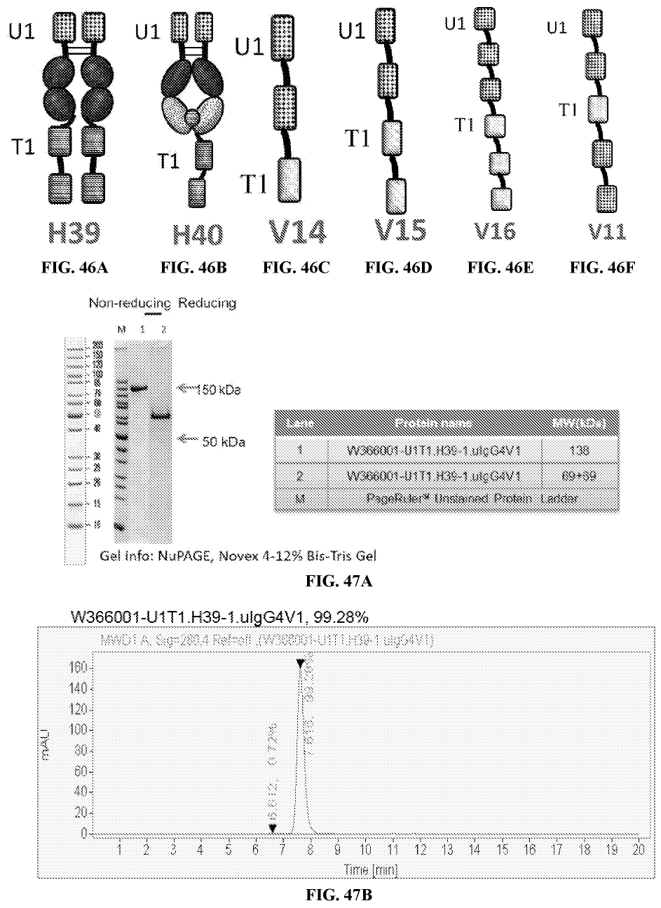

In some embodiments, the antigen binding protein comprises or consists of two polypeptides. The first polypeptide can comprise or consist of, e.g., preferably from N-terminus to C-terminus, a first VHH, an optional hinge region sequence, a CH2 domain, a CH3 domain, and two second VHHs. In some embodiments, the second polypeptide can comprise or consist of, e.g., preferably from N-terminus to C-terminus, a first VHH, an optional hinge region sequence, a CH2 domain, a CH3 domain, and two second VHHs. In some embodiments, the first VHH and the second VHH are different. In some embodiments, the first VHH and the second VHH are the same. In some embodiments, the antigen binding protein comprises or consists of a structure shown as H39 in FIG. 42 or FIG. 46A.

In some embodiments, the antigen binding protein comprises or consists of two polypeptides. The first polypeptide can comprise or consist of, e.g., preferably from N-terminus to C-terminus, a first VHH, an optional hinge region sequence, a CH2 domain, and a CH3 domain. In some embodiments, the second polypeptide can comprise or consist of, e.g., preferably from N-terminus to C-terminus, a first VHH, an optional hinge region sequence, a CH2 domain, a CH3 domain, and two second VHHs. In some embodiments, the first VHH and the second VHH are different. In some embodiments, the first VHH and the second VHH are the same. In some embodiments, KIH mutations are introduced (e.g., in the CH3 domains). In some embodiments, the antigen binding protein comprises or consists of a structure shown as H40 in FIG. 42 or FIG. 46B.

In some embodiments, the antigen binding protein comprises or consists of four polypeptides. The first polypeptide can comprise or consist of, e.g., preferably from N-terminus to C-terminus, a first VH, a CH1 domain, an optional hinge region sequence, a CH2 domain, and a CH3 domain. The second polypeptide can comprise or consist of, e.g., preferably from N-terminus to C-terminus, a second VH, a CH1 domain, an optional hinge region sequence, a CH2 domain, and a CH3 domain. The third polypeptide can comprise or consist of, e.g., preferably from N-terminus to C-terminus, a VHH, a first VL, and a CL domain. The fourth polypeptide can comprise or consist of, e.g., preferably from N-terminus to C-terminus, a VHH, a second VL, and a CL domain. The CH1 domain in the first polypeptide and the CL domain in the third polypeptide can associate with each other. The CH1 domain in the second polypeptide and the CL domain in the fourth polypeptide can associate with each other. The first VH in the first polypeptide and the first VL in the third polypeptide can associate with each other, forming a first antigen-binding site. The second VH in the second polypeptide and the second VL in the fourth polypeptide can associate with each other, forming a second antigen-binding site. In some embodiments, the VHH in the third polypeptide and the VHH in the fourth polypeptide are the same. In some embodiments, the VHH in the third polypeptide and the VHH in the fourth polypeptide are the different. In some embodiments, the first antigen-binding site and the second antigen-binding site target the same antigen. In some embodiments, the first antigen-binding site and the second antigen-binding site target different antigens. In some embodiments, the antigen binding protein comprises or consists of a structure shown as G6 in FIG. 42.

In some embodiments, the antigen binding protein comprises or consists of four polypeptides. The first polypeptide can comprise or consist of, e.g., preferably from N-terminus to C-terminus, a first VH, a CH1 domain, an optional hinge region sequence, a CH2 domain, and a CH3 domain. The second polypeptide can comprise or consist of, e.g., preferably from N-terminus to C-terminus, a second VH, a CH1 domain, an optional hinge region sequence, a CH2 domain, and a CH3 domain. The third polypeptide can comprise or consist of, e.g., preferably from N-terminus to C-terminus, a first VL, a CL domain, and a VHH. The fourth polypeptide can comprise or consist of, e.g., preferably from N-terminus to C-terminus, a second VL, a CL domain, and a VHH. The CH1 domain in the first polypeptide and the CL domain in the third polypeptide can associate with each other. The CH1 domain in the second polypeptide and the CL domain in the fourth polypeptide can associate with each other. The first VH in the first polypeptide and the first VL in the third polypeptide can associate with each other, forming a first antigen-binding site. The second VH in the second polypeptide and the second VL in the fourth polypeptide can associate with each other, forming a second antigen-binding site. In some embodiments, the VHH in the third polypeptide and the VHH in the fourth polypeptide are the same. In some embodiments, the VHH in the third polypeptide and the VHH in the fourth polypeptide are different. In some embodiments, the first antigen-binding site and the second antigen-binding site target the same antigen. In some embodiments, the first antigen-binding site and the second antigen-binding site target different antigens. In some embodiments, the antigen binding protein comprises or consists of a structure shown as G7 in FIG. 42.

In some embodiments, the antigen binding protein comprises or consists of four polypeptides. The first polypeptide can comprise or consist of, e.g., preferably from N-terminus to C-terminus, a VHH, a first VH, a CH1 domain, an optional hinge region sequence, a CH2 domain, and a CH3 domain. The second polypeptide can comprise or consist of, e.g., preferably from N-terminus to C-terminus, a VHH, a second VH, a CH1 domain, an optional hinge region sequence, a CH2 domain, and a CH3 domain. The third polypeptide can comprise or consist of, e.g., preferably from N-terminus to C-terminus, a first VL and a CL domain. The fourth polypeptide can comprise or consist of, e.g., preferably from N-terminus to C-terminus, a second VL and a CL domain. The CH1 domain in the first polypeptide and the CL domain in the third polypeptide can associate with each other. The CH1 domain in the second polypeptide and the CL domain in the fourth polypeptide can associate with each other. The first VH in the first polypeptide and the first VL in the third polypeptide can associate with each other, forming a first antigen-binding site. The second VH in the second polypeptide and the second VL in the fourth polypeptide can associate with each other, forming a second antigen-binding site. In some embodiments, the VHH in the first polypeptide and the VHH in the second polypeptide are the same. In some embodiments, the VHH in the first polypeptide and the VHH in the second polypeptide are different. In some embodiments, the first antigen-binding site and the second antigen-binding site target the same antigen. In some embodiments, the first antigen-binding site and the second antigen-binding site target different antigens. In some embodiments, the antigen binding protein comprises or consists of a structure shown as G8 in FIG. 42.

In some embodiments, the antigen binding protein comprises or consists of four polypeptides. The first polypeptide can comprise or consist of, e.g., preferably from N-terminus to C-terminus, a first VH, a CH1 domain, an optional hinge region sequence, a CH2 domain, a CH3 domain, and a VHH. The second polypeptide can comprise or consist of, e.g., preferably from N-terminus to C-terminus, a second VH, a CH1 domain, an optional hinge region sequence, a CH2 domain, a CH3 domain, and a VHH. The third polypeptide can comprise or consist of, e.g., preferably from N-terminus to C-terminus, a first VL and a CL domain. The fourth polypeptide can comprise or consist of, e.g., preferably from N-terminus to C-terminus, a second VL and a CL domain. The CH1 domain in the first polypeptide and the CL domain in the third polypeptide can associate with each other. The CH1 domain in the second polypeptide and the CL domain in the fourth polypeptide can associate with each other. The first VH in the first polypeptide and the first VL in the third polypeptide can associate with each other, forming a first antigen-binding site. The second VH in the second polypeptide and the second VL in the fourth polypeptide can associate with each other, forming a second antigen-binding site. In some embodiments, the VHH in the first polypeptide and the VHH in the second polypeptide are the same. In some embodiments, the VHH in the first polypeptide and the VHH in the second polypeptide are different. In some embodiments, the first antigen-binding site and the second antigen-binding site target the same antigen. In some embodiments, the first antigen-binding site and the second antigen-binding site target different antigens. In some embodiments, the antigen binding protein comprises or consists of a structure shown as G9 in FIG. 42.

In some embodiments, the antigen binding protein comprises or consists of a single polypeptide. The polypeptide can comprise or consist of, e.g., preferably from N-terminus to C-terminus, two first VHHs and a second VHH. In some embodiments, the first VHH and the second VHH are different. In some embodiments, the first VHH and the second VHH are the same. In some embodiments, the antigen binding protein comprises or consists of a structure shown as V14 in FIG. 42 or FIG. 46C.

In some embodiments, the antigen binding protein comprises or consists of a single polypeptide. The polypeptide can comprise or consist of, e.g., preferably from N-terminus to C-terminus, two first VHHs and two second VHHs. In some embodiments, the first VHH and the second VHH are different. In some embodiments, the first VHH and the second VHH are the same. In some embodiments, the antigen binding protein comprises or consists of a structure shown as V15 in FIG. 42 or FIG. 46D.

In some embodiments, the antigen binding protein comprises or consists of a single polypeptide. The polypeptide can comprise or consist of, e.g., preferably from N-terminus to C-terminus, three first VHHs and three second VHHs. In some embodiments, the first VHH and the second VHH are different. In some embodiments, the first VHH and the second VHH are the same. In some embodiments, the antigen binding protein comprises or consists of a structure shown as V16 in FIG. 42 or FIG. 46E.

In some embodiments, the antigen binding protein comprises or consists of a single polypeptide. The polypeptide can comprise or consist of, e.g., preferably from N-terminus to C-terminus, two first VHHs, a second VHH, and two first VHHs. In some embodiments, the first VHH and the second VHH are different. In some embodiments, the first VHH and the second VHH are the same. In some embodiments, the antigen binding protein comprises or consists of a structure shown as V11 in FIG. 42 or FIG. 46F.

In some embodiments, the antigen binding protein comprises or consists of a single polypeptide. The polypeptide can comprise or consist of, e.g., preferably from N-terminus to C-terminus, three first VHHs, a second VHH, and three first VHHs. In some embodiments, the first VHH and the second VHH are different. In some embodiments, the first VHH and the second VHH are the same. In some embodiments, the antigen binding protein comprises or consists of a structure shown as V12 in FIG. 42.

In some embodiments, the antigen binding protein comprises or consists of a single polypeptide. The polypeptide can comprise or consist of, e.g., preferably from N-terminus to C-terminus, four first VHHs, a second VHH, and four first VHHs. In some embodiments, the first VHH and the second VHH are different. In some embodiments, the first VHH and the second VHH are the same. In some embodiments, the antigen binding protein comprises or consists of a structure shown as V13 in FIG. 42.

In some embodiments, the antigen binding protein comprises or consists of a single polypeptide. The polypeptide can comprise or consist of, e.g., preferably from N-terminus to C-terminus, two first VHHs, two second VHHs, and a third VHH. In some embodiments, the first VHH, the second VHH, and the third VHH are different from each other. In some embodiments, two out of the first VHH, the second VHH, and the third VHH are the same. In some embodiments, all three of the first VHH, the second VHH, and the third VHH are the same. In some embodiments, the antigen binding protein comprises or consists of a structure shown as D1 in FIG. 42 or FIG. 53A.

In some embodiments, the antigen binding protein comprises or consists of a single polypeptide. The polypeptide can comprise or consist of, e.g., preferably from N-terminus to C-terminus, two first VHHs, a second VHH, and two third VHHs. In some embodiments, the first VHH, the second VHH, and the third VHH are different from each other. In some embodiments, two out of the first VHH, the second VHH, and the third VHH are the same. In some embodiments, all three of the first VHH, the second VHH, and the third VHH are the same. In some embodiments, the antigen binding protein comprises or consists of a structure shown as D2 in FIG. 42 or FIG. 53B.

In some embodiments, the antigen binding protein comprises or consists of a single polypeptide. The polypeptide can comprise or consist of, e.g., preferably from N-terminus to C-terminus, a first VHH, a second VHH, and a third VHH. In some embodiments, the first VHH, the second VHH, and the third VHH are different from each other. In some embodiments, two out of the first VHH, the second VHH, and the third VHH are the same. In some embodiments, all three of the first VHH, the second VHH, and the third VHH are the same. In some embodiments, the antigen binding protein comprises or consists of a structure shown as D3 in FIG. 42 or FIG. 53C.

In some embodiments, the antigen binding protein comprises or consists of a single polypeptide. The polypeptide can comprise or consist of, e.g., preferably from N-terminus to C-terminus, a VHH, a first scFv, and a second scFv. In some embodiments, the first scFv and the second scFv are different. In some embodiments, the first scFv and the second scFv are the same. In some embodiments, the antigen binding protein comprises or consists of a structure shown as D4 in FIG. 42.

In some embodiments, the antigen binding protein comprises or consists of a single polypeptide. The polypeptide can comprise or consist of, e.g., preferably from N-terminus to C-terminus, a first scFv, a VHH, and a second scFv. In some embodiments, the first scFv and the second scFv are different. In some embodiments, the first scFv and the second scFv are the same. In some embodiments, the antigen binding protein comprises or consists of a structure shown as D5 in FIG. 42.

In some embodiments, the antigen binding protein comprises or consists of a single polypeptide. The polypeptide can comprise or consist of, e.g., preferably from N-terminus to C-terminus, a first scFv, a second scFv, and a VHH. In some embodiments, the first scFv and the second scFv are different. In some embodiments, the first scFv and the second scFv are the same. In some embodiments, the antigen binding protein comprises or consists of a structure shown as D6 in FIG. 42.

In some embodiments, the antigen binding protein comprises or consists of two polypeptides. In some embodiments, the first polypeptide can comprise or consist of, e.g., preferably from N-terminus to C-terminus, a scFv, a VH, a CH1 domain, and a VHH; and the second polypeptide can comprise or consist of, e.g., preferably from N-terminus to C-terminus, a scFv, a VL, and a CL domain. In some embodiments, the first polypeptide can comprise or consist of, e.g., preferably from N-terminus to C-terminus, a scFv, a VL, a CL domain, and a VHH; and the second polypeptide can comprise or consist of, e.g., preferably from N-terminus to C-terminus, a scFv, a VH, and a CH1 domain. The CH1 domain and the CL domain can associate with each other. In some embodiments, the VH and the VL can associate with each other, forming an antigen-binding site. In some embodiments, the scFv in the first polypeptide and the scFv in the second polypeptide are the same. In some embodiments, the scFv in the first polypeptide and the scFv in the second polypeptide are different. In some embodiments, the antigen binding protein comprises or consists of a structure shown as D8 in FIG. 42.

In some embodiments, the antigen binding protein comprises or consists of a single polypeptide. The polypeptide can comprise or consist of, e.g., preferably from N-terminus to C-terminus, a first VHH, a scFv, and a second VHH. In some embodiments, the first VHH and the second VHH are different. In some embodiments, the first VHH and the second VHH are the same. In some embodiments, the antigen binding protein comprises or consists of a structure shown as D24 in FIG. 42.

In some embodiments, the antigen binding protein comprises or consists of a single polypeptide. The polypeptide can comprise or consist of, e.g., preferably from N-terminus to C-terminus, a first VHH, a second VHH, and a scFv. In some embodiments, the first VHH and the second VHH are different. In some embodiments, the first VHH and the second VHH are the same. In some embodiments, the antigen binding protein comprises or consists of a structure shown as D25 in FIG. 42.

In some embodiments, the antigen binding protein comprises or consists of a single polypeptide. The polypeptide can comprise or consist of, e.g., preferably from N-terminus to C-terminus, a scFv, a first VHH, and a second VHH. In some embodiments, the first VHH and the second VHH are different. In some embodiments, the first VHH and the second VHH are the same. In some embodiments, the antigen binding protein comprises or consists of a structure shown as D26 in FIG. 42.

In some embodiments, the antigen binding protein comprises or consists of a single polypeptide. The polypeptide can comprise or consist of, e.g., preferably from N-terminus to C-terminus, a first VHH, a scFv, and two second VHHs. In some embodiments, the first VHH and the second VHH are different. In some embodiments, the first VHH and the second VHH are the same. In some embodiments, the antigen binding protein comprises or consists of a structure shown as D39 in FIG. 42.

In some embodiments, the antigen binding protein comprises or consists of a single polypeptide. The polypeptide can comprise or consist of, e.g., preferably from N-terminus to C-terminus, two first VHHs, a second VHH, and a scFv. In some embodiments, the first VHH and the second VHH are different. In some embodiments, the first VHH and the second VHH are the same. In some embodiments, the antigen binding protein comprises or consists of a structure shown as D40 in FIG. 42.

In some embodiments, the antigen binding protein comprises or consists of a single polypeptide. The polypeptide can comprise or consist of, e.g., preferably from N-terminus to C-terminus, a scFv, two first VHHs, and a second VHH. In some embodiments, the first VHH and the second VHH are different. In some embodiments, the first VHH and the second VHH are the same. In some embodiments, the antigen binding protein comprises or consists of a structure shown as D41 in FIG. 42.

In some embodiments, the antigen binding protein comprises or consists of a single polypeptide. In some embodiments, the polypeptide can comprise or consist of, e.g., preferably from N-terminus to C-terminus, a first VHH, a second VHH, a VH, a CH1 domain, a VL, and a CL domain. In some embodiments, the polypeptide can comprise or consist of, e.g., preferably from N-terminus to C-terminus, a first VHH, a second VHH, a VL, a CL domain, a VH, and a CH1 domain. The CH1 domain and the CL domain can associate with each other. In some embodiments, the first VHH and the second VHH are different. In some embodiments, the first VHH and the second VHH are the same. In some embodiments, the antigen binding protein comprises or consists of a structure shown as D31 in FIG. 42.

In some embodiments, the antigen binding protein comprises or consists of a single polypeptide. In some embodiments, the polypeptide can comprise or consist of, e.g., preferably from N-terminus to C-terminus, a VH, a CH1 domain, a VL, a CL domain, a first VHH, and a second VHH. In some embodiments, the polypeptide can comprise or consist of, e.g., preferably from N-terminus to C-terminus, a VL, a CL domain, a VH, a CH1 domain, a first VHH, and a second VHH. The CH1 domain and the CL domain can associate with each other. In some embodiments, the first VHH and the second VHH are different. In some embodiments, the first VHH and the second VHH are the same. In some embodiments, the antigen binding protein comprises or consists of a structure shown as D32 in FIG. 42.

In some embodiments, the antigen binding protein comprises or consists of a single polypeptide. In some embodiments, the polypeptide can comprise or consist of, e.g., preferably from N-terminus to C-terminus, a first VHH, a VH, a CH1 domain, a VL, a CL domain, and two second VHHs. In some embodiments, the polypeptide can comprise or consist of, e.g., preferably from N-terminus to C-terminus, a first VHH, a VL, a CL domain, a VH, a CH1 domain, and two second VHHs. The CH1 domain and the CL domain can associate with each other. In some embodiments, the first VHH and the second VHH are different. In some embodiments, the first VHH and the second VHH are the same. In some embodiments, the antigen binding protein comprises or consists of a structure shown as D33 in FIG. 42.

In some embodiments, the antigen binding protein comprises or consists of a single polypeptide. The polypeptide can comprise or consist of, e.g., preferably from N-terminus to C-terminus, a first VHH, a second VHH, and two third VHHs. In some embodiments, the first VHH, the second VHH, and the third VHH are different from each other. In some embodiments, two out of the first VHH, the second VHH, and the third VHH are the same. In some embodiments, all three of the first VHH, the second VHH, and the third VHH are the same. In some embodiments, the antigen binding protein comprises or consists of a structure shown as D43 in FIG. 42 or FIG. 53D.

In some embodiments, the antigen binding protein comprises or consists of a single polypeptide. The polypeptide can comprise or consist of, e.g., preferably from N-terminus to C-terminus, two first VHHs, a second VHH, and a third VHH. In some embodiments, the first VHH, the second VHH, and the third VHH are different from each other. In some embodiments, two out of the first VHH, the second VHH, and the third VHH are the same. In some embodiments, all three of the first VHH, the second VHH, and the third VHH are the same. In some embodiments, the antigen binding protein comprises or consists of a structure shown as D44 in FIG. 42 or FIG. 53E.

In some embodiments, one or more additional VHHs can be added to the antigen binding protein as described herein. In some embodiments, one or more additional Fab domains (including e.g., a VH-CH1 that is associated with a VL-CL) can be added to the antigen binding protein as described herein.

In some embodiments, the VHH, the VH, or the VL can be linked to the N terminal or C terminal of a polypeptide through a linker peptide sequence. The linker peptide sequence can be the same or different. Each peptide linker sequence can be optimized individually. The linker sequence can have at least or about 1, 2, 3, 4, 5, 6, 7, 8, 9, 10, 11, 12, 13, 14, 15, 16, 17, 18, 19, 20, 25, 30, 35, 40, or 50 amino acids. In some embodiments, the peptide linker has no more than 50, 40, 35, 30, 25, 20, 19, 18, 17, 16, 15, 14, 13, 12, 11, 10, 9, 8, 7, 6, 5 or fewer amino acids. In some embodiments, the peptide linker has no more than about 30 (such as no more than about any one of 25, 20, or 15) amino acids. In some embodiments, the length of the peptide linker is about 1-30, 1-20, 5-30, 5-20, or 5-10 amino acids.

The peptide linker can have a naturally occurring sequence, or a non-naturally occurring sequence. For example, a sequence derived from the hinge region of heavy chain can be used as the linker. These sequences are described in WO1996/34103, which is incorporated herein by reference in the entirety. In some embodiments, the peptide linker is a flexible linker. Exemplary flexible linkers include glycine polymers (G) n, glycine-serine polymers (including, for example, (GS)n, (GSGGS)n (SEQ ID NO: 1) and (GGGS)n (SEQ ID NO: 2), where n is an integer of at least or about 1, 2, 3, 4, or 5), glycine-alanine polymers, alanine-serine polymers, and other flexible linkers known in the art. In some embodiments, the peptide linker comprises the amino acid sequence GGGGS (SEQ ID NO: 3) or GGGGSGGGGS (SEQ ID NO: 4). In some embodiments, the peptide linker comprises the hinge region of an IgG, such as the hinge region of human IgG1. In some embodiments, the peptide linker comprises the amino acid sequence EPKSCDKTHTCPPCP (SEQ ID NO: 5). In some embodiments, the peptide linker comprises a modified sequence derived from the hinge region of an IgG, such as the hinge region of human IgG1. For example, one or more cysteine amino acids in the hinge region of an IgG may be replaced with a serine. In some embodiments, the peptide linker comprises the amino acid sequence EPKSSDKTHTSPPSP (SEQ ID NO: 6).

### Antigen Binding Sites

The antigen binding proteins can comprise or consist of 1, 2, 3, 4, 5, 6, 7, 8, 9, 10, 11, 12 or more than 12 antigen binding sites or antigen binding portions. An antigen binding site refers to a functional structure in a protein that can specifically bind to an antigen. The antigen binding site can be formed by a pair of VH and VL. Alternative, the antigen binding site can be formed by a VHH.

The antigen binding proteins as described herein can bind to 1, 2, 3, 4, 5, 6, 7, 8, 9, 10, 11, 12 or more than 12 epitopes or antigens. These epitopes can be the same or different. In some embodiments, these epitopes can be different but they are in the same antigen. In some embodiments, the antigen binding proteins as described herein can bind to 1, 2, 3, 4, 5, 6, 7, 8, 9, 10, 11, 12 or more than 12 different antigens.

In some embodiments, the antigen binding proteins can bind to one or more immune checkpoint molecules. These immune checkpoint molecules are regulators of the immune system. In some embodiments, the immune checkpoint molecules include e.g., programmed cell death protein 1 (PD-1), TNF Receptor Superfamily Member 9 (4-1BB or CD137), cytotoxic T-lymphocyte-associated protein 4 (CTLA-4), LAG-3, T-cell immunoglobulin and mucin-domain containing-3 (TIM-3), B And T Lymphocyte Associated (BTLA), Programmed Cell Death 1 Ligand 1 (PD-L1), CD27, CD28, CD40, CD47, CD122, ICOS, T-Cell Immunoreceptor With Ig And ITIM Domains (TIGIT), Glucocorticoid-Induced TNFR-Related Protein (GITR), A2AR, CD278, VTCN1, BTLA, IDO, KIR, NOX2, VISTA, SIGLEC7, or TNF Receptor Superfamily Member 4 (TNFRSF4; or OX40).

In some embodiments, the antigen binding proteins can bind to one or more cancer specific antigens. As used herein, the term “cancer specific antigen” refers to antigens that are specifically expressed on cancer cell surfaces. These antigens can be used to identify tumor cells. Normal cells rarely express cancer specific antigens. Some exemplary cancer specific antigens include, e.g., CD20, PSA, PSCA, PD-L1, Her2, Her3, Her1, β-Catenin, CD19, CEACAM3, EGFR, c-Met, EPCAM, PSMA, CD40, MUC1, and IGF1R, etc.

In some embodiments, the antigen binding proteins can bind to one or more cancer cancer-associated antigens. As used herein, the term “cancer-associated antigen” refers to antigens that are expressed at a relatively high level on cancer cells but may be also expressed at a relatively low level on normal cells. CD55, CD59, CD46 and many adhesion molecules such as N-cadherin, VE-cadherin, NCAM, Mel-CAM, ICAM, NrCAM, VCAM1, ALCAM, MCAM, etc., are cancer-associated antigens. While both cancer specific antigen and cancer-associated antigen are expressed on cancer cell surface, the difference between a cancer specific antigen and a cancer-associated antigen is that the cancer-associated antigen is also expressed on normal cells, but at a relative low level as compared to the level on cancer cells. In contrast, a cancer specific antigen is rarely expressed on normal cells, and even if it is expressed on normal cells, the amount is extremely low.

In some embodiments, the cancer antigens include, e.g., a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

Non-limiting examples of antigens the antigen binding proteins can bind to further include, e.g., differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

In some embodiments, the cell surface antigen is an antigen on immune effector cells, such as T cells (e.g., helper T cells, cytotoxic T cells, memory T cells), B cells, macrophages, and natural killer (NK) cells. In some embodiments, the cell surface antigen is a T cell surface antigen, e.g., CD3.

In some embodiments, the antigen binding proteins binds to two or more antigens selected from the group consisting of VEGF, Ang-2, MSLN, GITR, and PD-1.

In some embodiments, the antigen binding proteins (e.g., bispecific antibody) can recruit a tumor cell to an immune cell (e.g., T cell) and activate the immune cells. In some embodiments, the antigen binding proteins can increase the immune response.

In some embodiments, the antigen binding proteins are designed that include an additional antigen binding region that targets a cancer antigen.

In some embodiments, the antigen binding proteins can have the antigen binding site derived from, or bind to the same epitope or antigen of various therapeutic antibodies, including e.g., Adalimumab, Bezlotoxumab, Avelumab, Dupilumab, Durvalumab, Ocrelizumab, Brodalumab, Reslizumab, Olaratumab, Daratumumab, Elotuzumab, Necitumumab, Infliximab, Obiltoxaximab, Atezolizumab, Secukinumab, Mepolizumab, Nivolumab, Alirocumab, Evolocumab, Dinutuximab, Bevacizumab, Pembrolizumab, Ramucirumab, Vedolizumab, Siltuximab, Alemtuzumab, Trastuzumab, Pertuzumab, Obinutuzumab, Brentuximab, Raxibacumab, Belimumab, Ipilimumab, Denosumab, Ofatumumab, Besilesomab, Tocilizumab, Canakinumab, Golimumab, Ustekinumab, Certolizumab, Catumaxomab, Eculizumab, Ranibizumab, Panitumumab, Natalizumab, Omalizumab, Cetuximab, Efalizumab, Ibritumomab, Fanolesomab, Tositumomab, Gemtuzumab, Palivizumab, Necitumumab, Basiliximab, Rituximab, Capromab, Satumomab, and Muromonab.

The disclosure provides e.g., anti-VEGF antibodies, anti-ANG-2 antibodies, anti-MSLN antibodies, anti-GITR antibodies, anti-PD-1 antibodies, the modified antibodies thereof, the chimeric antibodies thereof, and the humanized antibodies thereof. The antigen-binding portions of these antibodies can be used in various antigen binding protein formats as described herein.

In some embodiments, the antigen binding protein comprises one or more VHHs that specifically binds to VEGF. VHHs that specifically bind to VEGF are known in the art, and are described e.g., in US20170247475A1, which is incorporated herein by reference in its entirety.

In some embodiments, the antigen binding protein comprises one or more VHHs that specifically binds to Ang-2. VHHs that specifically bind to Ang-2 are known in the art, and are described e.g., in US20190135907A1, which is incorporated herein by reference in its entirety.

In some embodiments, the antigen binding protein comprises one or more VHHs that specifically binds to MSLN. VHHs that specifically bind to MSLN are known in the art, and are described e.g., in AU2018/265860 A1 or US20180327508A1, which is incorporated herein by reference in its entirety.

In some embodiments, the antigen binding protein comprises one or more VHHs that specifically binds to GITR. VHHs that specifically bind to GITR are known in the art, and are described e.g., in U.S. Ser. No. 10/093,742B2, which is incorporated herein by reference in its entirety.

In some embodiments, the antigen binding protein comprises one or more antigen binding sites that specifically binds to PD-1. Antigen binding fragments that specifically bind to PD-1 are known in the art, and are described e.g., in US2012135408 or U.S. Pat. No. 8,952,136B2, which is incorporated herein by reference in its entirety.

### Characteristics of Antigen Binding Proteins

The antigen binding proteins ad described herein can include an antigen binding site that is derived from any antibodies (e.g., parental antibodies), or any antigen-binding fragment thereof as described herein.

In some embodiments, the antigen binding proteins, or antigen-binding fragments thereof described herein can bind to cells expressing various antigens as described here (e.g., VEGF, Ang-2, PD-1, MSLN, or GITR).

In some embodiments, the antigen binding proteins as described herein can increase immune response. In some embodiments, the antigen binding proteins as described herein can increase immune response, activity or number of T cells (e.g., CD3+ cells, CD8+ and/or CD4+ cells) by at least 10%, 20%, 30%, 40%, 50%, 60%, 70%, 80%, 90%, 100%, 2 folds, 3 folds, 5 folds, 10 folds, or 20 folds.

In some embodiments, the antigen binding proteins described herein can induce T cell activation. In some embodiments, the T cell activation level induced by the antigen binding proteins described herein is at least or about 1-fold, 2-fold, 3-fold, 4-fold, 5-fold, 6-fold, 7-fold, 8-fold, 9-fold, 10-fold, 20-fold, 50-fold, or 100-fold as compared to that induced by an isotype control antibody.

In some embodiments, the antigen binding protein as described herein does not induce immune response in normal cells (e.g., non-tumor cells) or in the absence of tumor cells.

In some embodiments, the antigen binding proteins as described herein are antagonists. In some embodiments, the antigen binding proteins are agonists.

In some embodiments, the antigen binding proteins can bind to a T cell. Thus, the antigen binding proteins can recruit T cells to a target cell. In some embodiments, the antigen binding proteins can bind to a tumor cell and an immune cell (e.g., T cell) in the same time, thereby bridging the interaction between cancer cell and the immune cell.

In some embodiments, the antigen binding protein specifically binds to an antigen as described herein with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹. In some embodiments, kinetic association rate (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rate (kon) is less than 1×10⁵/Ms, less than 1×10⁶/Ms, or less than 1×10⁷/Ms.

Affinities can be deduced from the quotient of the kinetic rate constants (Kd=koff/kon). In some embodiments, Kd is less than 1×10⁻⁴ M, less than 1×10⁻⁵ M, less than 1×10⁻⁶ M, less than 1×10⁻⁷ M, less than 1×10⁻⁸ M, less than 1×10⁻⁹ M, or less than 1×10⁻¹⁰ M. In some embodiments, the Kd is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, Kd is greater than 1×10′M, greater than 1×10⁻⁵ M, greater than 1×10⁻⁶ M, greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M.

General techniques for measuring the affinity of an antigen binding protein for an antigen include, e.g., ELISA, RIA, and surface plasmon resonance (SPR).

In some embodiments, the binding affinity of the antigen binding protein is compared against the binding affinity of the parental antibody (e.g., a monoclonal antibody or a nanobody where the antigen binding site in the antigen binding protein is derived from). In some embodiments, the binding affinity of the antigen binding site in the antigen binding protein is not reduced by more than 10%, 20%, 30% or 40%.

In some embodiments, ELISA can be used to measure the binding affinity. EC50 is calculated. The binding ratio of (1) EC50 of the antigen binding protein to (2) the EC50 of the parental antibody is not higher than 200%, 150%, 140%, 130%, 120%, 110%, or 100%. In fact, as the antigen binding protein may have more than one antigen binding sites to the same antigen, in some embodiments, the binding ratio is smaller than 0.9, 0.8, 0.7, 0.6, 0.5, or 0.4.

In some embodiments, thermal stabilities are determined. The antigen binding proteins as described herein can have a Tm greater than 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

As the antigen binding proteins can be described as a multi-domain protein, the melting curve sometimes shows two transitions, with a first denaturation temperature, Tm1, and a second denaturation temperature Tm2. The presence of these two peaks often indicates the denaturation of the two different domains (e.g., Fc, Fab, and/or VH-VL pairs). When there are two peaks, Tm1 refers to the first peak as the temperature increases. Thus, in some embodiments, the antigen binding proteins as described herein has a Tm1 greater than 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, the antigen binding proteins as described herein has a Tm2 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm, Tm1, Tm2 are less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the antigen binding protein has a high long-term stability. In some embodiments, the antigen binding protein is stable for at least about any one of 1 day, 3 days, 7 days, 2 weeks, 3 week, 4 weeks or more at about 4° C. In some embodiments, the antigen binding protein has a high long-term stability at an elevated temperature. In some embodiments, the antigen binding protein is stable for at least about any one of 1 day, 3 days, 7 days, 2 weeks, 3 week, 4 weeks or more at room temperature, e.g., at about 25° C. or higher. In some embodiments, the antigen binding protein is stable for at least about any one of 1 day, 2 days, 3 days, 4 days, 6 days, 7 days, 10 days, 2 weeks or more at physiological temperature, e.g., at about 37° C. or higher. In some embodiments, the antigen binding protein has a high long-term stability at a high concentration, e.g., at least or about 50 mg/mL, 100 mg/mL, 150 mg/mL, 200 mg/mL or higher.

A stable composition is substantially free (such as less than about any of 10%, 9%, 8%, 7%, 6%, 5%, 4%, 3%, 2%, 1% or less) of precipitation and/or aggregation. Precipitation can be detected by optical spectroscopy. Aggregation can be detected by e.g., DLS. In some embodiments, the antigen binding protein has high stability over freeze-thaw cycles. In some embodiments, a composition comprising the antigen binding protein can be freeze-thawed for at least about any one of 3, 4, 5, 6, 7, 8, 9, 10 times or more without losing structural integrity (e.g., forming aggregates) and/or activity of the antigen binding protein. In some embodiments, the composition comprising the antigen binding protein can be freeze-thawed at high concentration, e.g., at least or about 50 mg/mL, 100 mg/mL, 150 mg/mL, 200 mg/mL or higher.

In some embodiments, the antigen binding proteins or any antigen binding fragments can increase complement dependent cytotoxicity (CDC) by at least 10%, 20%, 30%, 40%, 50%, 60%, 70%, 80%, 90%, 100%, 2 folds, 3 folds, 5 folds, 10 folds, or 20 folds.

In some embodiments, the antigen binding proteins or any antigen binding fragments can increase antibody-dependent cell-mediated cytotoxicity (ADCC) by at least 10%, 20%, 30%, 40%, 50%, 60%, 70%, 80%, 90%, 100%, 2 folds, 3 folds, 5 folds, 10 folds, or 20 folds.

In some embodiments, the antigen binding proteins or any antigen binding fragments can increase phagocytosis rate by at least 10%, 20%, 30%, 40%, 50%, 60%, 70%, 80%, 90%, 100%, 2 folds, 3 folds, 5 folds, 10 folds, or 20 folds.

In some embodiments, the antigen binding proteins or any antigen binding fragments can enhance T cell function, for example, by increasing effector T cell proliferation and/or increasing gamma interferon production by the effector T cell (e.g., as compared to proliferation and/or cytokine production prior to treatment with the antigen binding proteins or antigen binding fragments).

In some embodiments, the antigen binding proteins or antigen binding fragments enhance CD4+ effector T cell function, for example, by increasing CD4+ effector T cell proliferation and/or increasing gamma interferon production by the CD4+ effector T cell (e.g., as compared to proliferation and/or cytokine production prior to treatment with the antigen binding proteins or antigen binding fragments). In some embodiments, the cytokine is gamma interferon. In some embodiments, the antigen binding proteins or antigen binding fragments increase number of intratumoral (infiltrating) CD4+ effector T cells (e.g., total number of CD4+ effector T cells, or e.g., percentage of CD4+ cells in CD45+ cells), e.g., as compared to number of intratumoral (infiltrating) CD4+ T cells prior to treatment with antigen binding proteins or antigen binding fragments. In some embodiments, the antigen binding proteins or antigen binding fragments increase number of intratumoral (infiltrating) CD4+ effector T cells that express gamma interferon (e.g., total gamma interferon expressing CD4+ cells, or e.g., percentage of gamma interferon expressing CD4+ cells in total CD4+ cells), e.g., as compared to number of intratumoral (infiltrating) CD4+ T cells that express gamma interferon prior to treatment.

In some embodiments, the antigen binding proteins increase number of intratumoral (infiltrating) CD8+ effector T cells (e.g., total number of CD8+ effector T cells, or e.g., percentage of CD8+ in CD45+ cells), e.g., as compared to number of intratumoral (infiltrating) CD8+T effector cells prior to treatment. In some embodiments, the antigen binding proteins increase number of intratumoral (infiltrating) CD8+ effector T cells that express gamma interferon (e.g., percentage of CD8+ cells that express gamma interferon in total CD8+ cells), e.g., compared to number of intratumoral (infiltrating) CD8+ T cells that express gamma interferon prior to treatment with the antigen binding protein.

In some embodiments, the antigen binding proteins enhance memory T cell function, for example by increasing memory T cell proliferation and/or increasing cytokine (e.g., gamma interferon) production by the memory cell.

In some embodiments, the antigen binding proteins have a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the Fc region is human IgG1, human IgG2, human IgG3, or human IgG4.

In some embodiments, the antigen binding proteins can induce apoptosis.

In some embodiments, the antigen binding proteins do not have a functional Fc region. For example, the antigen binding proteins are Fab, Fab′, F(ab′)2, and Fv fragments.

In some embodiments, the antigen binding proteins are humanized antibodies.

In some embodiments, the antigen binding proteins have a symmetric structure. In some embodiments, the antigen binding proteins have an asymmetric structure. In some embodiments, the multi-specific antigen binding protein described herein comprises 2, 3, 4, 5, or 6 antigen binding sites (e.g., antigen binding Fab domains, scFV, or nanobody (VHH)) that target a cancer antigen. In some embodiments, the multi-specific antigen binding protein described herein comprises 2, 3, 4, 5, or 6 antigen binding sites (e.g., antigen binding Fab domains, scFV, or nanobody (VHH)) that target a T cell specific antigen (e.g., CD3). In some embodiments, the multi-specific antigen binding protein described herein comprises at least 2, 3, 4, 5, 6, or 7 common light chains. In some embodiments, the at least 2, 3, 4, 5, 6, or 7 common light chains have the same VL sequence. In some embodiments, the C-terminus of a cancer-specific antigen binding Fab domain is connected (e.g., covalently connected or chemically connected) to the N-terminus of a neighboring cancer-specific antigen binding Fab domain within the same multi-specific antigen binding protein.

The present disclosure also provides an antigen binding protein, an antibody or antigen-binding fragment thereof that cross-competes with any antigen binding protein, antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of virology 70.3 (1996): 1863-1872, which is incorporated herein reference in its entirety. In one aspect, the present disclosure also provides an antigen binding protein, an antibody or antigen-binding fragment thereof that binds to the same epitope or region as any antigen binding protein, any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

### Antibodies and Antigen Binding Fragments

The antigen binding proteins as described herein can comprise various antibodies and antigen-binding fragments thereof as described herein. In some embodiments, one or more antigen binding sites (e.g., VHHs and/or VH/VL pairs) can be added to these antibodies or antigen-binding fragments thereof. In some embodiments, the antigen binding sites can be derived from these various antibodies and antigen-binding fragments as described herein.

In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and/or two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); Kontermann, R., & Dithel, S. (Eds.). (2010). Antibody engineering: Volume 2. Springer; each of which is incorporated herein by reference in its entirety. In some embodiments, the CDRs are based on Kabat definition. In some embodiments, the CDRs are based on the Chothia definition. In some embodiments, the CDRs are the longest CDR sequences as determined by Kabat, Chothia, AbM, IMGT, or contact definitions.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, rat, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the scFV has two heavy chain variable domains, and two light chain variable domains. In some embodiments, the scFV has two antigen binding regions, and the two antigen binding regions can bind to the respective target antigens with different affinities.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the antibodies or antigen-binding fragments thereof can bind to two different antigens or two different epitopes. In some embodiments, the antibodies or antigen-binding fragments thereof can bind to three different antigens or three different epitopes.

An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

In some embodiments, the scFv described herein comprises from N-terminus to C-terminus: VH; the polypeptide linker; and VL. In some embodiments, the scFv described herein comprises from N-terminus to C-terminus: VL; the polypeptide linker; and VH.

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

Linear antibodies comprise a pair of tandem Fd segments (VH-CH1-VH-CH1) which, together with complementary light chain polypeptides, form a pair of antigen binding regions. Linear antibodies can be bispecific or monospecific.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94: 7509-7514, 1997). Antibody homodimers can be converted to Fab′₂ homodimers through digestion with pepsin. Another way to form antibody homodimers is through the use of the autophilic T15 peptide described in Zhao et al. (J. Immunol. 25:396-404, 2002).

Antibodies and antibody fragments of the present disclosure can be modified in the Fc region to provide desired effector functions or serum half-life.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments (e.g., bispecific antibodies) described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of antigen binding proteins by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising a polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lad and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153: 516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antigen binding protein of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The protein complex (e.g., antigen binding proteins) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

The disclosure also provides a nucleic acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any nucleotide sequence as described herein, and an amino acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of sequence homology (e.g., amino acid sequence homology or nucleic acid homology) can also be determined. How to determine percentage of sequence homology is known in the art. In some embodiments, amino acid residues conserved with similar physicochemical properties (percent homology), e.g. leucine and isoleucine, can be used to measure sequence similarity. Families of amino acid residues having similar physicochemical properties have been defined in the art. These families include e.g., amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

### Methods of Making Antigen Binding Proteins

The antigen binding proteins as described herein can have antigen binding sites, or any portions derived from antibodies as described herein. An isolated protein for a fragment thereof can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide. The preparation can further include an adjuvant, such as Freund's complete or incomplete adjuvant, or a similar immunostimulatory agent.

Polyclonal antibodies can be prepared as described above by immunizing a suitable subject with a polypeptide, or an antigenic peptide thereof (e.g., part of the protein) as an immunogen. The antibody titer in the immunized subject can be monitored over time by standard techniques, such as with an enzyme-linked immunosorbent assay (ELISA) using the immobilized polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

VHH can also be obtained from naïve or designed synthetic llama VHH libraries, or from antibody engineering. PBMC from llamas can be obtained, and RNA can be isolated to generate cDNA by reverse transcription. Then, the VHH genes can be amplified by PCR and cloned to a phage display vector to construct the naïve VHH library. The synthetic (e.g., humanized) VHH library can be prepared by incorporation of shuffled VHH CDR1, 2 and 3, generated by overlapping PCR, to a modified human VH scaffold to generate enhanced diversity and keep low immunogenicity. The VHH libraries can be then panned against antigens to obtain VHH with desired binding affinities.

Various VHHs, VHs, and VLs can be used to make the multispecific antibodies as described herein. The sequences for VHHs, VHs, and VLs can be obtained e.g., from US 2017/0247475A1, US2019/0135907 A1, US20180327508A1, U.S. Ser. No. 10/093,742B2, US201213540, each of which is incorporated herein by reference in its entirety.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Phage display (panning) can be used to optimize antibody sequences with desired binding affinities. In this technique, a gene encoding single chain Fv (comprising VH or VL) can be inserted into a phage coat protein gene, causing the phage to “display” the scFv on its outside while containing the gene for the protein on its inside, resulting in a connection between genotype and phenotype. These displaying phages can then be screened against target antigens, in order to detect interaction between the displayed antigen binding sites and the target antigen. Thus, large libraries of proteins can be screened and amplified in a process called in vitro selection, and antibodies sequences with desired binding affinities can be obtained.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Identity or homology with respect to an original sequence is usually the percentage of amino acid residues present within the candidate sequence that are identical with a sequence present within the human, humanized, or chimeric antibody or fragment, after aligning the sequences and introducing gaps, if necessary, to achieve the maximum percent sequence identity, and not considering any conservative substitutions as part of the sequence identity.

In some embodiments, a covalent modification can be made to the antigen binding protein as described herein. These covalent modifications can be made by chemical or enzymatic synthesis, or by enzymatic or chemical cleavage. Other types of covalent modifications of the antigen binding protein are introduced into the molecule by reacting targeted amino acid residues of the antigen binding protein with an organic derivatization agent that is capable of reacting with selected side chains or the N- or C-terminal residues.

In some embodiments, the antigen binding protein is derived from an antibody variant. In some embodiments, antibody variants are provided having a carbohydrate structure that lacks fucose attached (directly or indirectly) to an Fc region. For example, the amount of fucose in such antigen binding protein may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region can be further engineered to replace the Asparagine at position 297 with Alanine (N297A).

In some embodiments, to facilitate production efficiency by avoiding Fab-arm exchange, the Fc region was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

In some embodiments, the multispecific antigen binding protein can be made by engineering the interface between a pair of heavy chain polypeptides (e.g., at constant domains) to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety. While the modification is made to Fc region, the present disclosure also shows that the modification is compatible with knobs-in-holes. The “knobs into holes” approach introduces a mutation for an amino acid with a large sidechain in one heavy chain, and a mutation for an amino acid with a small sidechain in the other heavy chain. Thus, the same heavy chains are less likely to associate with each other and the two different heavy chains have a higher chance to associate with each other. The “knobs into holes” approaches are described, e.g., in Ridgway, John B B, Leonard G. Presta, and Paul Carter. “Knobs-into-holes' engineering of antibody CH3 domains for heavy chain heterodimerization.” Protein Engineering, Design and Selection 9.7 (1996), which is incorporated herein by reference in its entirety.

In some embodiments, one or more amino acid residues in the CH3 portion of the IgG are substituted. In some embodiments, one heavy chain has one or more of the following substitutions Y349C and T366W. The other heavy chain can have one or more the following substitutions E356C, T366S, L368A, and Y407V. In some embodiments, one heavy chain has a T366Y, T366W, T366W/D399C, T366W/K392C, S354C/T366W, Y349C/T366W, E356C/T366W, Y349C/T366W, E357C/T366W, or Y349C/T366W (knob) substitution, and the other heavy chain has a Y407T, T366S/L368A/Y407V, T366S/L368A/K392C/Y407V, T366S/D399C/L368A/Y407V, Y349C/T366S/L368A/Y407V, S354C/T366S/L368A/Y407V, Y349C/T366S/L368A/Y407V, E356C/T366S/L368A/Y407V, Y349C/T366S/L368A/Y407V, or E357C/T366S/L368A/Y407V (hole) substitution (EU numbering). In some embodiments, one or more substitutions are selected from Y349C, T366W, T366S, T366Y, S354C, E356C, E357C, T366S, L368A, K392C, D399C, Y407V and Y407T. Some of the knobs into holes mutations are described, e.g., in U.S. Pat. No. 8,216,805B2, which is incorporated herein by reference.

Sequences encoding various antigen binding proteins can be constructed using various molecular biology techniques. In some embodiments, the sequences can be cloned into an expression vector (e.g., pcDNA3.3). Cells (e.g., Expi293) can be transfected with the constructed plasmids that can express the antigen binding proteins. The transfected cells can be then cultured and supernatant can be collected for protein purification.

In some embodiments, the antigen binding proteins can be purified by using Protein A chromatography. The obtained antigen binding proteins can be further analyzed by SDS-polyacrylamide gel electrophoresis (SDS-PAGE) and/or high performance liquid chromatography followed by size-exclusion chromatography (HPLC-SEC).

The present disclosure demonstrates that the antigen binding proteins as described herein can be expressed and purified easily. In some embodiments, antigen binding proteins are purified by Protein A chromatography. After purified by Protein A chromatography, the yield can be at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, or 500 mg/L. The purity of the antigen binding proteins can be at least or about 85%, 90%, 91%, 92%, 93%, 94%, 95%, 96%, 97%, 98%, or 99%, e.g., analyzed by HPLC-SEC.

### Methods of Treatment

The methods described herein include methods for the treatment of various disorders. Generally, the methods include administering a therapeutically effective amount of antigen binding proteins as described herein, to a subject who is in need of, or who has been determined to be in need of, such treatment. In some embodiments, the disorders are cancers, autoimmune diseases, infectious diseases, central nervous system diseases, metabolic diseases and the like.

As used in this context, to “treat” means to ameliorate at least one symptom of the disorder. In some embodiments, the disorder is cancer. Often, cancer results in death; thus, a treatment can result in an increased life expectancy (e.g., by at least 1, 2, 3, 4, 5, 6, 7, 8, 9, 10, 11, 12 months, or by at least 1, 2, 3, 4, 5, 6, 7, 8, 9, 10 years). Administration of a therapeutically effective amount of an agent described herein for the treatment of a condition associated with cancer will result in decreased number of cancer cells and/or alleviated symptoms.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In one aspect, the disclosure also provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antigen binding fragment disclosed herein to a subject in need thereof, e.g., a subject having, or identified or diagnosed as having, a cancer, e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer. In some embodiments, the cancer is melanoma, pancreatic carcinoma, mesothelioma, hematological malignancies, especially Non-Hodgkin's lymphoma, lymphoma, chronic lymphocytic leukemia, or advanced solid tumors.

In some embodiments, the compositions and methods disclosed herein can be used for treatment of patients at risk for a cancer. Patients with cancer can be identified with various methods known in the art.

In some embodiments, the compositions and methods disclosed herein can be used for treatment of patients at risk for an autoimmune disease.

In some embodiments, the compositions and methods disclosed herein can be used for treatment of infection, e.g., virus infection or bacterial infection.

As used herein, by an “effective amount” is meant an amount or dosage sufficient to effect beneficial or desired results including halting, slowing, retarding, or inhibiting progression of a disease. An effective amount will vary depending upon, e.g., an age and a body weight of a subject to which the antigen binding protein is to be administered, a severity of symptoms and a route of administration, and thus administration can be determined on an individual basis.

An effective amount can be administered in one or more administrations. By way of example, an effective amount of an antigen binding protein is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro.

Effective amounts and schedules for administering the antigen binding protein disclosed herein may be determined empirically, and making such determinations is within the skill in the art. Those skilled in the art will understand that the dosage that must be administered will vary depending on, for example, the mammal that will receive the antigen binding protein disclosed herein, the route of administration, the particular type of antigen binding protein, and/or compositions disclosed herein used and other drugs being administered to the mammal.

A typical daily dosage of an effective amount of an antigen binding protein is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antigen binding protein, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antigen binding proteins are administered in the same composition (e.g., a liquid composition). In some embodiments, at least one antigen binding protein, and at least one additional therapeutic agent are administered in the same composition (e.g., a liquid composition). In some embodiments, the at least one antigen binding protein and the at least one additional therapeutic agent are administered in two different compositions (e.g., a liquid composition containing at least one antigen binding protein and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antigen binding protein. In some embodiments, the one or more additional therapeutic agents and the at least one antigen binding protein are administered to the subject such that there is an overlap in the bioactive period of the one or more additional therapeutic agents and the at least one antigen binding protein in the subject.

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (ID01) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCLS, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, or an anti-GITR antibody.

### Pharmaceutical Compositions and Routes of Administration

Also provided herein are pharmaceutical compositions that contain at least one (e.g., one, two, three, or four) of the antigen binding proteins described herein. Two or more (e.g., two, three, or four) of any of the antigen binding proteins described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the therapeutic agent can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antigen binding proteins described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

The pharmaceutical compositions can be included in a container, pack, or dispenser together with instructions for administration. The disclosure also provides methods of manufacturing the antigen binding proteins, antibodies or antigen binding fragments thereof, or antibody-drug conjugates for various uses as described herein.

### EXAMPLES

The invention is further described in the following examples, which do not limit the scope of the invention described in the claims.

### Example 1: Materials and Methods

The materials used in the examples are listed in the following table.

TABLE 1

| Materials | Vendor | Cat. |
|---|---|---|
| Expi293 ™ Expression | Thermo Fisher | A14635 |
| System Kit | scientific | |
| Expi293 | Invitrogen | A14527 |
| ExpiFectamine293 Transfection Kit | Invitrogen | A14524 |
| Expi293 Expression Medium | Invitrogen | A1435101 |
| Protein A column | GE Healthcare | 175438 |
| SEC column | GE Healthcare | 28990944 |

### Generation of Multispecific Antibodies

Sequences encoding the multispecific antibodies were constructed according to the molecular biology protocols. The sequence for VHH targeting VEGF was obtained from VEGFBII0038 as described in US 2017/0247475A1. The sequence for VHH targeting Ang-2 was obtained from VHH 00938 as described in US2019/0135907 A1. The sequence for VHH targeting MSLN was obtained from MH6T as described in AU2018/265860 A1 or US20180327508A1. The sequence for VHH targeting GITR was obtained from hzC06 as described in U.S. Ser. No. 10/093,742B2. The sequence for VH and VL targeting PD-1 was obtained from MK-3475 as described in US2012135408. These sequences and other sequences in the foregoing applications are incorporated herein by reference in the entirety. The sequences were then cloned into a modified pcDNA3.3 expression vector. Expi293 cells were transfected with the constructed plasmids that can express the multispecific antibodies. The transfected cells were then cultured for 5 days and supernatant was collected for protein purification using a Protein A column (GE Healthcare, catalog number 175438). The obtained antibodies were analyzed by SDS-polyacrylamide gel electrophoresis (SDS-PAGE) and high performance liquid chromatography followed by size-exclusion chromatography (HPLC-SEC), then stored at −80° C.

### Purification of Multispecific Antibodies by HPLC-SEC

The supernatant of the antibody-expressing Expi293 cells (Thermo Fisher Scientific, catalog number A14635) was collected, and then filtered for protein purification using a Protein A column (GE Healthcare, catalog number 175438). The concentration of purified antibodies was measured according to the absorbance at 280 nm. The size and purity of the multispecific antibodies were tested by SDS-PAGE and HPLC-SEC, respectively. The purified antibodies were then stored at −80° C.

### Determination of Target Binding by ELISA

For enzyme-linked immunosorbent assay (ELISA), non-treated (by tissue culture) flat-bottom 96-well plates (Nunc MaxiSorp™, Thermo Fisher Scientific) were pre-coated with a proper concentration (0.25 mg/ml or 0.2 mg/ml) of the human target protein overnight at 4° C. After blocking with 2% bovine serum albumin (BSA), 100 μL 5-fold titrated antibodies from 100 nM to 0.00128 nM were pipetted into each well and incubated for 2 hours at ambient temperature. Following removal of the unbound substances, 100 μL 1:5000 diluted HRP-labeled goat anti-human IgG (Bethyl Laboratories, Inc., catalog number A80-304P) were added to the wells and incubated for 1 hour. The color in each well was developed by dispensing 100 μL TMB substrate, and was then stopped by adding 100 μL 2 M HCl. The absorbance values at 450 nm and 540 nm were measured using a microplate spectrophotometer (SpectraMax® M5e).

### Determination of Thermal Stability by Differential Scanning Fluorimetry (DSF)

Melting temperature (Tm) of antibodies was determined using QuantStudio™ 7 Flex Real-Time PCR system (Applied Biosystems). Specifically, 19 μL of the antibody solution was mixed with 1 μL of 80× SYPRO Orange solution (Invitrogen), and the mixture was then transferred to a 96-well plate (Biosystems). The plate was heated from 26° C. to 95° C. at a rate of 0.9° C./min, and the resulting fluorescence data were collected. The negative derivatives of the fluorescence changes with respect to different temperatures were calculated, and the maximal value was defined as the melting temperature Tₘ. Alternatively, if a protein exhibited multiple unfolding transitions, the first two Tₘ values were calculated as Tₘ1 and Tₘ2. Data collection and Tₘ calculation were conducted automatically by the operation software (QuantStudio™ Real Time PCR software v1.3).

### Example 2: Multispecific Antibodies

Seven multispecific antibodies targeting VEGF and Ang-2 were designed with schematic structures as shown in FIGS. 1B-1H. T1 and U1 represent VHHs targeting VEGF and Ang-2 respectively. The multispecific antibodies were named as W366001-T1U1.F82-1.uIgG4V1 (or “F82”), W366001-T1U1.F83-1.uIgG4V1 (or “F83”), W366001-U1T1.F84-1.uIgG4V1 (or “F84”), W366001-U1T1.G1-1.uIgG4V1 (or “G1”), W366001-U1T1.G32-1.uIgG4V1 (or “G32”), W366001-U1T1.G33-1.uIgG4V1 (or “G33”), and W366001-U1T1.H9-1.uIgG4V1 (or “H9”), respectively.

The multispecific antibodies were purified by a protein A column. If the purity of samples was less than 90%, the samples were then further purified by the HPLC-SEC purification. The yield and purity after the purification were determined. The melting temperature (e.g., Tₘ1 and/or Tₘ2) was determined by DSF. ELISA was also performed to determine the binding affinity to the target. EC50 was calculated and was compared against the parental antibody. If the tested multispecific antibody is single valent for a target, a single valent parental antibody with the same binding site was selected for comparison purpose. If the tested multispecific antibody is multivalent (e.g., bivalent) for a target, a bivalent parental antibody with the same binding sites were selected for comparison purpose. The binding ratio (ratio of EC50) of each multispecific antibody relative to that of the parental antibody was also determined. The results are summarized in the table below.

TABLE 2

| | | | | | Binding Ratio | |
|---|---|---|---|---|---|---|
| | | Final | Final | DSF | (Multispecific/ | |
| | Protein | Yield | Purity | Tₘ1 | Parental mAb) | |
| No. | Name | (mg/L) | SEC (%) | (° C.) | VEGF | Ang-2 |
| 1 | F82 | 146.5 | 95% | 61.6 | 1.3 | 0.6 |
| 2 | F83 | 227.5 | 93% | 61.8 | 1.1 | 0.9 |
| 3 | F84 | 211.1 | 95% | 61.1 | 2.0 | 0.6 |
| 4 | G1 | 209.9 | 98% | 61.8 | 0.4 | 0.6 |
| 5 | G32 | 192.6 | 98% | 58.5 | 0.4 | 0.5 |
| 6 | G33 | 137.7 | 93% | 61.1 | 0.4 | 0.5 |
| 7 | H9 | 107.8 | 99% | 58.5 | 0.4 | 0.4 |

### a) F82

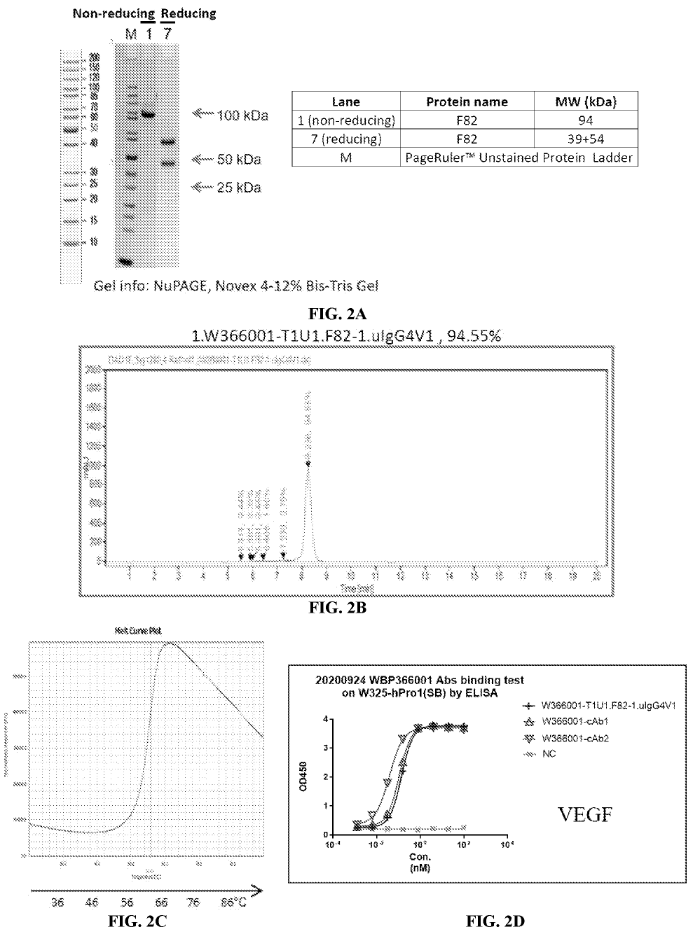

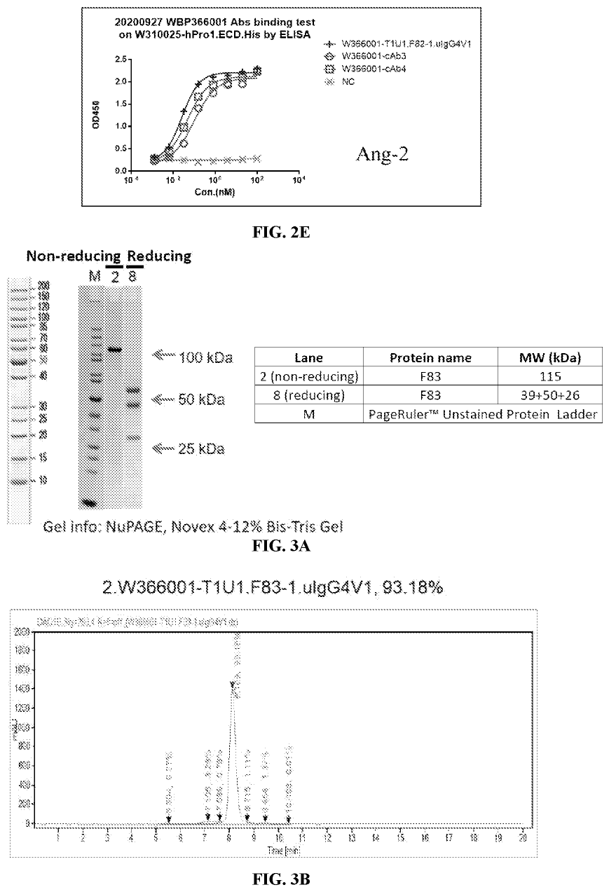

The schematic structure of the multispecific antibody F82 is shown in FIG. 1B. After purification, the purified F82 was analyzed by non-reducing and reducing gel electrophoresis, respectively. As shown in FIG. 2A, the results indicate that the F82 antibody was successfully purified with correct molecular weight. The SEC analysis result is shown in FIG. 2B, in which a single major peak was observed. The final purity of the F82 antibody was determined as 94.55%. In addition, the Tₘ1 value of F82 was determined as 61.6° C., based on the melting curve shown in FIG. 2C.

In addition, the binding capabilities of F82 to VEGF and Ang-2 were measured by ELISA, and the results are shown in FIG. 2D and FIG. 2E, respectively. The EC50 values of F82 binding to VEGF and Ang-2 were determined as 0.139 μg/ml and 0.026 μg/ml, respectively.

### b) F83

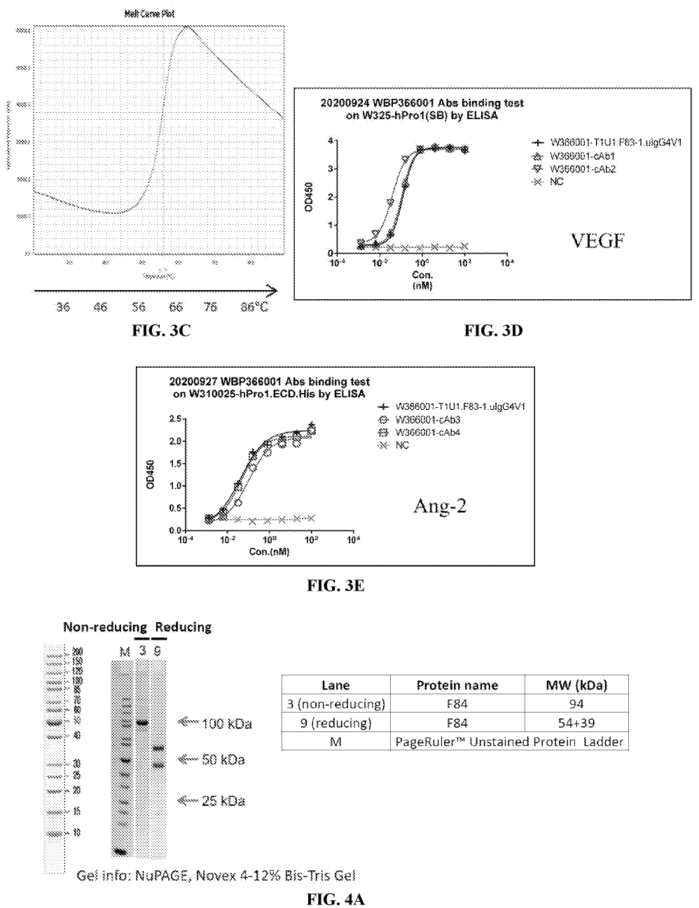

The schematic structure of the multispecific antibody F83 is shown in FIG. 1C. After purification, the purified F83 was analyzed by non-reducing and reducing gel electrophoresis, respectively. As shown in FIG. 3A, the results indicate that the F83 antibody was successfully purified with correct molecular weight. The SEC analysis result is shown in FIG. 3B, in which a single major peak was observed. The final purity of the F83 antibody was determined as 93.18%. In addition, the Tₘ1 and Tₘ2 values of F83 was determined as 61.8° C. and 67.2° C., respectively, based on the melting curve shown in FIG. 3C.

The binding capabilities of F83 to VEGF and Ang-2 were measured by ELISA, and the results are shown in FIG. 3D and FIG. 3E, respectively. The EC50 values of F83 binding to VEGF and Ang-2 were determined as 0.122 μg/ml and 0.041 μg/ml, respectively.

### c) F84

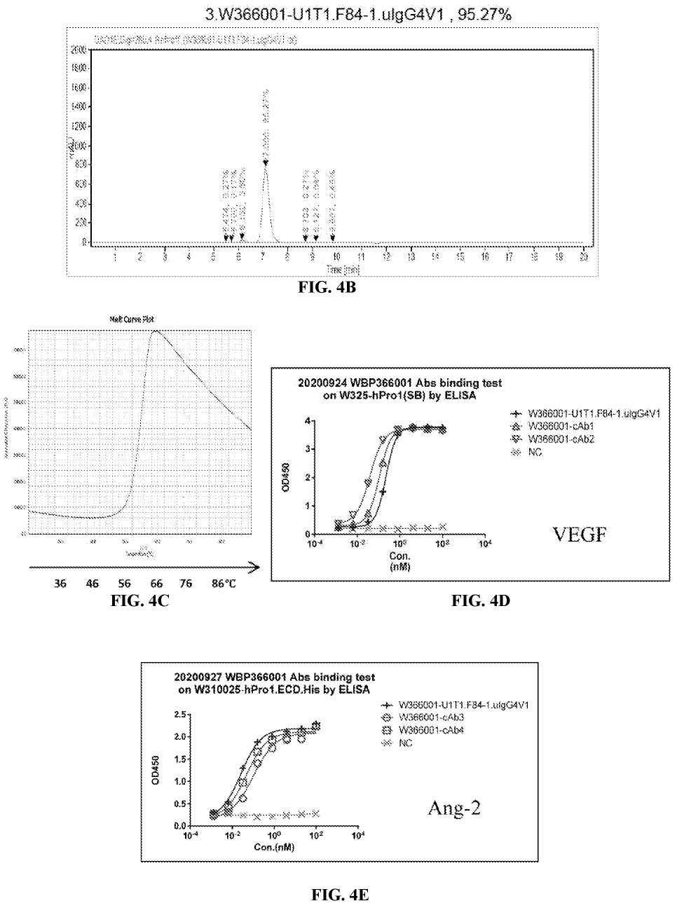

The schematic structure of the multispecific antibody F84 is shown in FIG. 1D. After purification, the purified F84 was analyzed by non-reducing and reducing gel electrophoresis, respectively. As shown in FIG. 4A, the results indicate that the F84 antibody was successfully purified with correct molecular weight. The SEC analysis result is shown in FIG. 4B, in which a single major peak was observed. The final purity of the F84 antibody was determined as 95.27%. In addition, the Tₘ1 value of F84 was determined as 61.1° C., based on the melting curve shown in FIG. 4C.

The binding capabilities of F84 to VEGF and Ang-2 were measured by ELISA, and the results are shown in FIG. 4D and FIG. 4E, respectively. The EC50 values of F84 binding to VEGF and Ang-2 were determined as 0.222 μg/ml and 0.026 μg/ml, respectively.

### d) G1

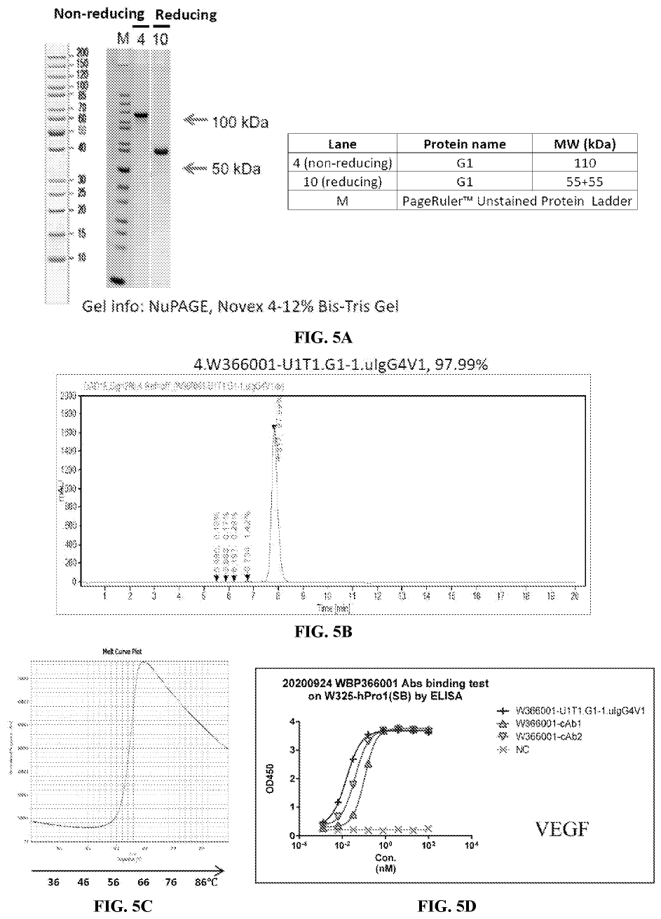

The schematic structure of the multispecific antibody G1 is shown in FIG. 1E. After purification, the purified G1 was analyzed by non-reducing and reducing gel electrophoresis, respectively. As shown in FIG. 5A, the results indicate that the G1 antibody was successfully purified with correct molecular weight. The SEC analysis result is shown in FIG. 5B, in which a single major peak was observed. The final purity of the G1 antibody was determined as 97.99%. In addition, the Tₘ1 value of G1 was determined as 61.8° C., based on the melting curve shown in FIG. 5C.

The binding capabilities of G1 to VEGF and Ang-2 were measured by ELISA, and the results are shown in FIG. 5D and FIG. 5E, respectively. The EC50 values of G1 binding to VEGF and Ang-2 were determined as 0.016 μg/ml and 0.030 μg/ml, respectively.

### e) G32

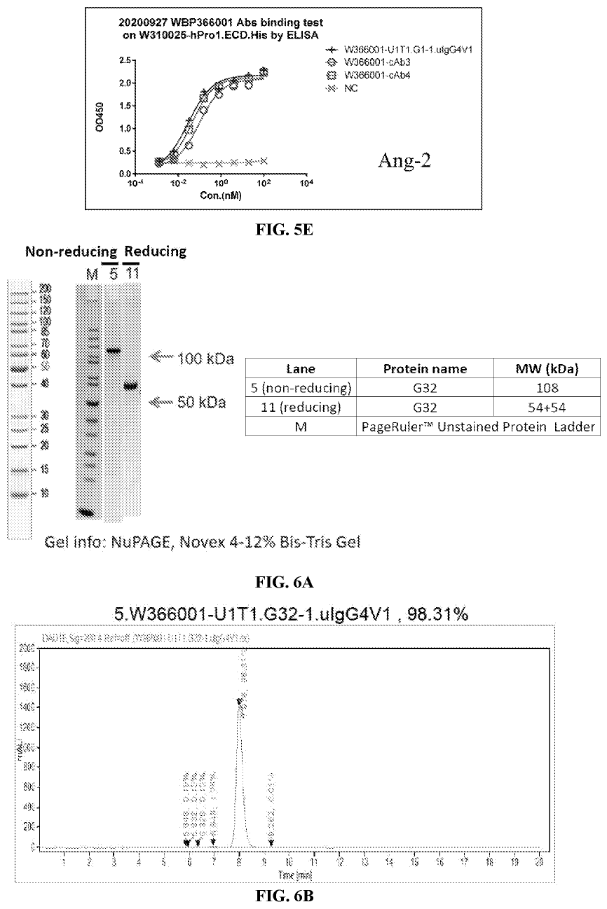

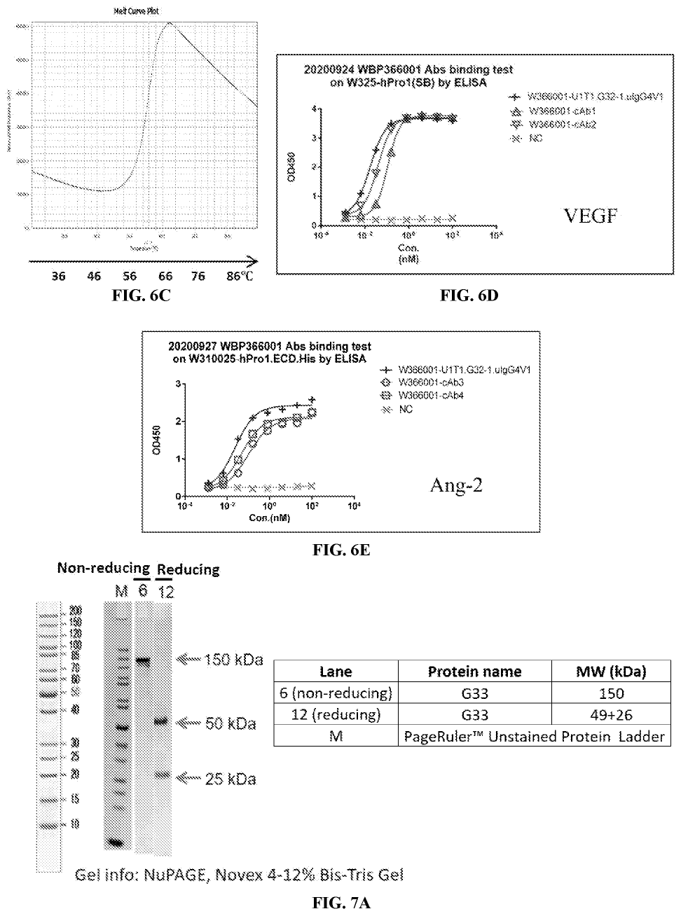

The schematic structure of the multispecific antibody G32 is shown in FIG. 1F. After purification, the purified G32 was analyzed by non-reducing and reducing gel electrophoresis, respectively. As shown in FIG. 6A, the results indicate that the G32 antibody was successfully purified with correct molecular weight. The analysis SEC result is shown in FIG. 6B, in which a single major peak was observed. The final purity of the G32 antibody was determined as 98.31%. In addition, the Tₘ1 value of G32 was determined as 58.5° C., based on the melting curve shown in FIG. 6C.

The binding capabilities of G32 to VEGF and Ang-2 were measured by ELISA, and the results are shown in FIG. 6D and FIG. 6E, respectively. The EC50 values of G32 binding to VEGF and Ang-2 were determined as 0.017 μg/ml and 0.023 μg/ml, respectively.

### f) G33

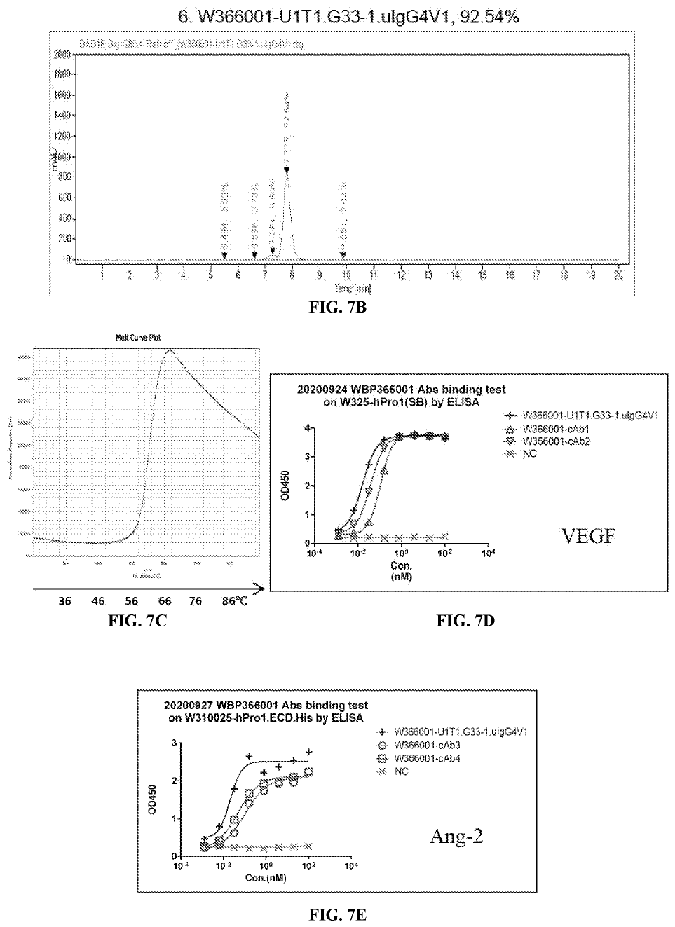

The schematic structure of the multispecific antibody G33 is shown in FIG. 1G. After purification, the purified G33 was analyzed by non-reducing and reducing gel electrophoresis, respectively. As shown in FIG. 7A, the results indicate that the G33 antibody was successfully purified with correct molecular weight. The SEC analysis result is shown in FIG. 7B, in which a single major peak was observed. The final purity of the G33 antibody was determined as 92.54%. In addition, the Tₘ1 value of G33 was determined as 61.1° C., based on the melting curve shown in FIG. 7C.

The binding capabilities of G33 to VEGF and Ang-2 were measured by ELISA, and the results are shown in FIG. 7D and FIG. 7E, respectively. The EC50 values of G33 binding to VEGF and Ang-2 were determined as 0.016 μg/ml and 0.021 μg/ml, respectively.

### g) H9

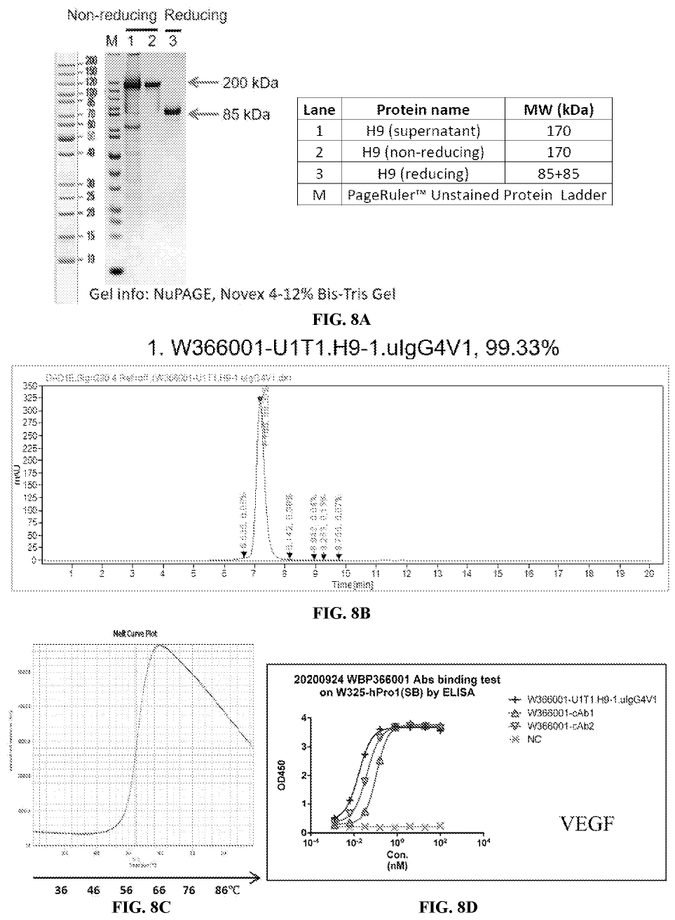

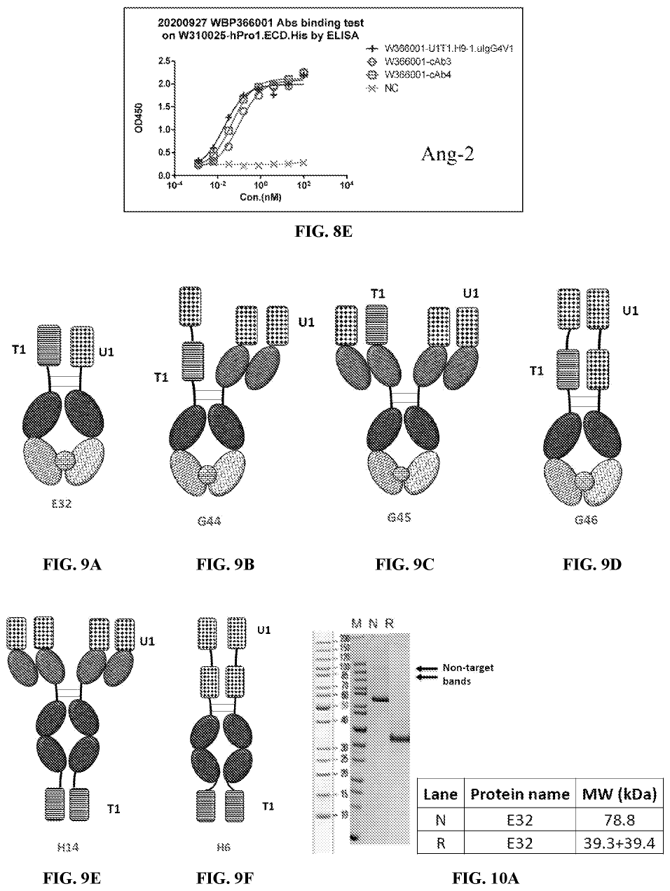

The schematic structure of the multispecific antibody H9 is shown in FIG. 1H. After purification, the purified H9 was analyzed by non-reducing and reducing gel electrophoresis, respectively. As shown in FIG. 8A, the results indicate that the H9 antibody was successfully purified with correct molecular weight. The SEC analysis result is shown in FIG. 8B, in which a single major peak was observed. The final purity of the H9 antibody was determined as 99.33%. In addition, the Tₘ1 value of H9 was determined as 58.5° C., based on the melting curve shown in FIG. 8C.

The binding capabilities of H9 to VEGF and Ang-2 were measured by ELISA, and the results are shown in FIG. 8D and FIG. 8E, respectively. The EC50 values of H9 binding to VEGF and Ang-2 were determined as 0.017 μg/ml and 0.020 μg/ml, respectively.

### Example 3: Multispecific Antibodies Targeting VEGF and Ang-2

Six multispecific antibodies targeting VEGF and Ang-2 were designed with schematic structures as shown in FIGS. 9A-9F. T1 and U1 represent VHHs targeting VEGF and Ang-2, respectively. The multispecific antibodies were named as W366001-T1U1.E32-1.uIgG4V1 (or “E32”), W366001-U1T1.G44-1.uIgG4V1 (or “G44”), W366001-U1T1.G45-1.uIgG4V1 (or “G45”), W366001-U1T1.G46-1.uIgG4V1 (or “G46”), W366001-U1T1.H14-1.uIgG4V1 (or “H14”), and W366001-U1T1.H6-1.uIgG4V1 (or “H6”), respectively.

The multispecific antibodies were purified by a protein A column. If the purity of samples was less than 90%, the samples were then further purified by the HPLC-SEC purification. The yield and purity after the purification were determined. The melting temperature (e.g., Tₘ1 and/or Tₘ2) was determined by DSF. ELISA was also performed to determine the binding affinity to the target. EC50 was calculated and was compared against the parental antibody. If the tested multispecific antibody is single valent for a target, a single valent parental antibody with the same binding site was selected for comparison purpose. If the tested multispecific antibody is multivalent (e.g., bivalent) for a target, a bivalent parental antibody with the same binding sites were selected for comparison purpose. The binding ratio (ratio of EC50) of each multispecific antibody relative to that of the parental antibody was also determined. The results are summarized in the table below.

TABLE 3

| | | | | | Binding Ratio | |
|---|---|---|---|---|---|---|
| | | Final | Final | DSF | (Multispecific/ | |
| | Protein | Yield | Purity | Tₘ1 | Parental mAb) | |
| No. | Name | (mg/L) | SEC (%) | (° C.) | VEGF | Ang-2 |
| 1 | E32 | 246.3 | 92.88% | 63.3 | 1.477 | 0.646 |
| 2 | G44 | 149.9 | 91.31% | 62.5 | 5.230 | 0.354 |
| 3 | G45 | 32.4 | 99.07% | 62.6 | 2.897 | N/A |
| 4 | G46 | 150.9 | 90.51% | 63.1 | 7.385 | 0.475 |
| 5 | H14 | 79.3 | 90.32% | 63.1 | 0.513 | 0.468 |
| 6 | H6 | 100.1 | 95.80% | 61.5 | 0.513 | 0.319 |

### a) E32

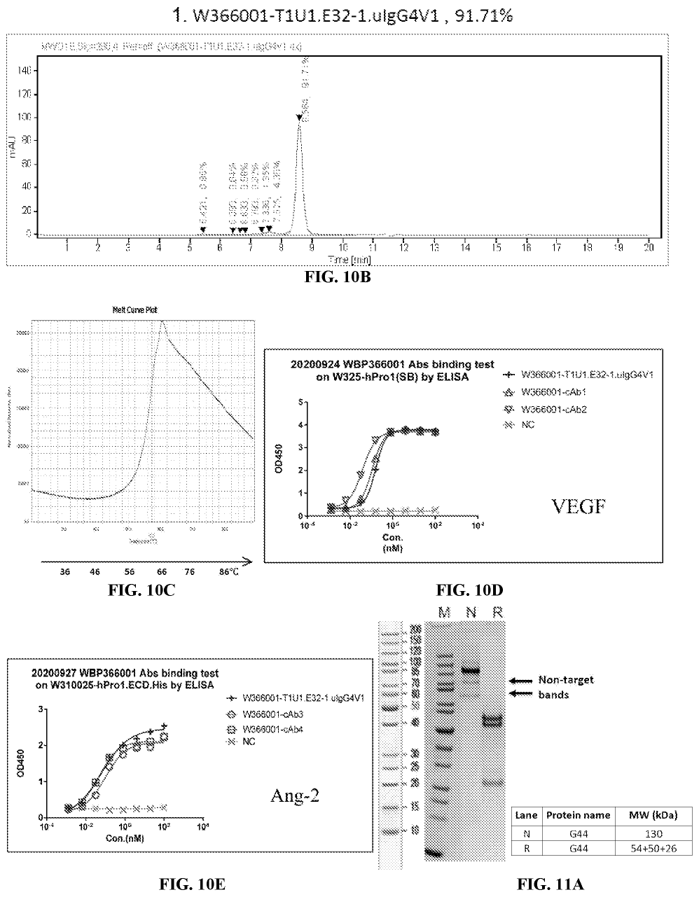

The schematic structure of the multispecific antibody E32 is shown in FIG. 9A. After purification, the purified E32 was analyzed by non-reducing (N) and reducing (R) gel electrophoresis, respectively. As shown in FIG. 10A, multiple non-target bands were observed in the purified E32 antibody sample. The SEC analysis result is shown in FIG. 10B, in which a single major peak was observed. The final purity of the E32 antibody was determined as 91.71%. In addition, the Tₘ1 value of E32 was determined as 63.3° C., based on the melting curve shown in FIG. 10C.

The binding capabilities of E32 to VEGF and Ang-2 were measured by ELISA, and the results are shown in FIG. 10D and FIG. 10E, respectively. The EC50 values of E32 binding to VEGF and Ang-2 were determined as 0.161 μg/ml and 0.064 μg/ml, respectively.

### b) G44

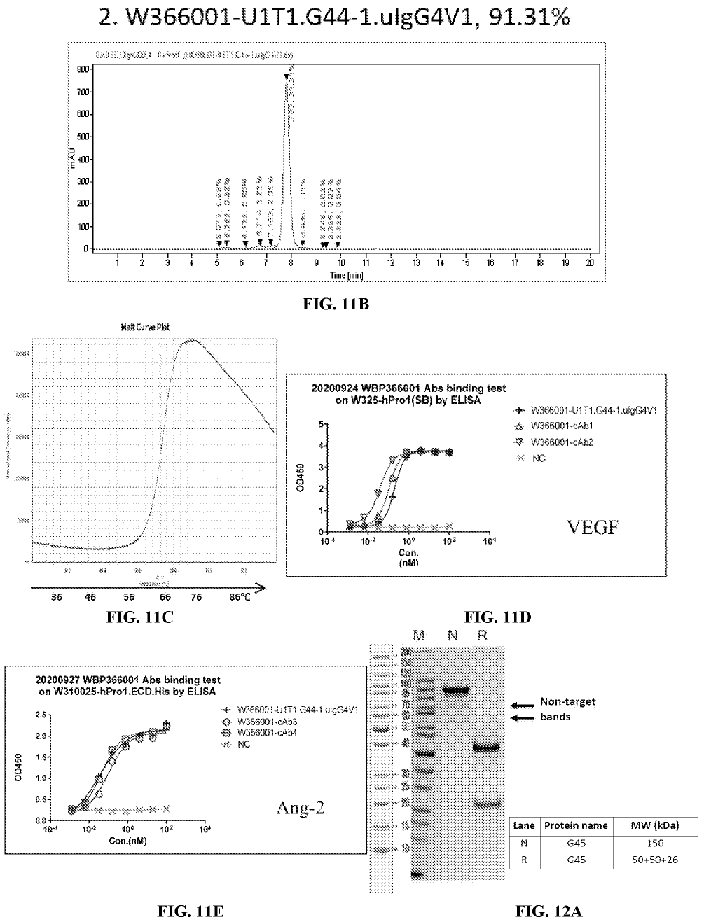

The schematic structure of the multispecific antibody G44 is shown in FIG. 9B. After purification, the purified G44 was analyzed by non-reducing (N) and reducing (R) gel electrophoresis, respectively. As shown in FIG. 11A, multiple non-target bands were observed in the purified G44 antibody sample. The SEC analysis result is shown in FIG. 11B, in which a single major peak was observed. The final purity of the G44 antibody was determined as 91.31%. In addition, the Tₘ1 value of G44 was determined as 62.5° C., based on the melting curve shown in FIG. 11C.

The binding capabilities of G44 to VEGF and Ang-2 were measured by ELISA, and the results are shown in FIG. 11D and FIG. 11E, respectively. The EC50 values of G44 binding to VEGF and Ang-2 were determined as 0.204 μg/ml and 0.055 μg/ml, respectively.

### c) G45

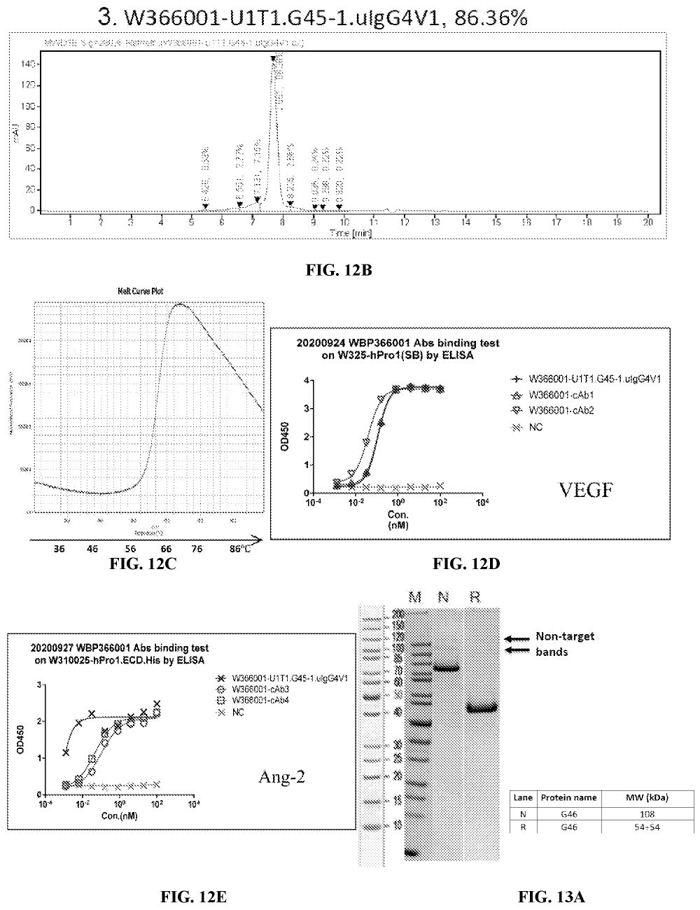

The schematic structure of the multispecific antibody G45 is shown in FIG. 9C. After purification, the purified G45 was analyzed by non-reducing (N) and reducing (R) gel electrophoresis, respectively. As shown in FIG. 12A, multiple non-target bands were observed in the purified G45 antibody sample. The SEC analysis result is shown in FIG. 12B, in which a single major peak was observed. The final purity of the G45 antibody was determined as 86.36%. In addition, the Tₘ1 value of G45 was determined as 62.6° C., based on the melting curve shown in FIG. 12C.

The binding capabilities of G45 to VEGF and Ang-2 were measured by ELISA, and the results are shown in FIG. 12D and FIG. 12E, respectively. The EC50 values of G45 binding to VEGF was determined as 0.113 μg/ml.

### d) G46

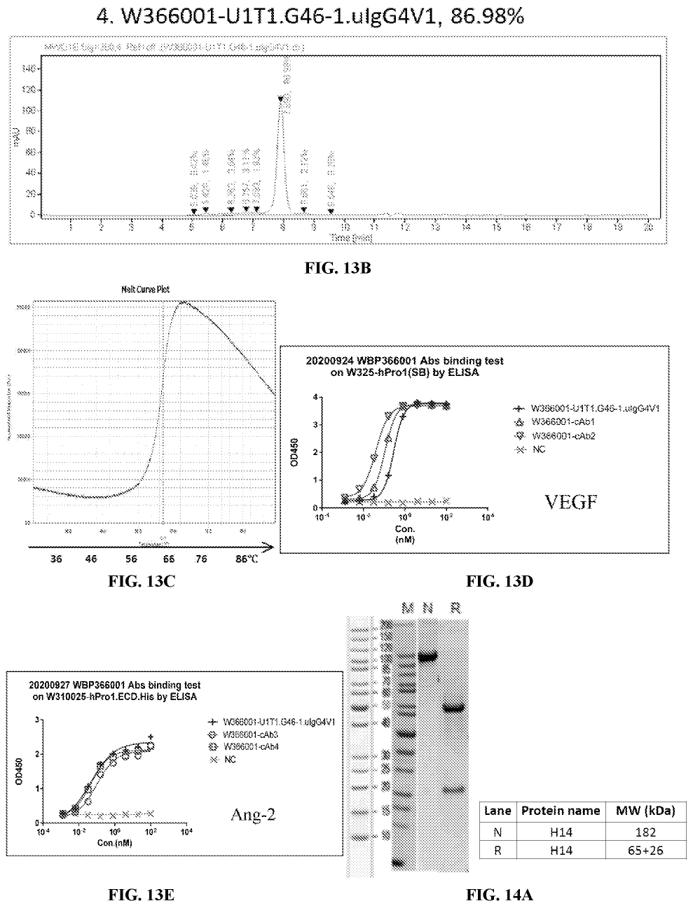

The schematic structure of the multispecific antibody G46 is shown in FIG. 9D. After purification, the purified G46 was analyzed by non-reducing (N) and reducing (R) gel electrophoresis, respectively. As shown in FIG. 13A, multiple non-target bands were observed in the purified G46 antibody sample. The SEC analysis result is shown in FIG. 13B, in which a single major peak was observed. The final purity of the G46 antibody was determined as 86.98%. In addition, the Tₘ1 value of G46 was determined as 63.1° C., based on the melting curve shown in FIG. 13C.

The binding capabilities of G46 to VEGF and Ang-2 were measured by ELISA, and the results are shown in FIG. 13D and FIG. 13E, respectively. The EC50 values of G46 binding to VEGF and Ang-2 were determined as 0.288 μg/ml and 0.047 μg/ml, respectively.

### e) H14

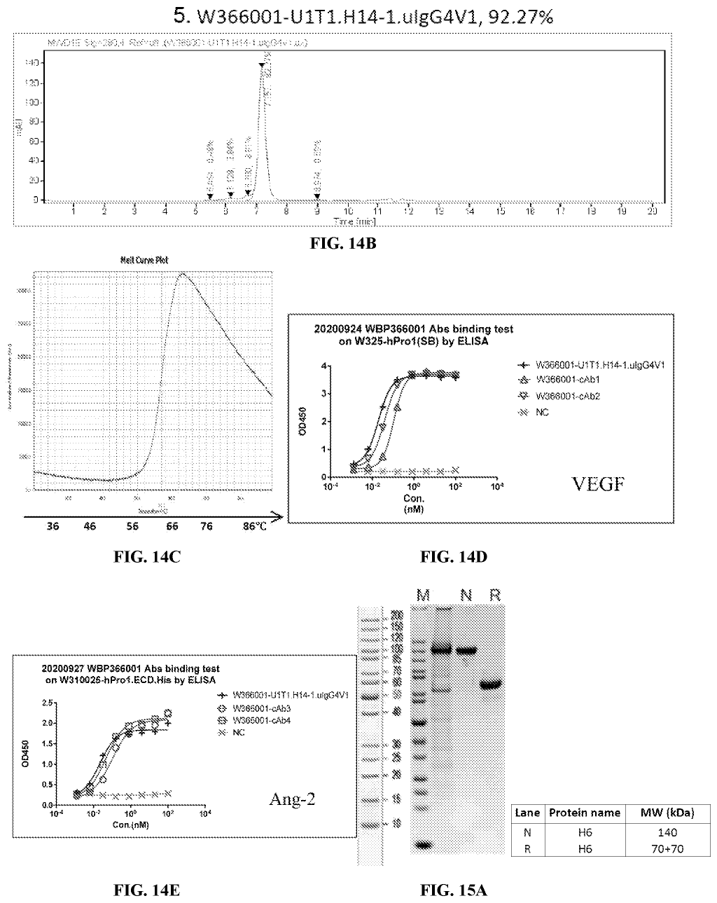

The schematic structure of the multispecific antibody H14 is shown in FIG. 9E. After purification, the purified H14 was analyzed by non-reducing (N) and reducing (R) gel electrophoresis, respectively. As shown in FIG. 14A, the results indicate that the H14 antibody was successfully purified with correct molecular weight. The SEC analysis result is shown in FIG. 14B, in which a single major peak was observed. The final purity of the H14 antibody was determined as 92.27%. In addition, the Tₘ1 value of H14 was determined as 63.1° C., based on the melting curve shown in FIG. 14C.

The binding capabilities of H14 to VEGF and Ang-2 were measured by ELISA, and the results are shown in FIG. 14D and FIG. 14E, respectively. The EC50 values of H14 binding to VEGF and Ang-2 were determined as 0.020 μg/ml and 0.022 μg/ml, respectively.

### f) H6

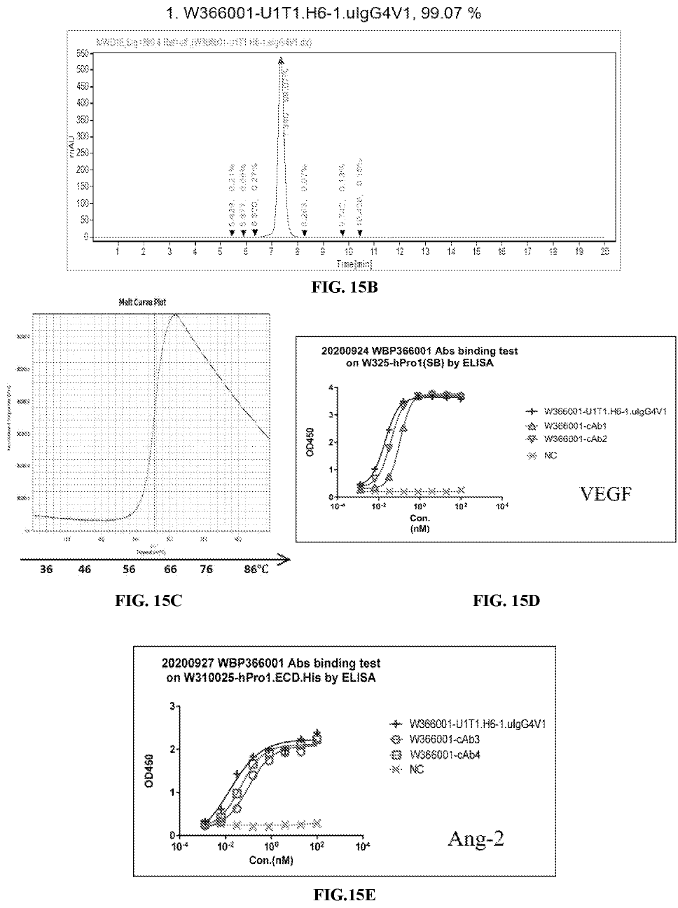

The schematic structure of the multispecific antibody H6 is shown in FIG. 9F. After purification, the purified H6 was analyzed by non-reducing (N) and reducing (R) gel electrophoresis, respectively. As shown in FIG. 15A, the results indicate that the H6 antibody was successfully purified with correct molecular weight. The SEC analysis result is shown in FIG. 15B, in which a single major peak was observed. The final purity of the H6 antibody was determined as 99.07%. In addition, the Tₘ1 value of H6 was determined as 61.5° C., based on the melting curve shown in FIG. 15C.

The binding capabilities of H6 to VEGF and Ang-2 were measured by ELISA, and the results are shown in FIG. 15D and FIG. 15E, respectively. The EC50 values of H6 binding to VEGF and Ang-2 were determined as 0.020 μg/ml and 0.015 μg/ml, respectively.

### Example 4: Multispecific Antibodies Targeting VEGF and PD-1

Eight multispecific antibodies targeting VEGF and PD-1 were designed with schematic structures as shown in FIGS. 16A-16H. T1 represents a VHH targeting VEGF. U12 represents a Fab domain targeting PD-1. The multispecific antibodies were named as W366002-U12T1.E28-1.uIgG4V1 (or “E28”), W366002-T1U12.F43-1.uIgG4V1 (or “F43”), W366002-U12T1.F85R-1.uIgG4V1 (or F85R), W366002-U12T1.F45R-1.uIgG4V1 (or “F45R”), W366002-U12T1.G58-1.uIgG4V1 (or “G58”), W366002-T1U12.H27-1.uIgG4V1 (or “H27”), W366002-T1U12.H22-1.uIgG4V1 (or “H22”), and W366002-T1U12.G47-1.uIgG4V1 (or “G47”), respectively.

The multispecific antibodies were purified by a protein A column. If the purity of samples was less than 90%, the samples were then further purified by the HPLC-SEC purification. The yield and purity after the purification were determined. The melting temperature (e.g., Tₘ1 and/or Tₘ2) was determined by DSF. ELISA was also performed to determine the binding affinity to the target. EC50 was calculated and was compared against the parental antibody. If the tested multispecific antibody is single valent for a target, a single valent parental antibody with the same binding site was selected for comparison purpose. If the tested multispecific antibody is multivalent (e.g., bivalent) for a target, a bivalent parental antibody with the same binding sites were selected for comparison purpose. The binding ratio (ratio of EC50) of each multispecific antibody relative to that of the parental antibody was also determined. The results are summarized in the table below.

TABLE 4

| | | | | | | Binding Ratio | |
|---|---|---|---|---|---|---|---|
| | | Final | Final | Purifica- | DSF | (Multispecific/ | |
| | Protein | Yield | Purity | tion | Tₘ1 | Parental mAb) | |
| No. | Name | (mg/L) | SEC (%) | Steps | (° C.) | VEGF | PD-1 |
| 1 | E28 | 90.57 | 97.85% | 1 | 63.4 | 0.51 | 1.14 |
| 2 | F43 | 68.36 | 91.88% | 1 | 64.7 | 0.93 | 1.33 |
| 3 | F85R | 105.16 | 96.68% | 1 | 61.5 | 1.02 | 0.92 |
| 4 | F45R | 97.93 | 92.75% | 1 | 64.6 | 1.07 | 0.55 |
| 5 | G58 | 21.66 | 91.71% | 2 | 62.0 | 0.56 | 1.07 |
| 6 | H27 | 10.66 | 90.25% | 2 | 62.9 | 0.79 | 1.15 |
| 7 | H22 | 23.97 | 66.73% + | 2 | 63.1 | 0.53 | 0.74 |
| | | | 24.08% | | | | |
| 8 | G47 | 85.96 | 94.68% | 1 | 62.8 | 1.02 | 0.48 |

### a) E28

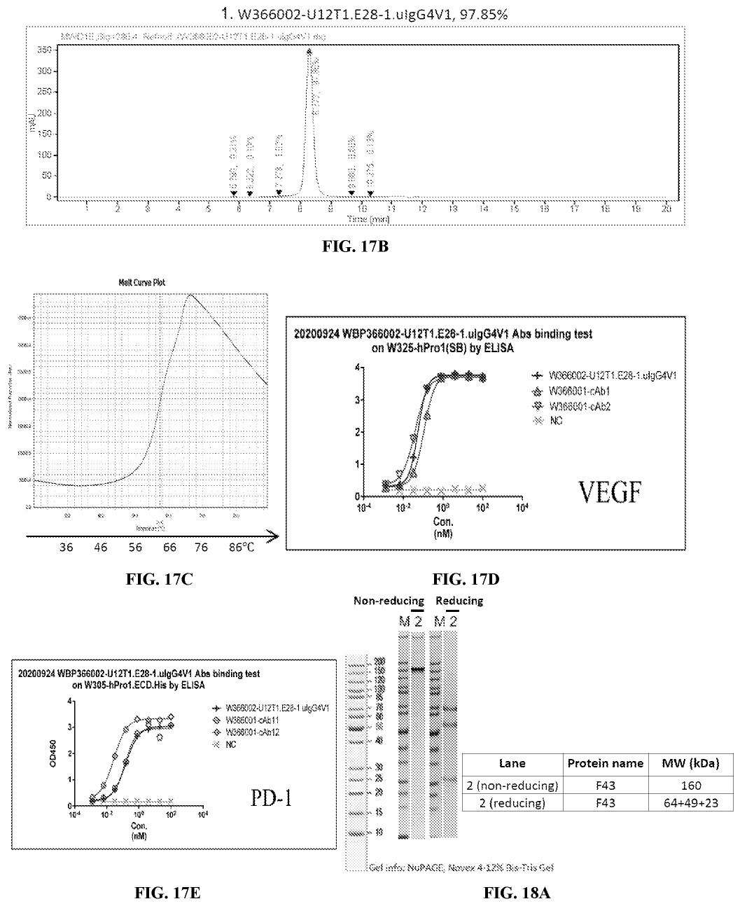

The schematic structure of the multispecific antibody E28 is shown in FIG. 16A. After purification, the purified E28 was analyzed by non-reducing and reducing gel electrophoresis, respectively. As shown in FIG. 17A, the results indicate that the E28 antibody was successfully purified with correct molecular weight. The SEC result is shown in FIG. 17B, in which a single major peak was observed. The final purity of the E28 antibody was determined as 97.85%. In addition, the Tₘ1 and Tₘ2 values of E28 were determined as 63.4° C. and 70.0° C., respectively, based on the melting curve shown in FIG. 17C.

In addition, the binding capabilities of E28 to VEGF and PD-1 were measured by ELISA, and the results are shown in FIG. 17D and FIG. 17E, respectively. The EC50 values of E28 binding to VEGF and PD-1 were determined as 0.056 μg/ml and 0.154 μg/ml, respectively.

### b) F43

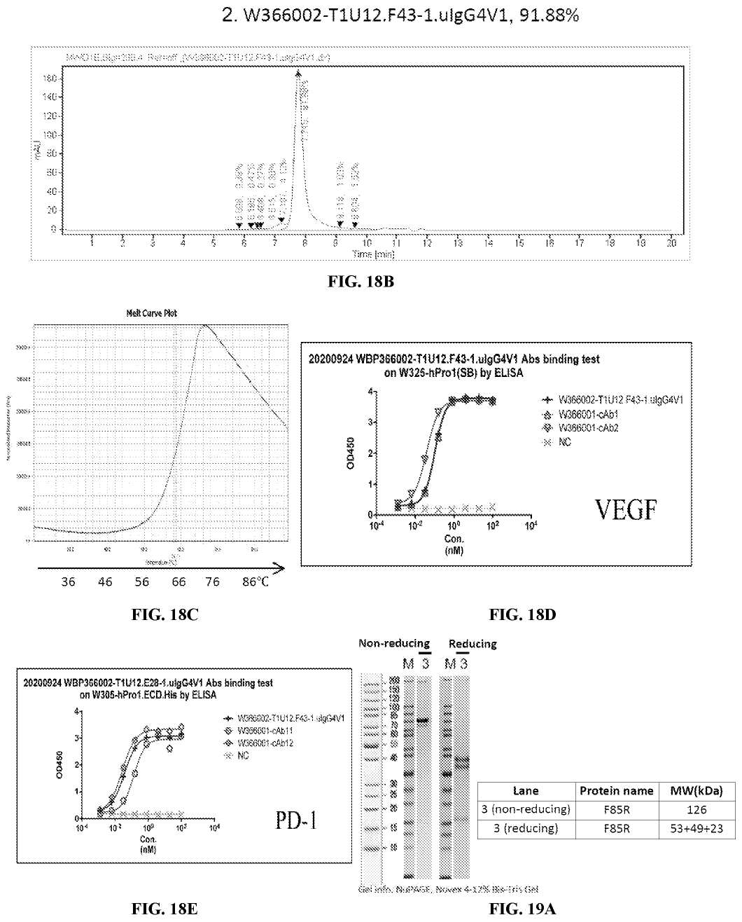

The schematic structure of the multispecific antibody F43 is shown in FIG. 16B. After purification, the purified F43 was analyzed by non-reducing and reducing gel electrophoresis, respectively. As shown in FIG. 18A, the results indicate that the F43 antibody was successfully purified with correct molecular weight. The SEC result is shown in FIG. 18B, in which a single major peak was observed. The final purity of the F43 antibody was determined as 91.88%. In addition, the Tₘ1 and Tₘ2 values of F43 were determined as 64.7° C. and 69.7° C., respectively, based on the melting curve shown in FIG. 18C.

In addition, the binding capabilities of F43 to VEGF and PD-1 were measured by ELISA, and the results are shown in FIG. 18D and FIG. 18E, respectively. The EC50 values of F43 binding to VEGF and PD-1 were determined as 0.102 μg/ml and 0.306 μg/ml, respectively.

### c) F85R

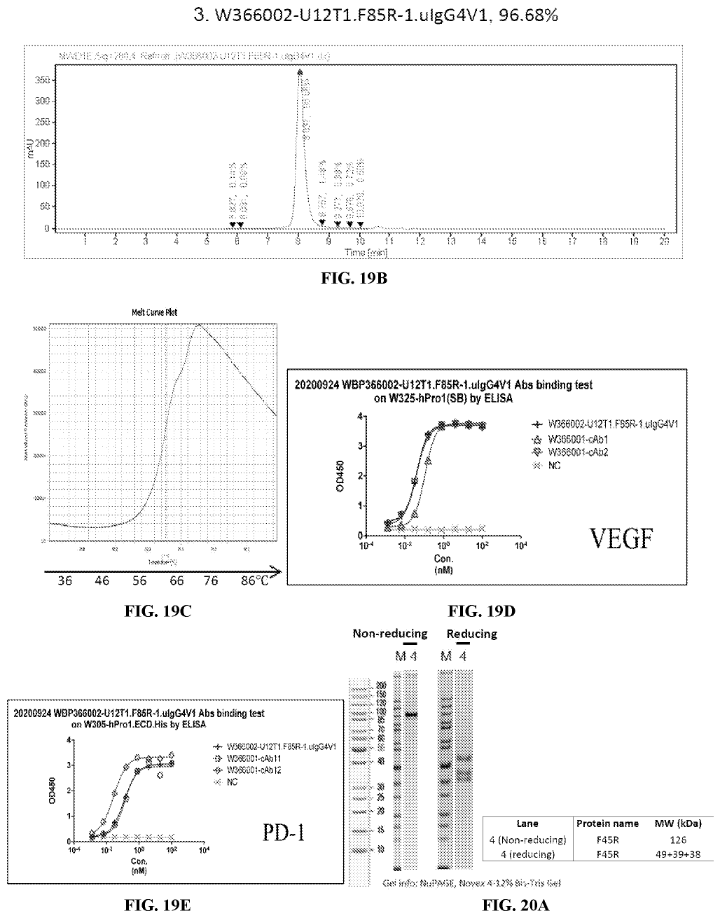

The schematic structure of the multispecific antibody F85R is shown in FIG. 16C. After purification, the purified F85R was analyzed by non-reducing and reducing gel electrophoresis, respectively. As shown in FIG. 19A, the results indicate that the F85R antibody was successfully purified with correct molecular weight. The SEC result is shown in FIG. 19B, in which a single major peak was observed. The final purity of the F85R antibody was determined as 96.68%. In addition, the Tₘ1 and Tₘ2 values of F85R were determined as 61.5° C. and 67.9° C., respectively, based on the melting curve shown in FIG. 19C.

In addition, the binding capabilities of F85R to VEGF and PD-1 were measured by ELISA, and the results are shown in FIG. 19D and FIG. 19E, respectively. The EC50 values of F85R binding to VEGF and PD-1 were determined as 0.040 μg/ml and 0.124 μg/ml, respectively.

### d) F45R

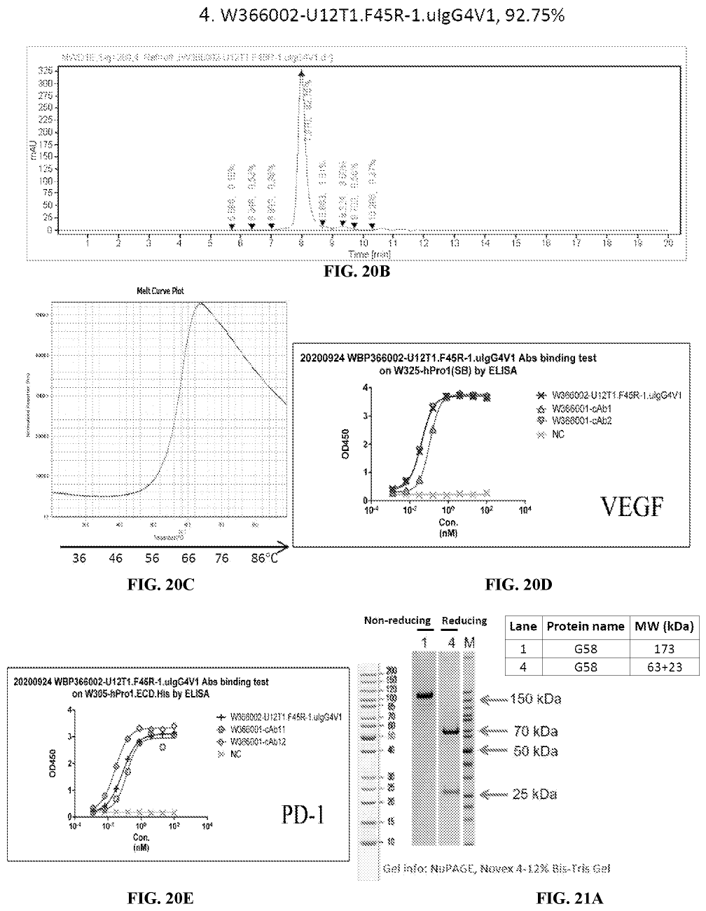

The schematic structure of the multispecific antibody F45R is shown in FIG. 16D. After purification, the purified F45R was analyzed by non-reducing and reducing gel electrophoresis, respectively. As shown in FIG. 20A, the results indicate that the F45R antibody was successfully purified with correct molecular weight. The SEC result is shown in FIG. 20B, in which a single major peak was observed. The final purity of the F45R antibody was determined as 92.75%. In addition, the Tₘ1 value of F45R was determined as 64.6° C., based on the melting curve shown in FIG. 20C.

In addition, the binding capabilities of F45R to VEGF and PD-1 were measured by ELISA, and the results are shown in FIG. 20D and FIG. 20E, respectively. The EC50 values of F45R binding to VEGF and PD-1 were determined as 0.042 μg/ml and 0.705 μg/ml, respectively.

### e) G58

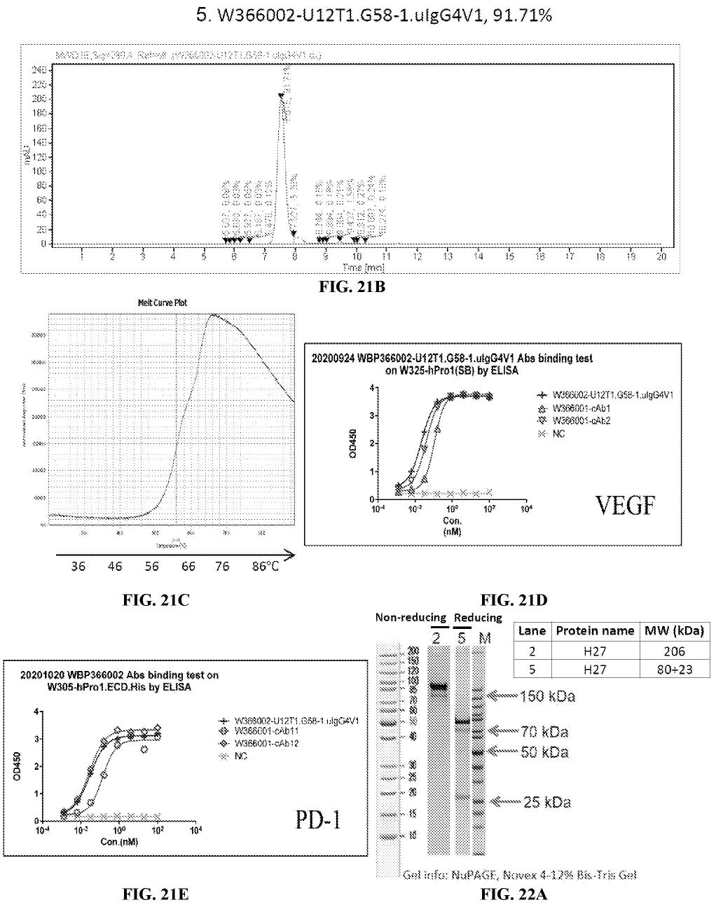

The schematic structure of the multispecific antibody G58 is shown in FIG. 16E. After purification, the purified G58 was analyzed by non-reducing and reducing gel electrophoresis, respectively. As shown in FIG. 21A, the results indicate that the G58 antibody was successfully purified with correct molecular weight. The SEC result is shown in FIG. 21B, in which a single major peak was observed. The final purity of the G58 antibody was determined as 91.71%. In addition, the Tₘ1 and Tₘ2 values of G58 were determined as 62.0° C. and 67.9° C., respectively, based on the melting curve shown in FIG. 21C.

In addition, the binding capabilities of G58 to VEGF and PD-1 were measured by ELISA, and the results are shown in FIG. 21D and FIG. 21E, respectively. The EC50 values of G58 binding to VEGF and PD-1 were determined as 0.222 μg/ml and 0.029 μg/ml, respectively.

### f) H27

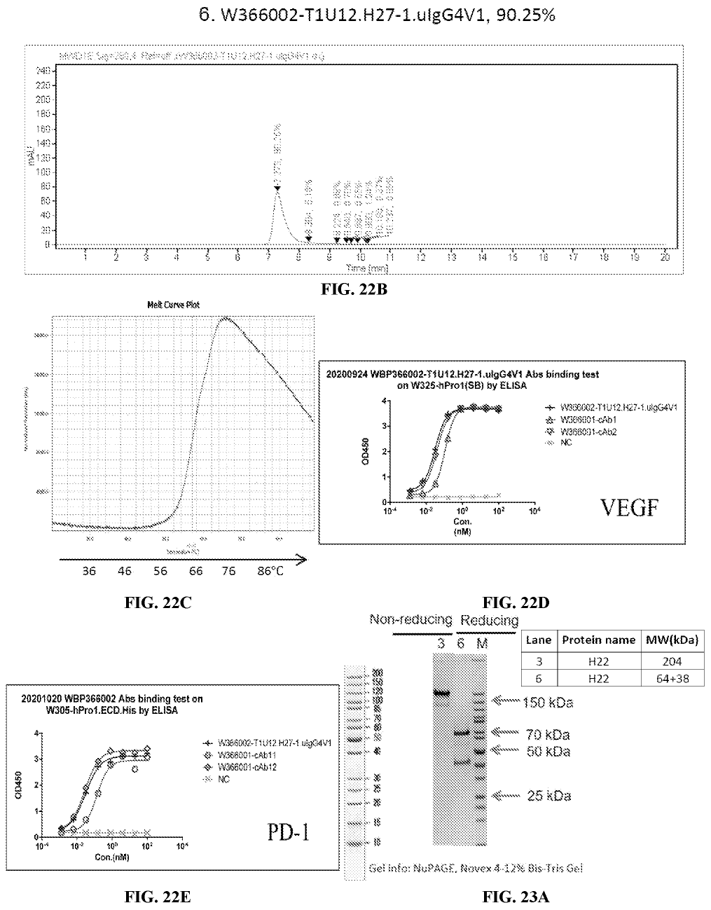

The schematic structure of the multispecific antibody H27 is shown in FIG. 16F. After purification step, the purified H27 was analyzed by non-reducing and reducing gel electrophoresis, respectively. As shown in FIG. 22A, the results indicate that the H27 antibody was successfully purified with correct molecular weight. The SEC result is shown in FIG. 22B, in which a single major peak was observed. The final purity of the H27 antibody was determined as 90.25%. In addition, the Tₘ1 and Tₘ2 values of H27 were determined as 62.9° C. and 67.2° C., respectively, based on the melting curve shown in FIG. 22C.

In addition, the binding capabilities of H27 to VEGF and PD-1 were measured by ELISA, and the results are shown in FIG. 22D and FIG. 22E, respectively. The EC50 values of H27 binding to VEGF and PD-1 were determined as 0.031 μg/ml and 0.031 μg/ml, respectively.

### g) H22

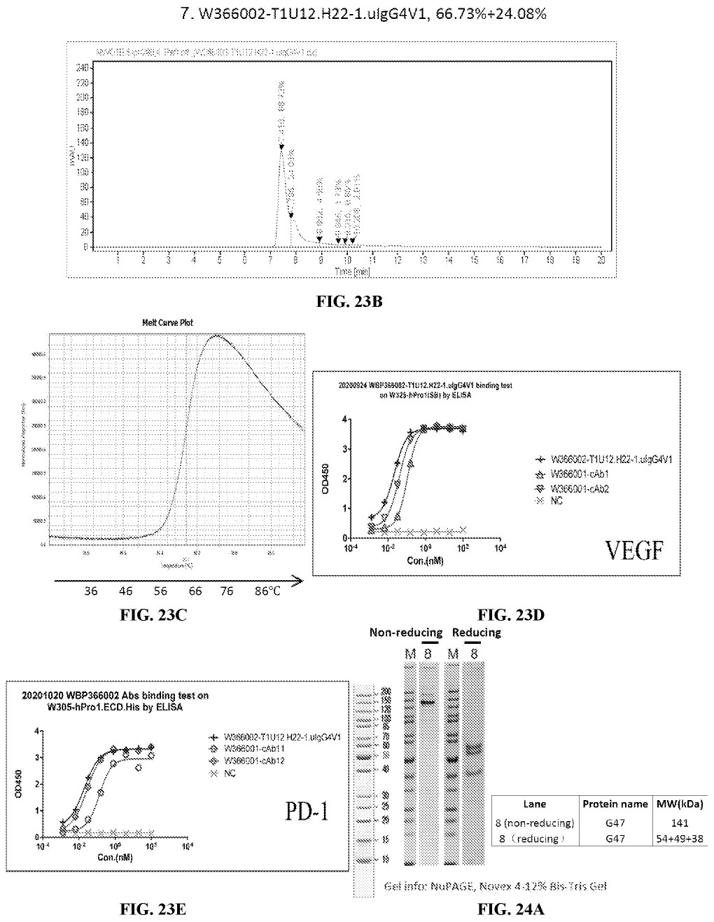

The schematic structure of the multispecific antibody H22 is shown in FIG. 16G. After purification, the purified H22 was analyzed by non-reducing and reducing gel electrophoresis, respectively. As shown in FIG. 23A, the results indicate that the H22 antibody was successfully purified with correct molecular weight. The SEC result is shown in FIG. 23B, in which a major peak and a minor peak were observed. The final purity of the H22 antibody was determined as 66.73% for the major peak and 24.08% for the minor peak. In addition, the Tₘ1 value of H22 was determined as 63.1° C., based on the melting curve shown in FIG. 23C.

In addition, the binding capabilities of H22 to VEGF and PD-1 were measured by ELISA, and the results are shown in FIG. 23D and FIG. 23E, respectively. The EC50 values of H22 binding to VEGF and PD-1 were determined as 0.021 μg/ml and 0.020 μg/ml, respectively.

### h) G47

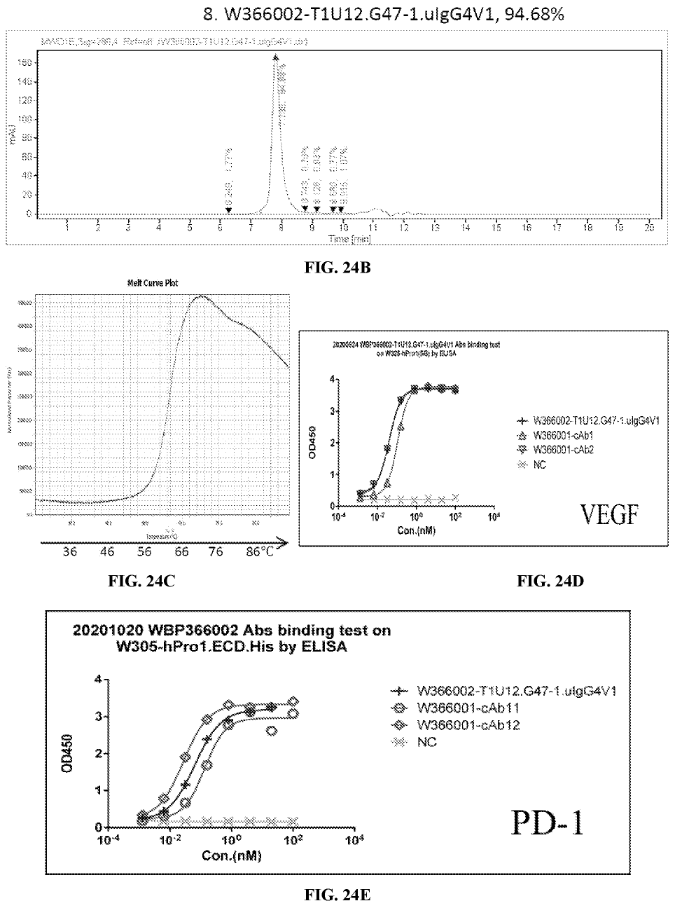

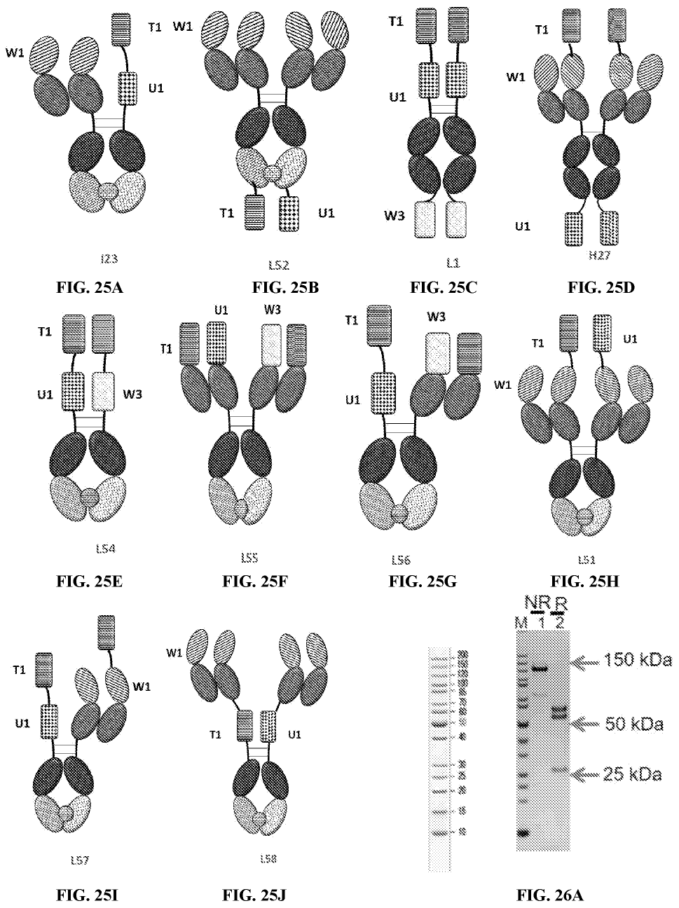

The schematic structure of the multispecific antibody G47 is shown in FIG. 16H. After purification, the purified G47 was analyzed by non-reducing and reducing gel electrophoresis, respectively. As shown in FIG. 24A, the results indicate that the G47 antibody was successfully purified with correct molecular weight. The SEC result is shown in FIG. 24B, in which a single major peak was observed. The final purity of the G47 antibody was determined as 94.68%. In addition, the Tₘ1 value of G47 was determined as 62.8° C., based on the melting curve shown in FIG. 24C.

In addition, the binding capabilities of G47 to VEGF and PD-1 were measured by ELISA, and the results are shown in FIG. 24D and FIG. 24E, respectively. The EC50 values of G47 binding to VEGF and PD-1 were determined as 0.040 μg/ml and 0.065 μg/ml, respectively.

### Example 5: Multispecific Antibodies Targeting VEGF, Ang-2, Mesothelin, and/or PD-1

Ten multispecific antibodies targeting VEGF, Ang-2, Mesothelin, and/or PD-1 were designed with schematic structures as shown in FIGS. 25A-25J. T1, U1, W3 represent VHHs targeting VEGF, Ang-2 and Mesothelin (MSLN), respectively. W1 represents a Fab domain targeting PD-1. The multispecific antibodies were named as W366003-T1U1W1.123-1.uIgG4V1 (or “123”), W366003-T1U1W1.L52-1.uIgG4V1 (or “L52”), W366003-T1U1W3.L1-1.uIgG4V1 (or “L1”), W366003-T1W1U1.H27-1.uIgG4V1 (or “H27-1”), W366003-T1U1W3.L54-1.uIgG4V1 (or “L54”), W366003-T1U1W3.L55-1.uIgG4V1 (or “L55”), W366003-T1U1W3.L56-1.uIgG4V1 (or “L56”), W366003-T1U1W1.L51-1.uIgG4V1 (or “L51”), W366003-T1U1W1.L57-1.uIgG4V1 (or “L57”), and W366003-T1U1W1.L58-1.uIgG4V1 (or “L58”), respectively.

The multispecific antibodies were purified by a protein A column. If the purity of samples was less than 90%, the samples were then further purified by the HPLC-SEC purification. The yield and purity after the purification were determined. The melting temperature (e.g., Tₘ1 and/or Tₘ2) was determined by DSF. ELISA was also performed to determine the binding affinity to the target. EC50 was calculated and was compared against the parental antibody. If the tested multispecific antibody is single valent for a target, a single valent parental antibody with the same binding site was selected for comparison purpose. If the tested multispecific antibody is multivalent (e.g., bivalent) for a target, a bivalent parental antibody with the same binding sites were selected for comparison purpose. The binding ratio (ratio of EC50) of each multispecific antibody relative to that of the parental antibody was also determined. The results are summarized in the table below.

TABLE 5

| | | Final | Final | | DSF | Binding Ratio | | | |
|---|---|---|---|---|---|---|---|---|---|
| | Protein | Yield | Purity | Purification | Tₘ1 | (Multispecific/Parental mAb) | | | |
| No. | Name | (mg/L) | SEC (%) | Steps | (° C.) | VEGF | Ang-2 | MSLN | PD-1 |
| 1 | I23 | 6.99 | 99.93% | 2 | 64.1 | 1.1 | 0.7 | / | 0.8 |
| 2 | L52 | 21.72 | 98.12% | 1 | 64.1 | 1.9 | 1.9 | / | 0.9 |
| 3 | L1 | 114.99 | 98.75% | 1 | 60.2 | 0.8 | 0.6 | 3.5 | / |
| 4 | H27-1 | 22.43 | 91.19% | 1 | 65.1 | 0.9 | 1.1 | / | 1.2 |
| 5 | L54 | 96.34 | 94.28% | 1 | 59.3 | 0.7 | 0.3 | 0.6 | / |
| 6 | L55 | 43.2 | 92.57% | 2 | 61.3 | 0.1 | 0.4 | 1.1 | / |
| 7 | L56 | 5.92 | 87.42% | 2 | 61.8 | 1.2 | 0.5 | 1.7 | / |
| 8 | L51 | 19.86 | 98.74% | 2 | 65.9 | 0.9 | 0.8 | / | 1.0 |
| 9 | L57 | 29.87 | 97.32% | 2 | 62.9 | 0.8 | 0.4 | / | 0.8 |
| 10 | L58 | 34.62 | 91.35% | 1 | 63.8 | 2.8 | 1.9 | / | 1.0 |

### a) 123 (VEGF/Ang-2/PD-1)

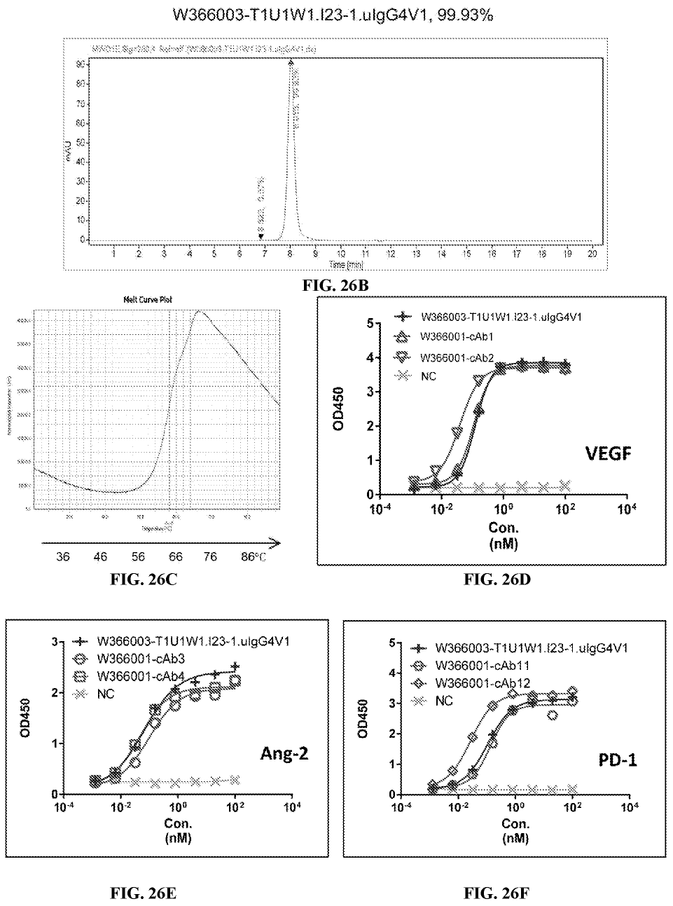

The schematic structure of the multispecific antibody 123 is shown in FIG. 25A. After purification, the purified 123 was analyzed by non-reducing (NR) and reducing (R) gel electrophoresis, respectively. As shown in FIG. 26A, the results indicate that the 123 antibody was successfully purified with correct molecular weight. The SEC result is shown in FIG. 26B, in which a single major peak was observed. The final purity of the 123 antibody was determined as 99.93%. In addition, the Tₘ1 value of 123 was determined as 64.1° C., based on the melting curve shown in FIG. 26C.

In addition, the binding capabilities of 123 to VEGF, Ang-2, and PD-1 were measured by ELISA, and the results are shown in FIGS. 26D-26F, respectively. The EC50 values of 123 binding to VEGF, Ang-2, and PD-1 were determined as 0.124 μg/ml, 0.067 μg/ml, and 0.106 μg/ml, respectively.

### b) L52 (VEGF/Ang-2/PD-1)

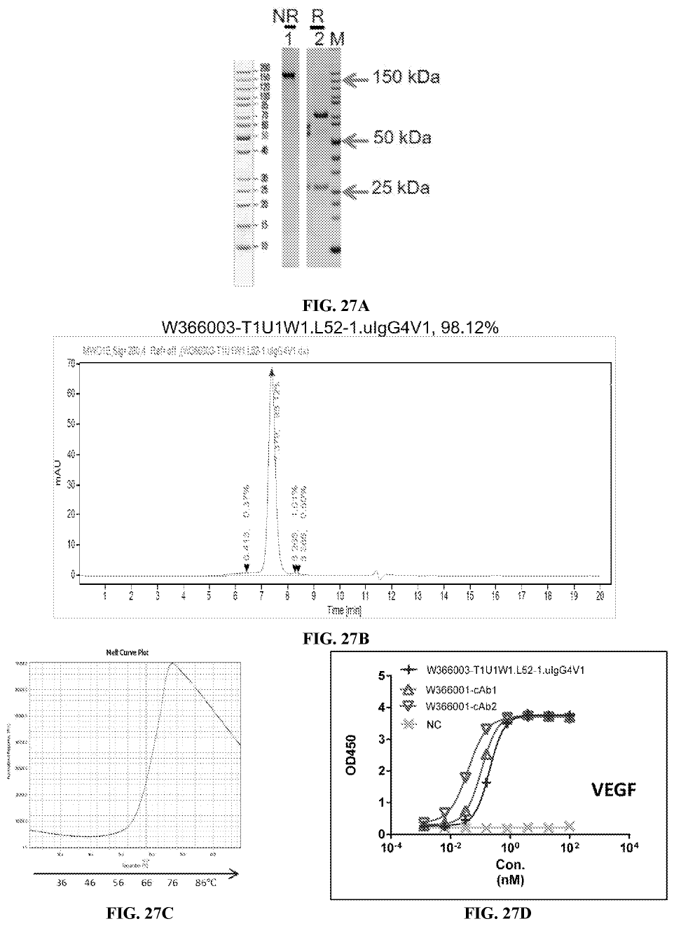

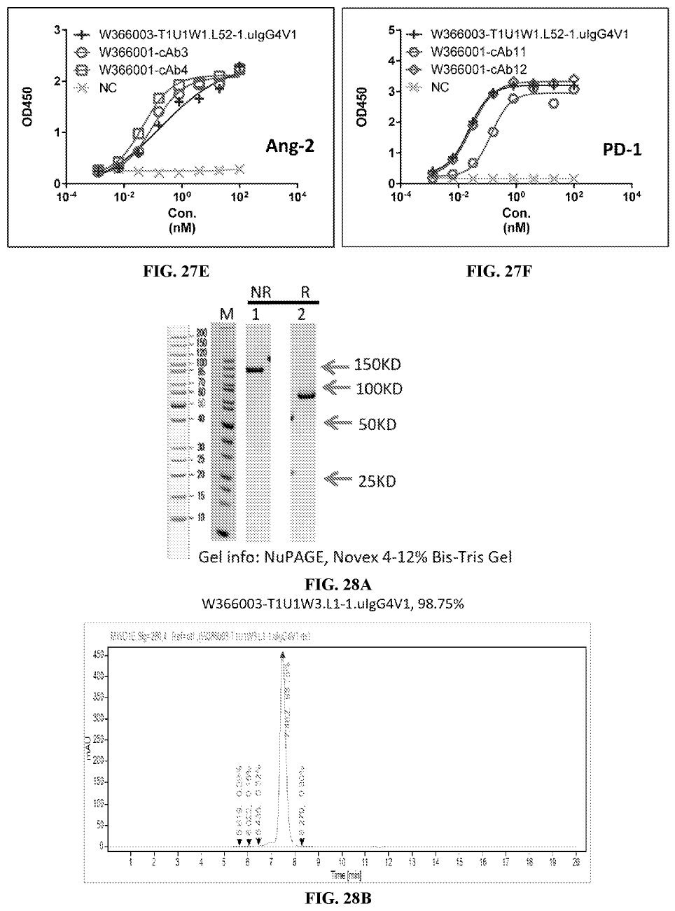

The schematic structure of the multispecific antibody L52 is shown in FIG. 25B. After purification, the purified L52 was analyzed by non-reducing (NR) and reducing (R) gel electrophoresis, respectively. As shown in FIG. 27A, the results indicate that the L52 antibody was successfully purified with correct molecular weight. The SEC result is shown in FIG. 27B, in which a single major peak was observed. The final purity of the L52 antibody was determined as 98.12%. In addition, the Tₘ1 value of L52 was determined as 64.1° C., based on the melting curve shown in FIG. 27C.

In addition, the binding capabilities of L52 to VEGF, Ang-2, and PD-1 were measured by ELISA, and the results are shown in FIGS. 27D-27F, respectively. The EC50 values of L52 binding to VEGF, Ang-2, and PD-1 were determined as 0.204 μg/ml, 0.190 μg/ml, and 0.023 μg/ml, respectively.

### c) L1 (VEGF/Ang-2/MSLN)

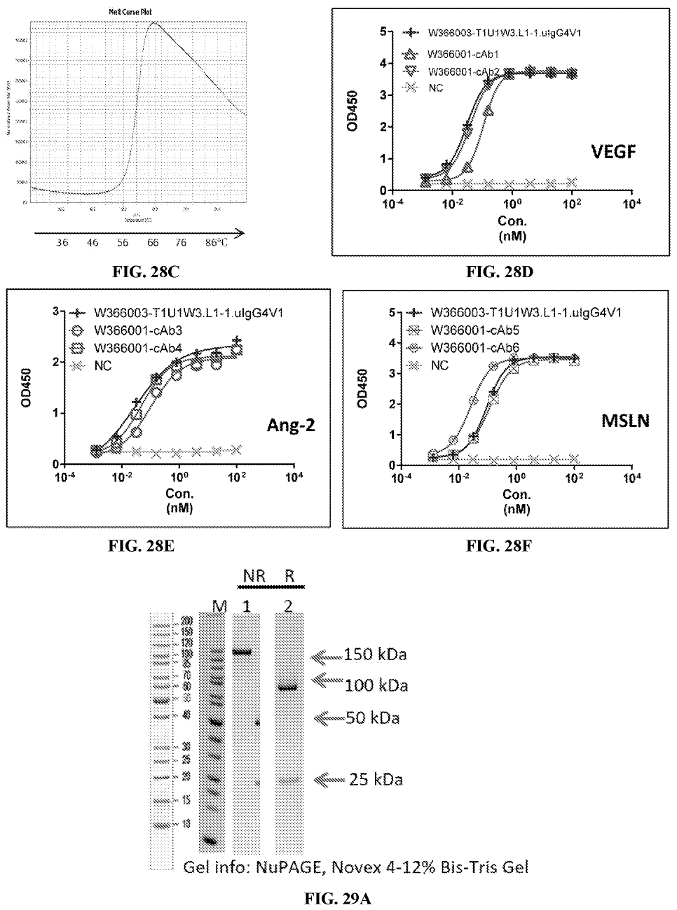

The schematic structure of the multispecific antibody L1 is shown in FIG. 25C. After purification, the purified L1 was analyzed by non-reducing (NR) and reducing (R) gel electrophoresis, respectively. As shown in FIG. 28A, the results indicate that the L1 antibody was successfully purified with correct molecular weight. The SEC result is shown in FIG. 28B, in which a single major peak was observed. The final purity of the L1 antibody was determined as 98.75%. In addition, the Tₘ1 value of L1 was determined as 60.2° C., based on the melting curve shown in FIG. 28C.

In addition, the binding capabilities of L1 to VEGF, Ang-2, and MSLN were measured by ELISA, and the results are shown in FIGS. 28D-28F, respectively. The EC50 values of L1 binding to VEGF, Ang-2, and MSLN were determined as 0.031 μg/ml, 0.027 μg/ml, and 0.092 μg/ml, respectively.

### d) H27-1 (VEGF/Ang-2/PD-1)

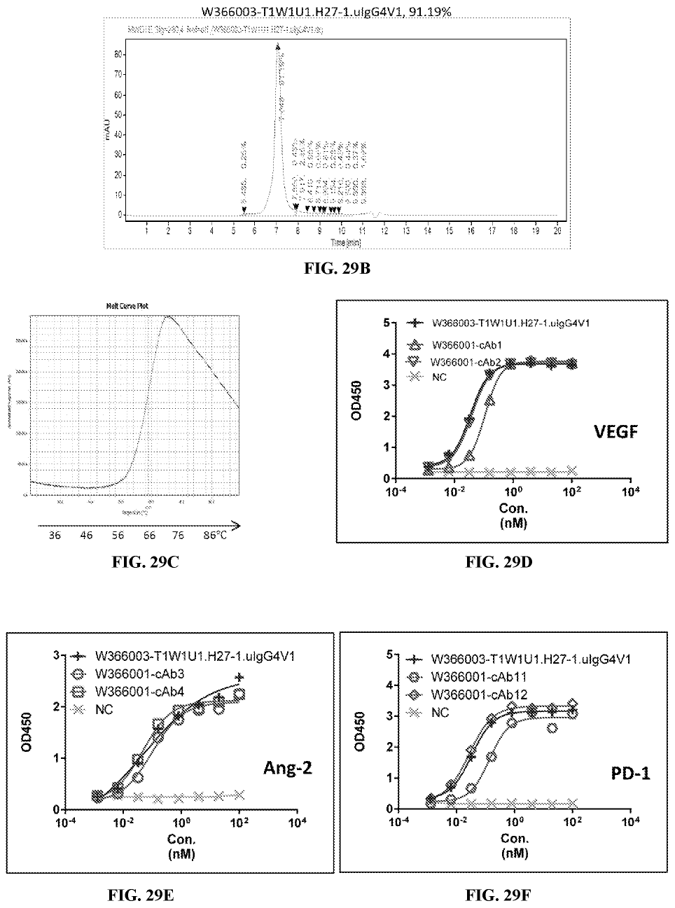

The schematic structure of the multispecific antibody H27-1 is shown in FIG. 25D. After purification, the purified H27-1 was analyzed by non-reducing (NR) and reducing (R) gel electrophoresis, respectively. As shown in FIG. 29A, the results indicate that the H27-1 antibody was successfully purified with correct molecular weight. The SEC result is shown in FIG. 29B, in which a single major peak was observed. The final purity of the H27-1 antibody was determined as 91.19%. In addition, the Tₘ1 value of H27-1 was determined as 65.1° C., based on the melting curve shown in FIG. 29C.

In addition, the binding capabilities of H27-1 to VEGF, Ang-2, and PD-1 were measured by ELISA, and the results are shown in FIGS. 29D-29F, respectively. The EC50 values of H27-1 binding to VEGF, Ang-2, and PD-1 were determined as 0.055 μg/ml, 0.052 μg/ml, and 0.033 μg/ml, respectively.

### e) L54 (VEGF/Ang-2/MSLN)

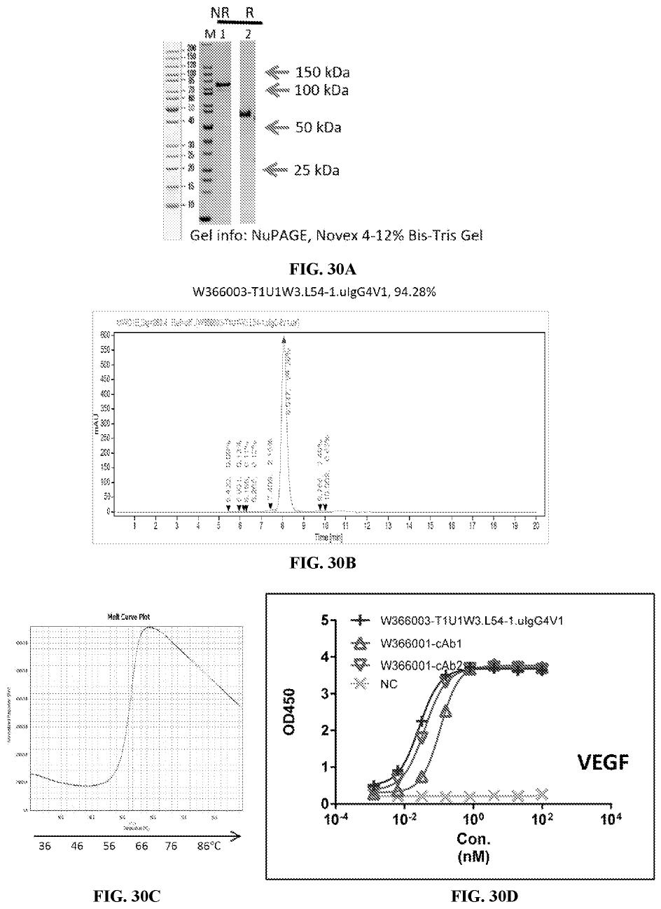

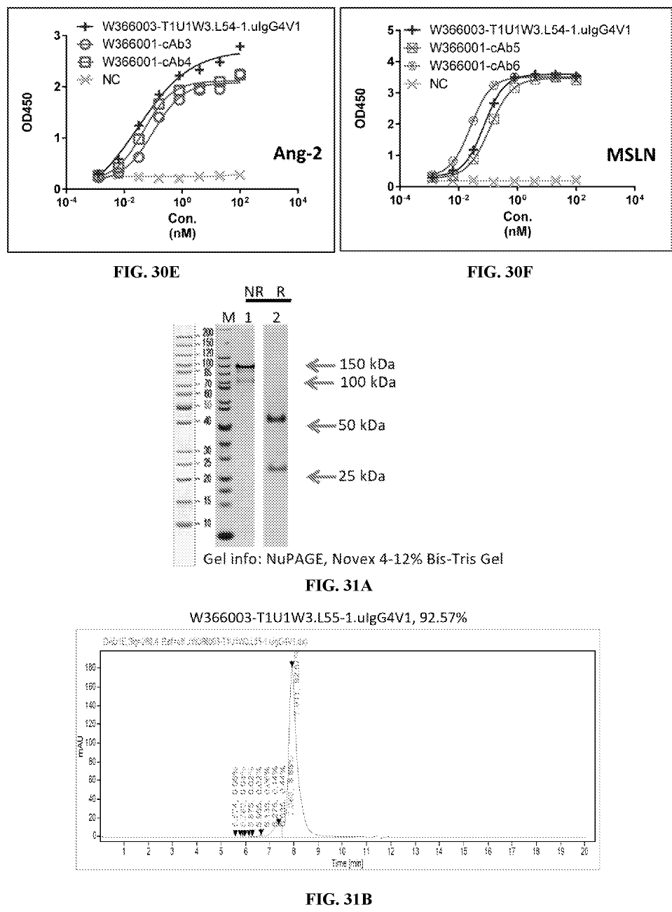

The schematic structure of the multispecific antibody L54 is shown in FIG. 25E. After purification, the purified L54 was analyzed by non-reducing (NR) and reducing (R) gel electrophoresis, respectively. As shown in FIG. 30A, the results indicate that the L54 antibody was successfully purified with correct molecular weight. The SEC result is shown in FIG. 30B, in which a single major peak was observed. The final purity of the L54 antibody was determined as 94.28%. In addition, the Tₘ1 value of L54 was determined as 59.3° C., based on the melting curve shown in FIG. 30C.

In addition, the binding capabilities of L54 to VEGF, Ang-2, and MSLN were measured by ELISA, and the results are shown in FIGS. 30D-30F, respectively. The EC50 values of L54 binding to VEGF, Ang-2, and MSLN were determined as 0.027 μg/ml, 0.030 μg/ml, and 0.073 μg/ml, respectively.

### f) L55 (VEGF/Ang-2/MSLN)

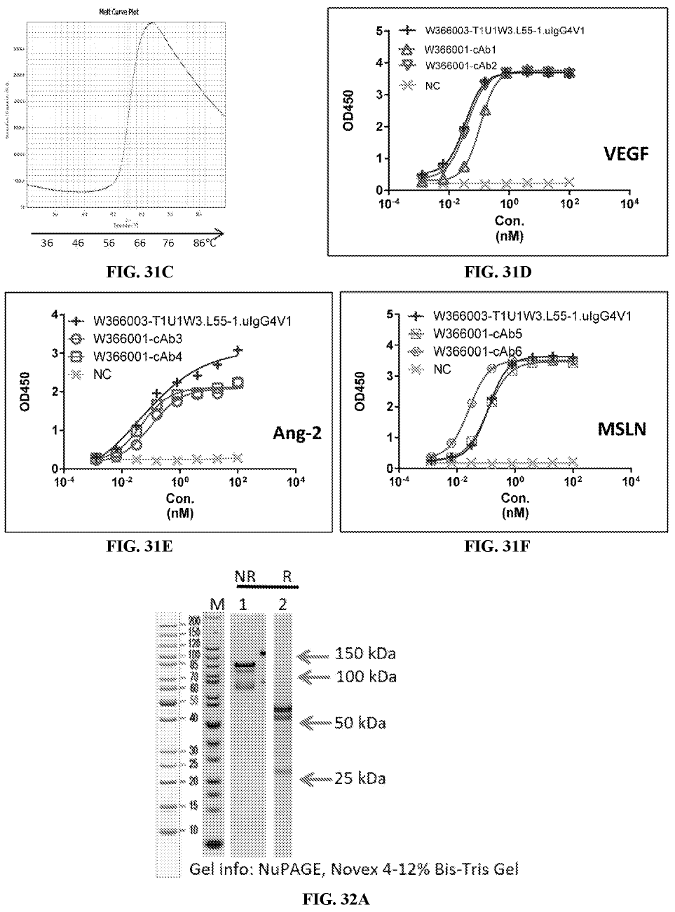

The schematic structure of the multispecific antibody L55 is shown in FIG. 25F. After purification, the purified L55 was analyzed by non-reducing (NR) and reducing (R) gel electrophoresis, respectively. As shown in FIG. 31A, the results indicate that the L55 antibody was successfully purified with correct molecular weight. The SEC result is shown in FIG. 31B, in which a single major peak was observed. The final purity of the L55 antibody was determined as 92.57%. In addition, the Tₘ1 value of L55 was determined as 61.3° C., based on the melting curve shown in FIG. 31C.

In addition, the binding capabilities of L55 to VEGF, Ang-2, and MSLN were measured by ELISA, and the results are shown in FIGS. 31D-31F, respectively. The EC50 values of L55 binding to VEGF, Ang-2, and MSLN were determined as 0.034 μg/ml, 0.043 μg/ml, and 0.121 μg/ml, respectively.

### g) L56 (VEGF/Ang-2/MSLN)

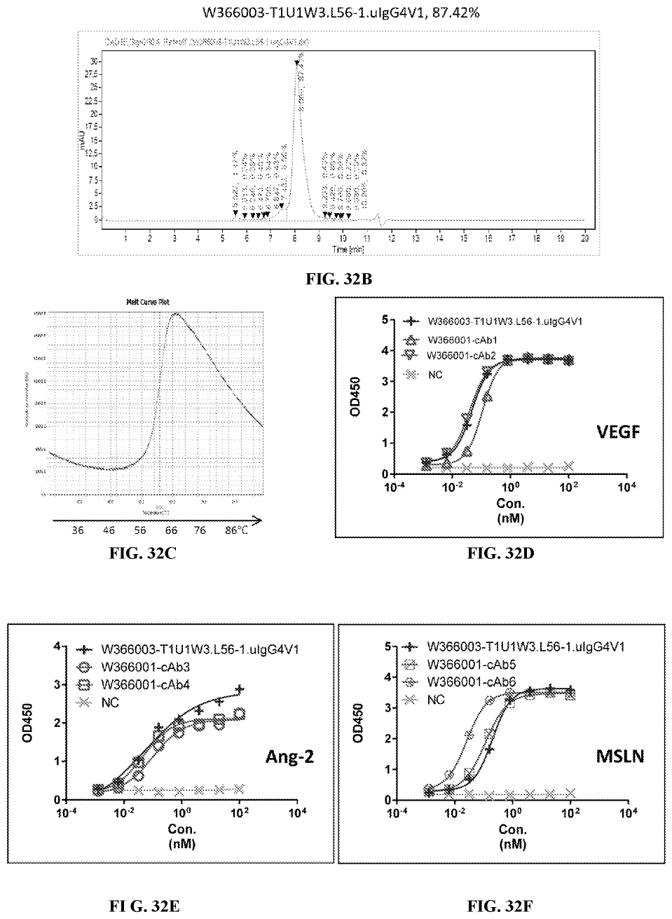

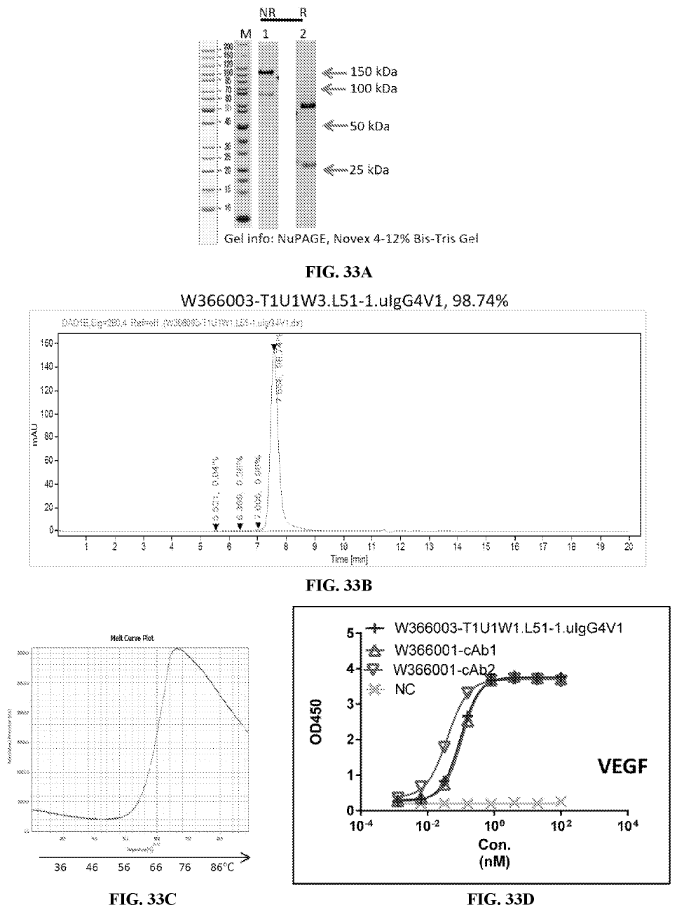

The schematic structure of the multispecific antibody L56 is shown in FIG. 25G. After purification, the purified L56 was analyzed by non-reducing (NR) and reducing (R) gel electrophoresis, respectively. As shown in FIG. 32A, the results indicate that the L56 antibody was successfully purified with correct molecular weight. The SEC result is shown in FIG. 32B, in which a single major peak was observed. The final purity of the L56 antibody was determined as 87.42%. In addition, the Tₘ1 value of L56 was determined as 61.8° C., based on the melting curve shown in FIG. 32C.

In addition, the binding capabilities of L56 to VEGF, Ang-2, and MSLN were measured by ELISA, and the results are shown in FIGS. 32D-32F, respectively. The EC50 values of L56 binding to VEGF, Ang-2, and MSLN were determined as 0.048 μg/ml, 0.053 μg/ml, and 0.200 μg/ml, respectively.

### h) L51 (VEGF/Ang-2/PD-1)

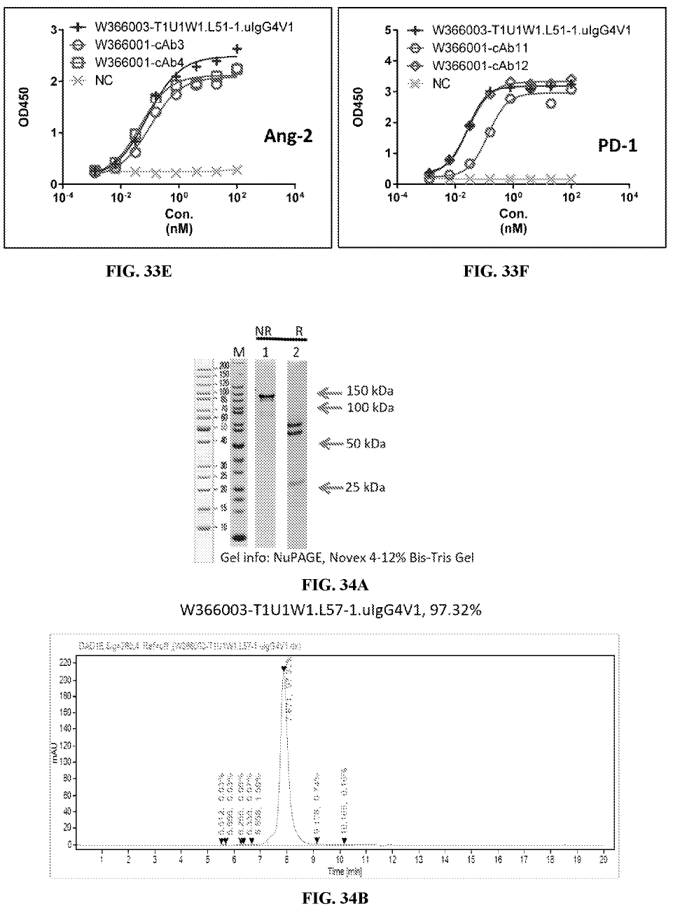

The schematic structure of the multispecific antibody L51 is shown in FIG. 25H. After purification, the purified L51 was analyzed by non-reducing (NR) and reducing (R) gel electrophoresis, respectively. As shown in FIG. 33A, the results indicate that the L51 antibody was successfully purified with correct molecular weight. The SEC result is shown in FIG. 33B, 5 in which a single major peak was observed. The final purity of the L51 antibody was determined as 98.74%. In addition, the Tₘ1 value of L51 was determined as 65.9° C., based on the melting curve shown in FIG. 33C.

In addition, the binding capabilities of L51 to VEGF, Ang-2, and PD-1 were measured by ELISA, and the results are shown in FIGS. 33D-33F, respectively. The EC50 values of L51 binding to VEGF, Ang-2, and PD-1 were determined as 0.095 μg/ml, 0.082 μg/ml, and 3.245 μg/ml, respectively.

### i) L57 (VEGF/Ang-2/PD-1)

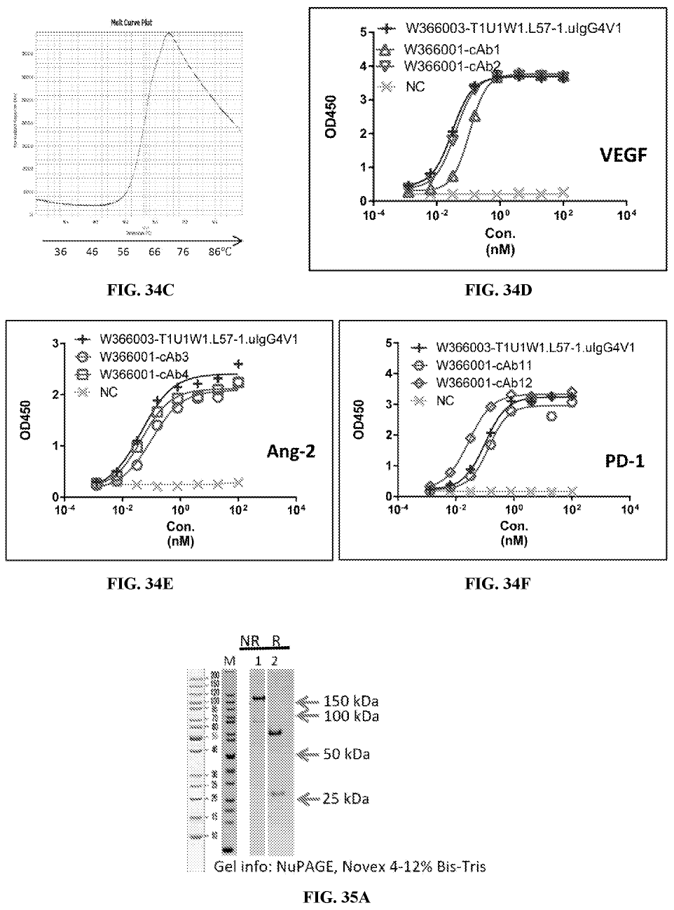

The schematic structure of the multispecific antibody L57 is shown in FIG. 25I. After purification, the purified L57 was analyzed by non-reducing (NR) and reducing (R) gel electrophoresis, respectively. As shown in FIG. 34A, the results indicate that the L57 antibody was successfully purified with correct molecular weight. The SEC result is shown in FIG. 34B, in which a single major peak was observed. The final purity of the L57 antibody was determined as 97.32%. In addition, the Tₘ1 value of L57 was determined as 62.9° C., based on the melting curve shown in FIG. 34C.

In addition, the binding capabilities of L57 to VEGF, Ang-2, and PD-1 were measured by ELISA, and the results are shown in FIGS. 34D-34F, respectively. The EC50 values of L57 binding to VEGF, Ang-2, and PD-1 were determined as 0.031 μg/ml, 0.044 μg/ml, and 0.102 μg/ml, respectively.

### j) L58 (VEGF/Ang-2/PD-1)

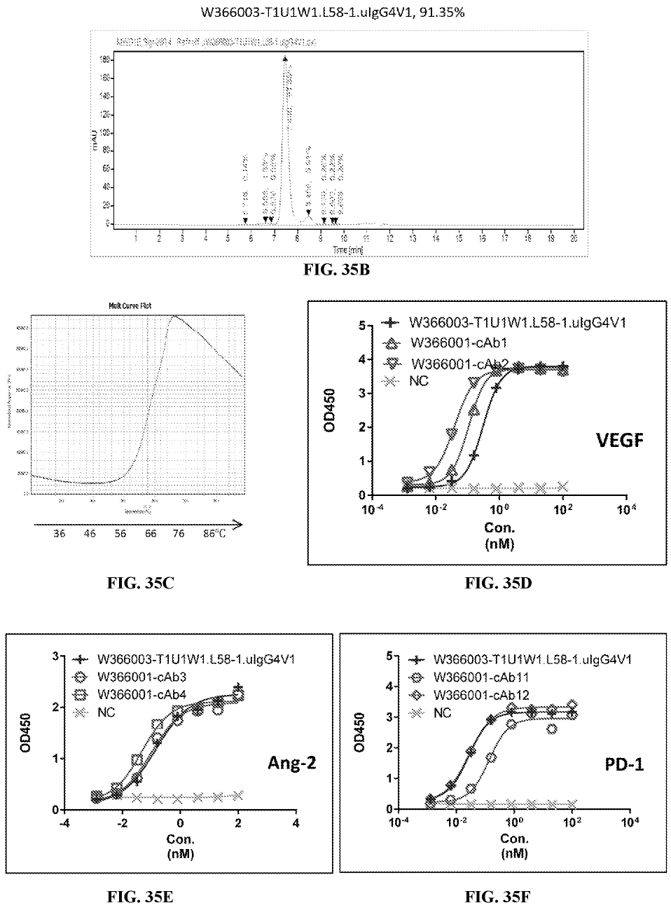

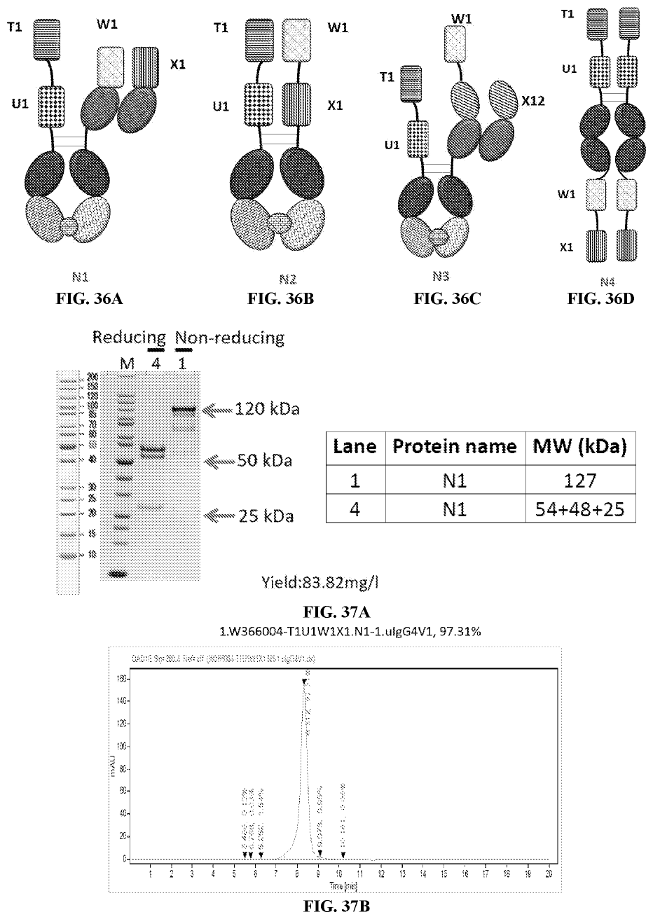

The schematic structure of the multispecific antibody L58 is shown in FIG. 25J. After purification, the purified L58 was analyzed by non-reducing (NR) and reducing (R) gel electrophoresis, respectively. As shown in FIG. 35A, the results indicate that the L58 antibody was successfully purified with correct molecular weight. The SEC result is shown in FIG. 35B, in which a single major peak was observed. The final purity of the L58 antibody was determined as 91.35%. In addition, the Tₘ1 value of L58 was determined as 63.8° C., based on the melting curve shown in FIG. 35C.

In addition, the binding capabilities of L58 to VEGF, Ang-2, and PD-1 were measured by ELISA, and the results are shown in FIGS. 35D-35F, respectively. The EC50 values of L58 binding to VEGF, Ang-2, and PD-1 were determined as 0.306 μg/ml, 0.145 μg/ml, and 0.027 μg/ml, respectively.

### Example 6: Multispecific Antibodies Targeting VEGF, Ang-2, Mesothelin, and GITR/PD-1

Ten multispecific antibodies targeting VEGF, Ang-2, Mesothelin, and GITR/PD-1 were designed with schematic structures as shown in FIGS. 36A-36D. T1, U1, and W1 represent VHHs targeting VEGF, Ang-2, and Mesothelin (MSLN), respectively. X1 represents a VHH targeting GITR. X12 represents a Fab domain targeting PD-1. The multispecific antibodies were named as W366004-T1U1W1X1.N1-1.uIgG4V1 (or “N1”), W366004-T1U1W1X1.N2-1.uIgG4V1 (or “N2”), W366004-T1U1W1X1.N3-1.uIgG4V1 (or “N3”), and W366004-T1U1 W1X1.N4-1.uIgG4V1 (or “N4”), respectively.

The multispecific antibodies were purified by a protein A column. If the purity of samples was less than 90%, the samples were then further purified by the HPLC-SEC purification. The yield and purity after the purification were determined. The melting temperature (e.g., Tₘ1 and/or Tₘ2) was determined by DSF. ELISA was also performed to determine the binding affinity to the target. EC50 was calculated and was compared against the parental antibody. If the tested multispecific antibody is single valent for a target, a single valent parental antibody with the same binding site was selected for comparison purpose. If the tested multispecific antibody is multivalent (e.g., bivalent) for a target, a bivalent parental antibody with the same binding sites were selected for comparison purpose. The binding ratio (ratio of EC50) of each multispecific antibody relative to that of the parental antibody was also determined. The results are summarized in the table below.

TABLE 6

| | | Final | Final | | DSF | Binding | Binding Ratio | | | |
|---|---|---|---|---|---|---|---|---|---|---|
| | Protein | Yield | Purity | Purification | Tₘ1 | ratio over | (Multispecific/Parental mAb) | | | |
| No. | Name | (mg/L) | SEC (%) | Steps | (° C.) | VEGF mAb | VEGF | Ang-2 | MSLN | GITR/PD-1 |
| 1 | N1 | 83.82 | 97% | 3 | 59.8 | 1.1 | 0.7 | 0.7 | 1.2 | 0.4 |
| 2 | N2 | 154.31 | 98% | 2 | 58.0 | 1.9 | 0.9 | 0.5 | 1.3 | 0.9 |
| 3 | N3 | 94.98 | 96% | 2 | 62.8 | 0.8 | 0.6 | 0.3 | 2.9 | 1.7 |
| 4 | N4 | 113.76 | 92% | 1 | 57.7 | 0.9 | 1.0 | 0.9 | 1.8 | 5.9 |

### a) N1 (VEGF/Ang-2/MSLN/GITR)

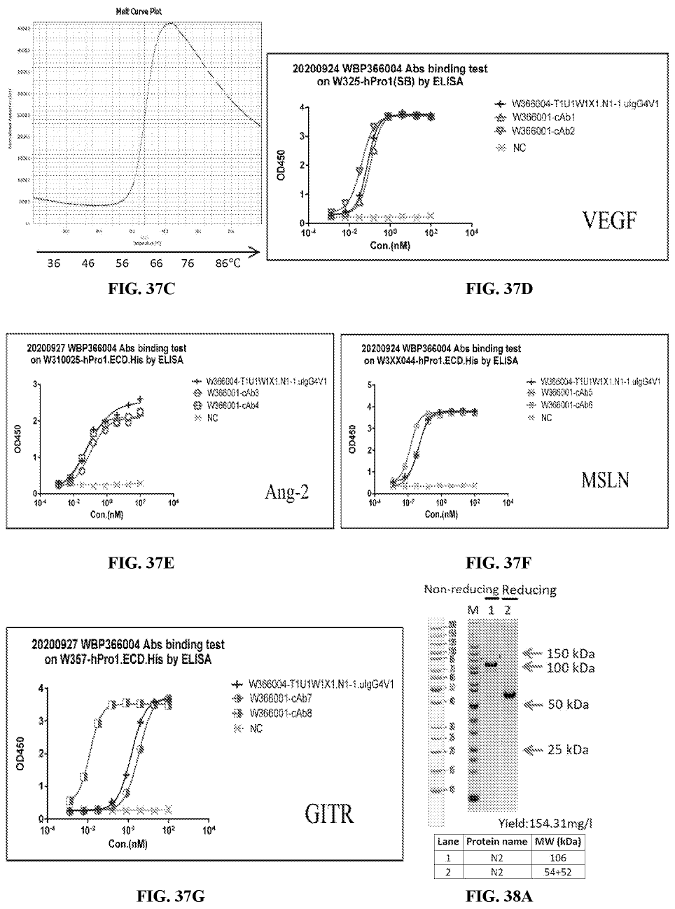

The schematic structure of the multispecific antibody N1 is shown in FIG. 36A. After purification, the purified N1 was analyzed by non-reducing (NR) and reducing (R) gel electrophoresis, respectively. As shown in FIG. 37A, the results indicate that the N1 antibody was successfully purified with correct molecular weight. The SEC result is shown in FIG. 37B, in which a single major peak was observed. The final purity of the N1 antibody was determined as 97.31%. In addition, the Tₘ1 value of N1 was determined as 63.1° C., based on the melting curve shown in FIG. 37C.

In addition, the binding capabilities of N1 to VEGF, Ang-2, Mesothelin, and GITR were measured by ELISA, and the results are shown in FIGS. 37D-37G, respectively.

### b) N2 (VEGF/Ang-2/MSLN/GITR)

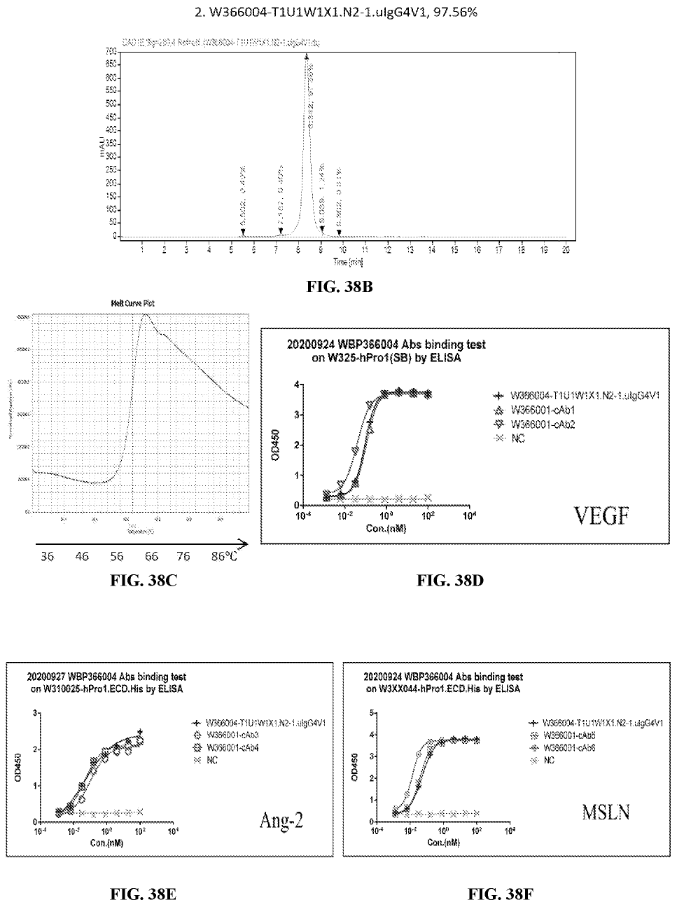

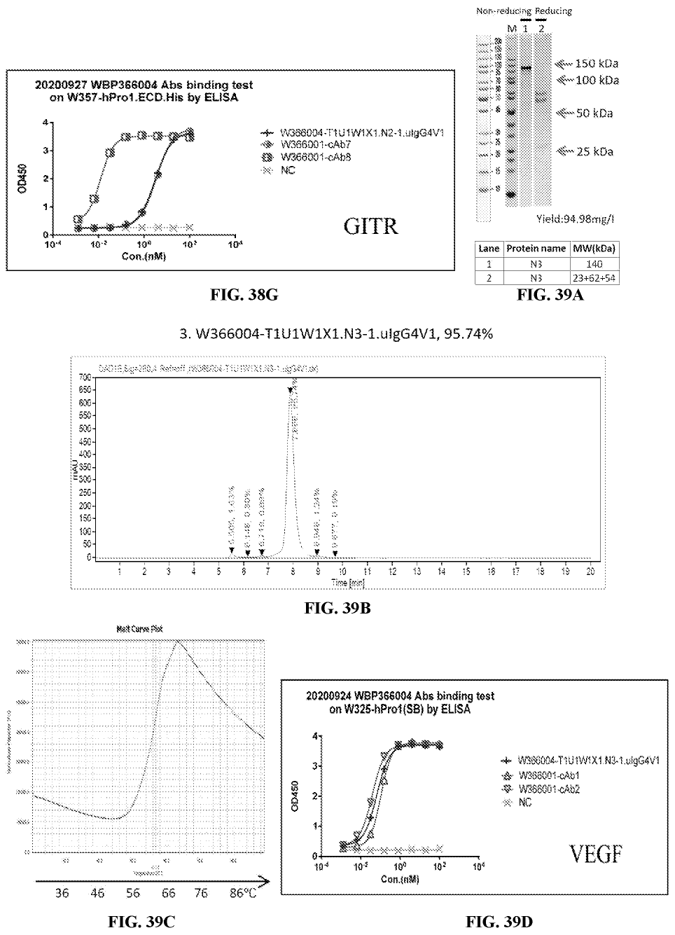

The schematic structure of the multispecific antibody N2 is shown in FIG. 36B. After purification, the purified N2 was analyzed by non-reducing (NR) and reducing (R) gel electrophoresis, respectively. As shown in FIG. 38A, the results indicate that the N2 antibody was successfully purified with correct molecular weight. The SEC result is shown in FIG. 38B, in which a single major peak was observed. The final purity of the N2 antibody was determined as 97.56%. In addition, the Tₘ1 and Tₘ2 values of N2 were determined as 58.0° C. and 67.7° C., respectively, based on the melting curve shown in FIG. 38C.

In addition, the binding capabilities of N2 to VEGF, Ang-2, Mesothelin, and GITR were measured by ELISA, and the results are shown in FIGS. 38D-38G, respectively.

### c) N3 (VEGF/Ang-2/MSLN/PD-1)

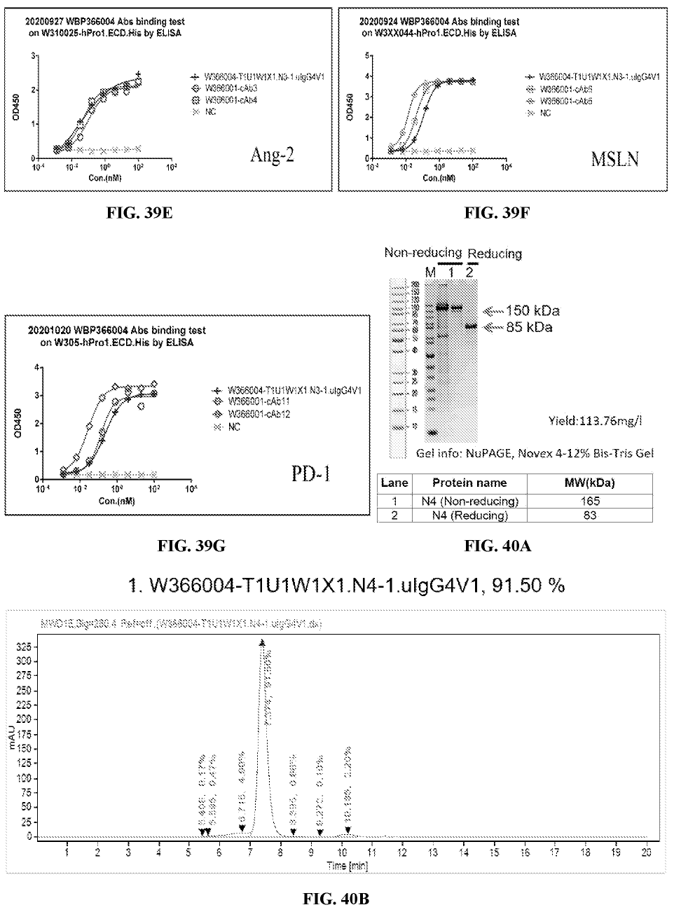

The schematic structure of the multispecific antibody N3 is shown in FIG. 36C. After purification, the purified N3 was analyzed by non-reducing (NR) and reducing (R) gel electrophoresis, respectively. As shown in FIG. 39A, the results indicate that the N3 antibody was successfully purified with correct molecular weight. The SEC result is shown in FIG. 39B, in which a single major peak was observed. The final purity of the N3 antibody was determined as 95.74%. In addition, the Tₘ1 and Tₘ2 values of N3 were determined as 62.8° C. and 67.5° C., respectively, based on the melting curve shown in FIG. 39C.

In addition, the binding capabilities of N3 to VEGF, Ang-2, Mesothelin, and PD-1 were measured by ELISA, and the results are shown in FIGS. 39D-39G, respectively.

### d) N4 (VEGF/Ang-2/MSLN/GITR)

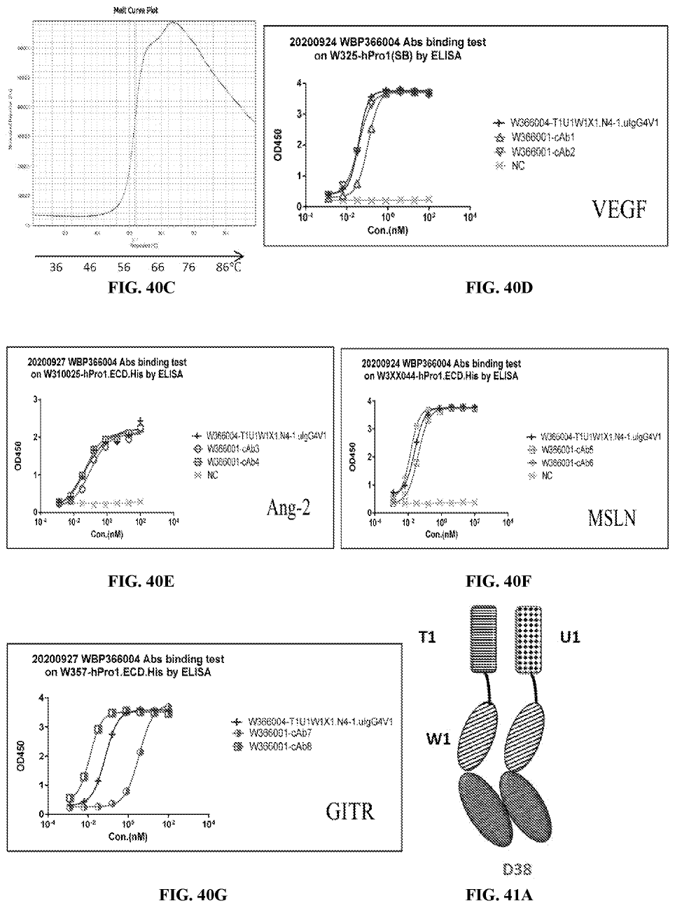

The schematic structure of the multispecific antibody N4 is shown in FIG. 36D. After purification, the purified N4 was analyzed by non-reducing (NR) and reducing (R) gel electrophoresis, respectively. As shown in FIG. 40A, the results indicate that the N4 antibody was successfully purified with correct molecular weight. The SEC result is shown in FIG. 40B, in which a single major peak was observed. The final purity of the N4 antibody was determined as 91.50%. In addition, the Tₘ1 and Tₘ2 values of N4 were determined as 57.7° C. and 67.2° C., respectively, based on the melting curve shown in FIG. 40C.

In addition, the binding capabilities of N4 to VEGF, Ang-2, Mesothelin, and GITR were measured by ELISA, and the results are shown in FIGS. 40D-40G, respectively.

### Example 7: Multispecific Antibodies Targeting VEGF, Ang-2, and PD-1

A multispecific antibodies targeting VEGF, Ang-2, and PD-1 was designed with schematic structure as shown in FIG. 41A. T1 and U1 represent VHHs targeting VEGF and Ang-2, respectively. W1 represents an Fab region targeting PD-1. The multispecific antibody was named as W366003-T1U1W1.D38-1.His (or “D38”).

D38 was purified as described above. After purification, the purified D38 was analyzed by non-reducing (NR) and reducing (R) gel electrophoresis, respectively. As shown in FIG. 41B, the results indicate that the D38 antibody was successfully purified with correct molecular weight. The SEC result is shown in FIG. 41C, in which a single major peak was observed. The final purity of the D38 antibody was determined as 99.01%. As shown in FIG. 41D, the melting curve was measured by DSF.

### Example 8: Monospecific Antibodies Targeting VEGF

Two monospecific antibodies targeting VEGF were designed with schematic structures as shown in FIGS. 43A-43B. T1 represents VHHs targeting VEGF. The monospecific antibodies were named as W366000-T1.V1-1.uIgG4V1 (or “V1”), and W366000-T1.V2-1.uIgG4V1 (or “V2”), respectively.

The monospecific antibodies were purified by a protein A column. If the purity of samples was less than 90%, the samples were then further purified by the HPLC-SEC purification. The yield and purity after the purification were determined. The melting temperature (e.g., Tₘ1 and/or Tₘ2) was determined by DSF. ELISA was also performed to determine the binding affinity to the target. EC50 was calculated and was compared against the parental antibody. If the tested monospecific antibody is single valent for a target, a single valent parental antibody with the same binding site was selected for comparison purpose. If the tested monospecific antibody is multivalent (e.g., bivalent) for a target, a bivalent parental antibody with the same binding sites were selected for comparison purpose. The binding ratio (ratio of EC50) of each monospecific antibody relative to that of the parental antibody was also determined. The results are summarized in the table below.

TABLE 7

| | | | | | | Binding Ratio | |
|---|---|---|---|---|---|---|---|
| | Pro- | Final | Final | Purifica- | DSF | (Monospecific/ | |
| | tein | Yield | Purity | tion | Tₘ1 | Parental mAb) | |
| No. | Name | (mg/L) | SEC (%) | Steps | (° C.) | VEGF | Ang-2 |
| 1 | V1 | 258.96 | 100 | 1 | 57.5 | 0.5 | / |
| 2 | V2 | 49.83 | 92.89 | 1 | 58.0 | 1.1 | / |

### a) V1 (VEGF)

The schematic structure of the monospecific antibody V1 is shown in FIG. 43A. After purification, the purified V1 was analyzed by non-reducing and reducing gel electrophoresis, respectively. As shown in FIG. 44A, the results indicate that the V1 antibody was successfully purified with correct molecular weight. The SEC analysis result is shown in FIG. 44B, in which a single major peak was observed. The final purity of the V1 antibody was determined as 100%. In addition, the Tₘ1 value of V1 was determined as 57.5° C., based on the melting curve shown in FIG. 44C.

The binding capability of V1 to VEGF was measured by ELISA, and the result is shown in FIG. 44D. The EC50 values of V1 binding to VEGF was determined as 0.0434 μg/ml.

### b) V2 (VEGF)

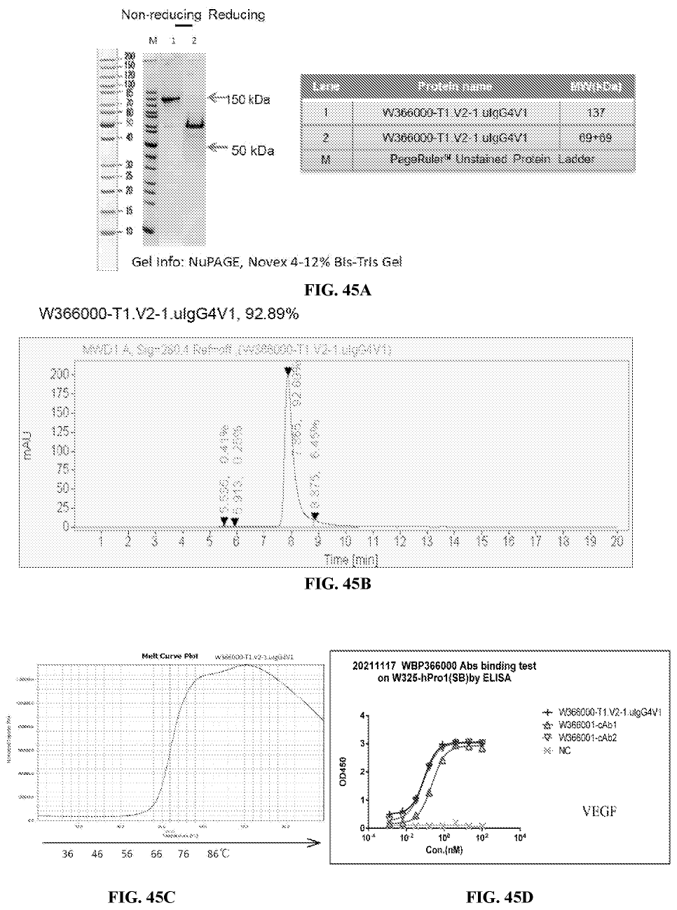

The schematic structure of the monospecific antibody V2 is shown in FIG. 43B. After purification, the purified V2 was analyzed by non-reducing and reducing gel electrophoresis, respectively. As shown in FIG. 45A, the results indicate that the V2 antibody was successfully purified with correct molecular weight. The SEC analysis result is shown in FIG. 45B, in which a single major peak was observed. The final purity of the V2 antibody was determined as 92.89%. In addition, the Tₘ1 and Tₘ2 value of V2 was determined as 58.0° C. and 62.8° C., respectively, based on the melting curve shown in FIG. 45C.

The binding capability of V2 to VEGF was measured by ELISA, and the result is shown in FIG. 45D. The EC50 values of V2 binding to VEGF was determined as 0.0876 μg/ml.

### Example 9: Multispecific Antibodies Targeting VEGF and Ang-2

Six multispecific antibodies targeting VEGF and Ang-2 were designed with schematic structures as shown in FIGS. 46A-46F. T1 and U1 represent VHHs targeting VEGF and Ang-2, respectively. The multispecific antibodies were named as W366001-U1T1.H39-1.uIgG4V1 (or “H39”), W366001-U1T1.H40-1.uIgG4V1 (or “H40”), W366001-U1 T1.V14-1. His (or “V14”), W366001-U1T1.V15-1.His (or “V15”), W366001-U1T1.V16-1.His (or “V16”), and W366001-U1T1.V11-1.His (or “V11”), respectively.

The multispecific antibodies were purified by a protein A column. If the purity of samples was less than 90%, the samples were then further purified by the HPLC-SEC purification. The yield and purity after the purification were determined. The melting temperature (e.g., Tₘ1 and/or Tₘ2) was determined by DSF. ELISA was also performed to determine the binding affinity to the target. EC50 was calculated and was compared against the parental antibody. If the tested multispecific antibody is single valent for a target, a single valent parental antibody with the same binding site was selected for comparison purpose. If the tested multispecific antibody is multivalent (e.g., bivalent) for a target, a bivalent parental antibody with the same binding sites were selected for comparison purpose. The binding ratio (ratio of EC50) of each multispecific antibody relative to that of the parental antibody was also determined. The results are summarized in the table below.

TABLE 8

| | | | | | | Binding Ratio | |
|---|---|---|---|---|---|---|---|
| | Pro- | Final | Final | Purifica- | DSF | (Monospecific/ | |
| | tein | Yield | Purity | tion | Tₘ1 | Parental mAb) | |
| No. | Name | (mg/L) | SEC (%) | Steps | (° C.) | VEGF | Ang-2 |
| 1 | H39 | 326.90 | 99.28 | 1 | 58.2 | 0.66 | 2.3 |
| 2 | H40 | 76.75 | 97.35 | 2 | 60.5 | 1.4 | 1.6 |
| 3 | V14 | 66.89 | 90.58 | 1 | 62.3 | / | / |
| 4 | V15 | 206.50 | 92.31 | 1 | 59.0 | / | / |
| 5 | V16 | 67.33 | 99.47 | 2 | 58.4 | / | / |
| 6 | V11 | 57.65 | 99.05 | 2 | 61.3 | / | / |

### a) H39 (VEGF/Ang-2)

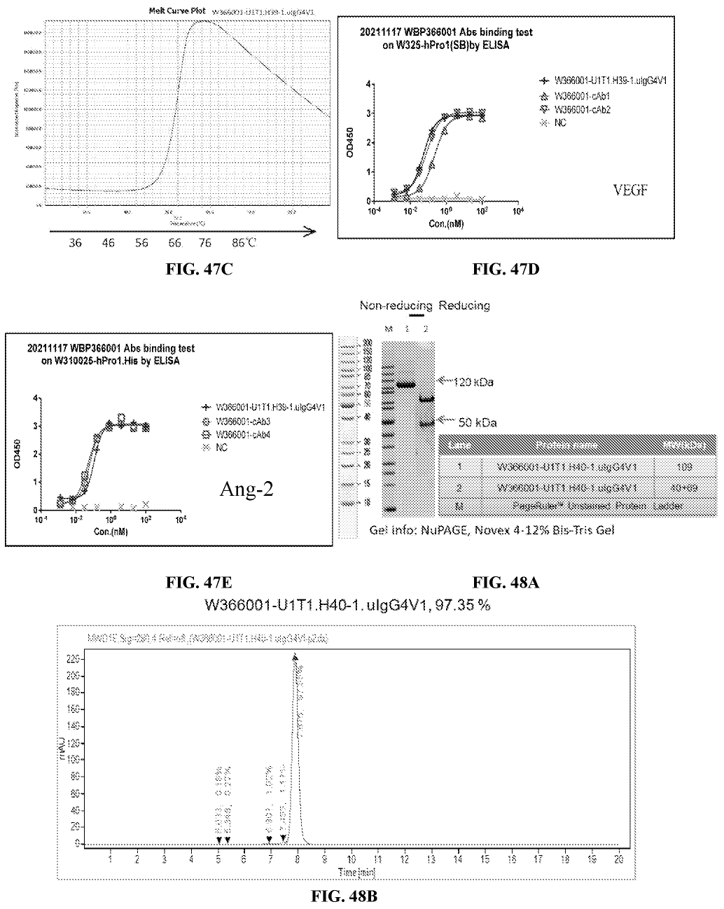

The schematic structure of the multispecific antibody H39 is shown in FIG. 46A. After purification, the purified H39 was analyzed by non-reducing (N) and reducing (R) gel electrophoresis, respectively. As shown in FIG. 47A, multiple non-target bands were observed in the purified H39 antibody sample. The SEC analysis result is shown in FIG. 47B, in which a single major peak was observed. The final purity of the H39 antibody was determined as 99.28%. In addition, the Tₘ1 value of H39 was determined as 58.2° C., based on the melting curve shown in FIG. 47C.

The binding capabilities of H39 to VEGF and Ang-2 were measured by ELISA, and the results are shown in FIG. 47D and FIG. 47E, respectively. The EC50 values of H39 binding to VEGF and Ang-2 were determined as 0.0528 μg/ml and 0.1114 μg/ml, respectively.

### b) H40 (VEGF/Ang-2)

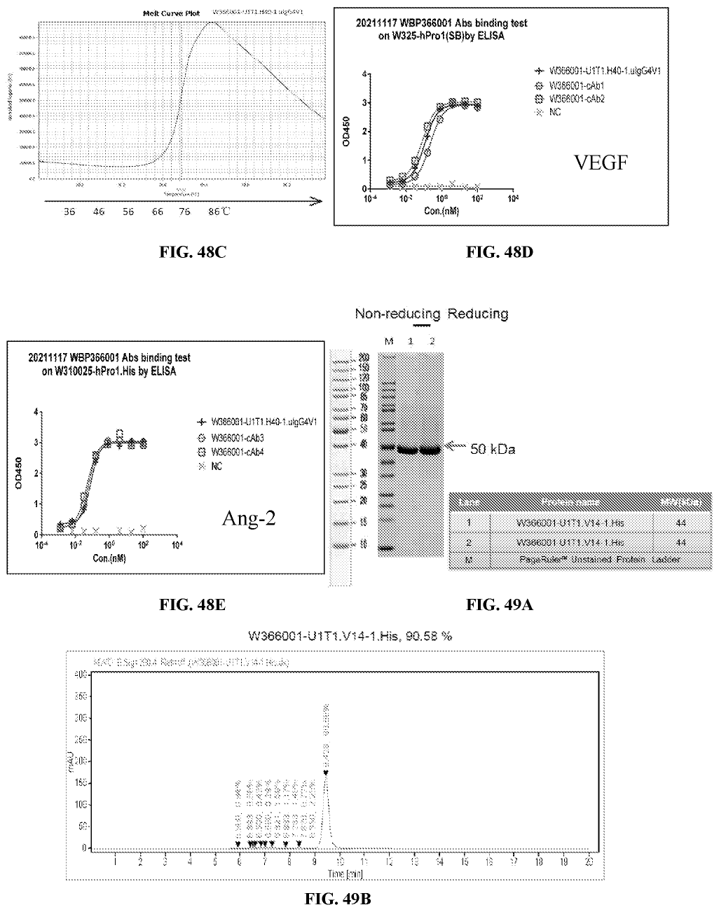

The schematic structure of the multispecific antibody H40 is shown in FIG. 46B. After purification, the purified H40 was analyzed by non-reducing (N) and reducing (R) gel electrophoresis, respectively. As shown in FIG. 48A, multiple non-target bands were observed in the purified H40 antibody sample. The SEC analysis result is shown in FIG. 48B, in which a single major peak was observed. The final purity of the H40 antibody was determined as 97.35%. In addition, the Tₘ1 value and Tₘ2 value of H40 were determined as 60.5° C. and 64.4° C., respectively, based on the melting curve shown in FIG. 48C.

The binding capabilities of H40 to VEGF and Ang-2 were measured by ELISA, and the results are shown in FIG. 48D and FIG. 48E, respectively. The EC50 values of H40 binding to VEGF and Ang-2 were determined as 0.0528 μg/ml and 0.1114 μg/ml, respectively.

### c) V14 (VEGF/Ang-2)

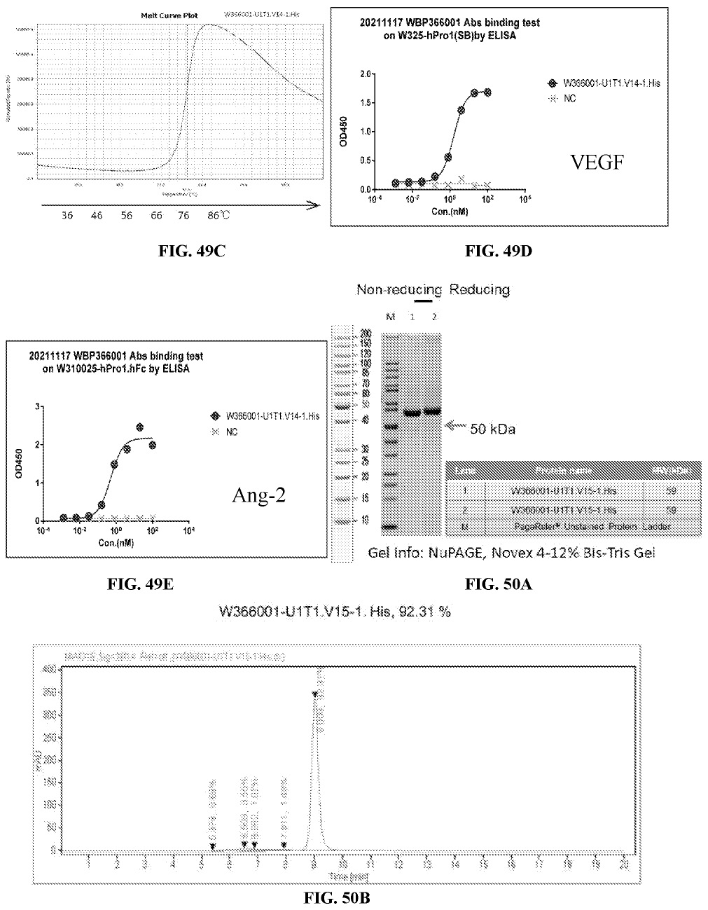

The schematic structure of the multispecific antibody V14 is shown in FIG. 46C. After purification, the purified V14 was analyzed by non-reducing (N) and reducing (R) gel electrophoresis, respectively. As shown in FIG. 49A, multiple non-target bands were observed in the purified V14 antibody sample. The SEC analysis result is shown in FIG. 49B, in which a single major peak was observed. The final purity of the V14 antibody was determined as 90.58%. In addition, the Tₘ1 value of V14 was determined as 62.3° C., based on the melting curve shown in FIG. 49C.

The binding capabilities of V14 to VEGF and Ang-2 were measured by ELISA, and the results are shown in FIG. 49D and FIG. 49E, respectively. The EC50 values of V14 binding to VEGF and Ang-2 were determined as 1.5680 μg/ml and 0.5059 μg/ml, respectively.

### d) V15 (VEGF/Ang-2)

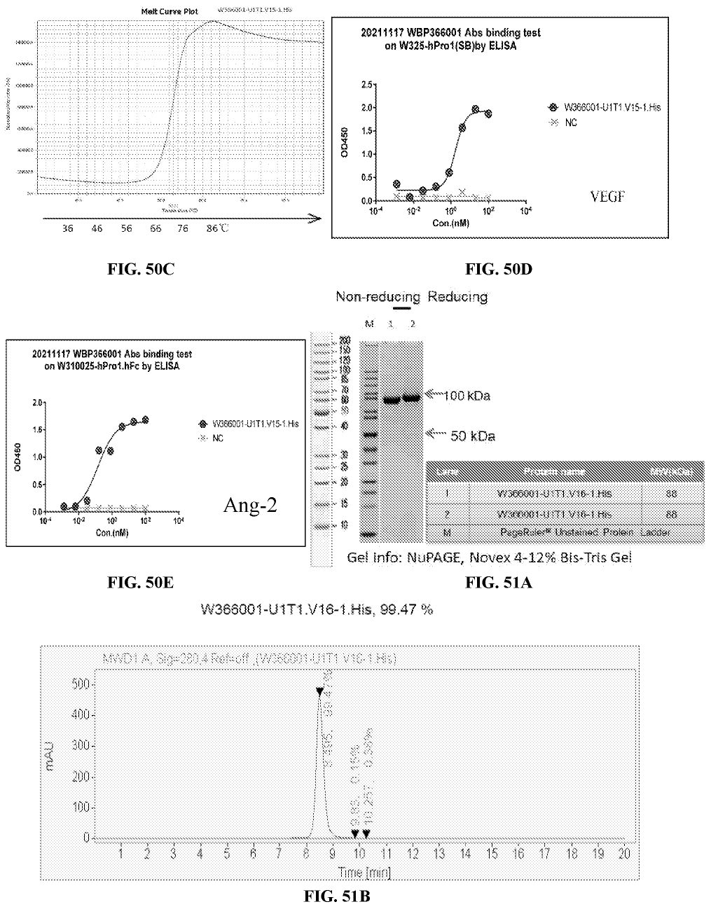

The schematic structure of the multispecific antibody V15 is shown in FIG. 46D. After purification, the purified V15 was analyzed by non-reducing (N) and reducing (R) gel electrophoresis, respectively. As shown in FIG. 50A, multiple non-target bands were observed in the purified V15 antibody sample. The SEC analysis result is shown in FIG. 50B, in which a single major peak was observed. The final purity of the V15 antibody was determined as 92.31%. In addition, the Tₘ1 value and Tₘ2 value were determined as 59.0° C. and 64.7° C., respectively, based on the melting curve shown in FIG. 50C.

The binding capabilities of V15 to VEGF and Ang-2 were measured by ELISA, and the results are shown in FIG. 50D and FIG. 50E, respectively. The EC50 values of V15 binding to VEGF and Ang-2 were determined as 1.7380 μg/ml and 0.1380 μg/ml, respectively.

### e) V16 (VEGF/Ang-2)

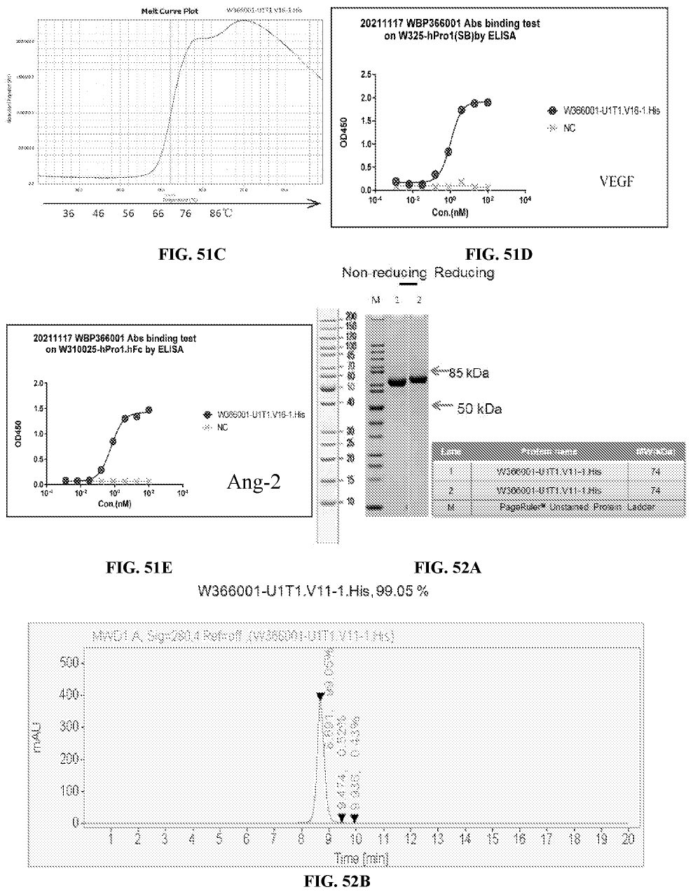

The schematic structure of the multispecific antibody V16 is shown in FIG. 46E. After purification, the purified V16 was analyzed by non-reducing (N) and reducing (R) gel electrophoresis, respectively. As shown in FIG. 51A, multiple non-target bands were observed in the purified V16 antibody sample. The SEC analysis result is shown in FIG. 51B, in which a single major peak was observed. The final purity of the V16 antibody was determined as 99.47%. In addition, the Tₘ1 value and Tₘ2 value were determined as 58.4° C. and 71.5° C., respectively, based on the melting curve shown in FIG. 51C.

The binding capabilities of V16 to VEGF and Ang-2 were measured by ELISA, and the results are shown in FIG. 51D and FIG. 51E, respectively. The EC50 values of V16 binding to VEGF and Ang-2 were determined as 1.0560 μg/ml and 0.6202 μg/ml, respectively.

### f) V11 (VEGF/Ang-2)

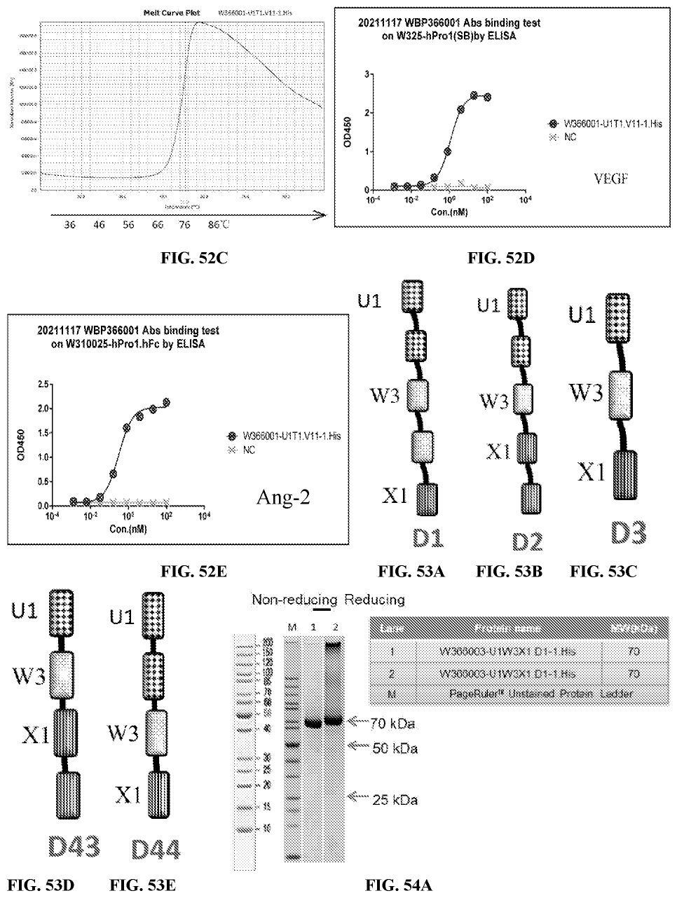

The schematic structure of the multispecific antibody V11 is shown in FIG. 46F. After purification, the purified V11 was analyzed by non-reducing (N) and reducing (R) gel electrophoresis, respectively. As shown in FIG. 52A, multiple non-target bands were observed in the purified V11 antibody sample. The SEC analysis result is shown in FIG. 52B, in which a single major peak was observed. The final purity of the V11 antibody was determined as 99.05%. In addition, the Tₘ1 value of V11 was determined as 61.3° C., based on the melting curve shown in FIG. 52C.

The binding capabilities of V11 to VEGF and Ang-2 were measured by ELISA, and the results are shown in FIG. 52D and FIG. 52E, respectively. The EC50 values of V11 binding to VEGF and Ang-2 were determined as 1.1350 μg/ml and 0.3108 μg/ml, respectively.

### Example 10: Multispecific Antibodies Targeting Ang-2, Mesothelin, and GITR

Five multispecific antibodies targeting Ang-2, Mesothelin, and GITR were designed with schematic structures as shown in FIGS. 53A-53E. U1 and W3 represent VHHs targeting Ang-2 and Mesothelin (MSLN), respectively. X1 represents a VHH targeting GITR. The multispecific antibodies were named as W366003-U1W3X1.D1-1.His (or “D1”), W366003-U1W3X1.D2-1.His (or “D2”), W366003-U1W3X1.D3-1.His (or “D3”), W366003-U1W3X1.D43-1.His (or “D43”) and W366003-U1W3X1.D44-1.His (or “D44”), respectively.

The multispecific antibodies were purified by a protein A column. If the purity of samples was less than 90%, the samples were then further purified by the HPLC-SEC purification. The yield and purity after the purification were determined. The melting temperature (e.g., Tₘ1 and/or Tₘ2) was determined by DSF. ELISA was also performed to determine the binding affinity to the target. EC50 was calculated and was compared against the parental antibody. If the tested multispecific antibody is single valent for a target, a single valent parental antibody with the same binding site was selected for comparison purpose. If the tested multispecific antibody is multivalent (e.g., bivalent) for a target, a bivalent parental antibody with the same binding sites were selected for comparison purpose. The binding ratio (ratio of EC50) of each multispecific antibody relative to that of the parental antibody was also determined. The results are summarized in the table below.

TABLE 9

| | | Final | Final | | |
|---|---|---|---|---|---|
| | Protein | Yield | Purity | Purification | DSF Tₘ1 |
| No. | Name | (mg/L) | SEC (%) | Steps | (° C.) |
| 1 | D1 | 94.33 | 92.93 | 1 | 58.2 |
| 2 | D2 | 92.34 | 95.97 | 1 | 56.9 |
| 3 | D3 | 174.32 | 98.92 | 1 | 58.2 |
| 4 | D43 | 142.24 | 99.38 | 1 | 57.1 |
| 5 | D44 | 122.88 | 93.01 | 1 | 58.5 |

### a) D1 (Ang-2/MSLN/GITR)

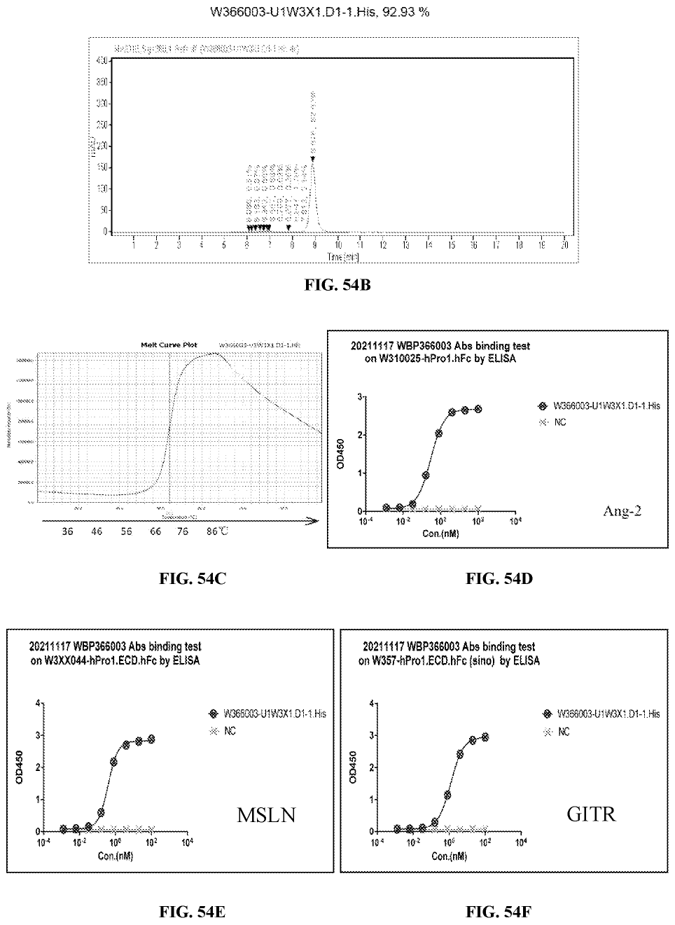

The schematic structure of the multispecific antibody D1 is shown in FIG. 53A. After purification, the purified D1 was analyzed by non-reducing (NR) and reducing (R) gel electrophoresis, respectively. As shown in FIG. 54A, the results indicate that the D1 antibody was successfully purified with correct molecular weight. The SEC result is shown in FIG. 54B, in which a single major peak was observed. The final purity of the D1 antibody was determined as 92.93%. In addition, the Tₘ1 and Tₘ2 values of D1 were determined as 58.2° C. and 66.5° C., respectively, based on the melting curve shown in FIG. 54C.

In addition, the binding capabilities of D1 to Ang-2, Mesothelin, and GITR were measured by ELISA, and the results are shown in FIGS. 54D-54F, respectively. The EC50 values of D1 binding to Ang-2, Mesothelin, and GITR were determined as 0.2955 μg/ml, 0.3934 μg/ml and 1.2280 μg/ml, respectively.

### b) D2 (Ang-2/MSLN/GITR)

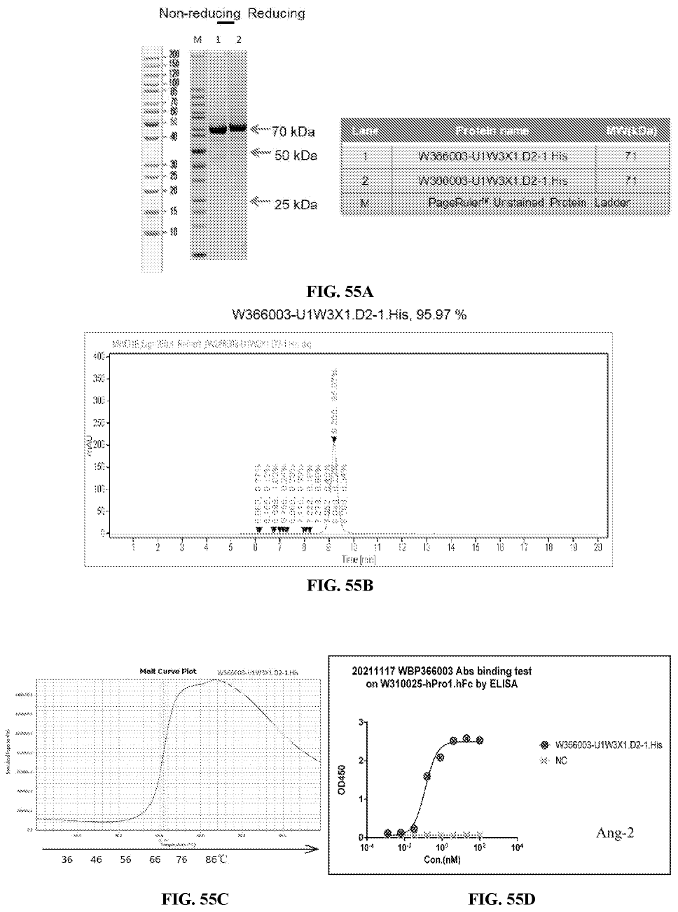

The schematic structure of the multispecific antibody D2 is shown in FIG. 53B. After purification, the purified D2 was analyzed by non-reducing (NR) and reducing (R) gel electrophoresis, respectively. As shown in FIG. 55A, the results indicate that the D2 antibody was successfully purified with correct molecular weight. The SEC result is shown in FIG. 55B, in which a single major peak was observed. The final purity of the D2 antibody was determined as 95.97%. In addition, the Tₘ1 and Tₘ2 values of D2 were determined as 56.9° C. and 67.9° C., respectively, based on the melting curve shown in FIG. 55C.

In addition, the binding capabilities of D2 to Ang-2, Mesothelin, and GITR were measured by ELISA, and the results are shown in FIGS. 55D-55F, respectively. The EC50 values of D2 binding to Ang-2, Mesothelin, and GITR were determined as 0.1269 μg/ml, 0.2724 μg/ml and 0.3624 μg/ml, respectively.

### c) D3 (Ang-2/MSLN/GITR)

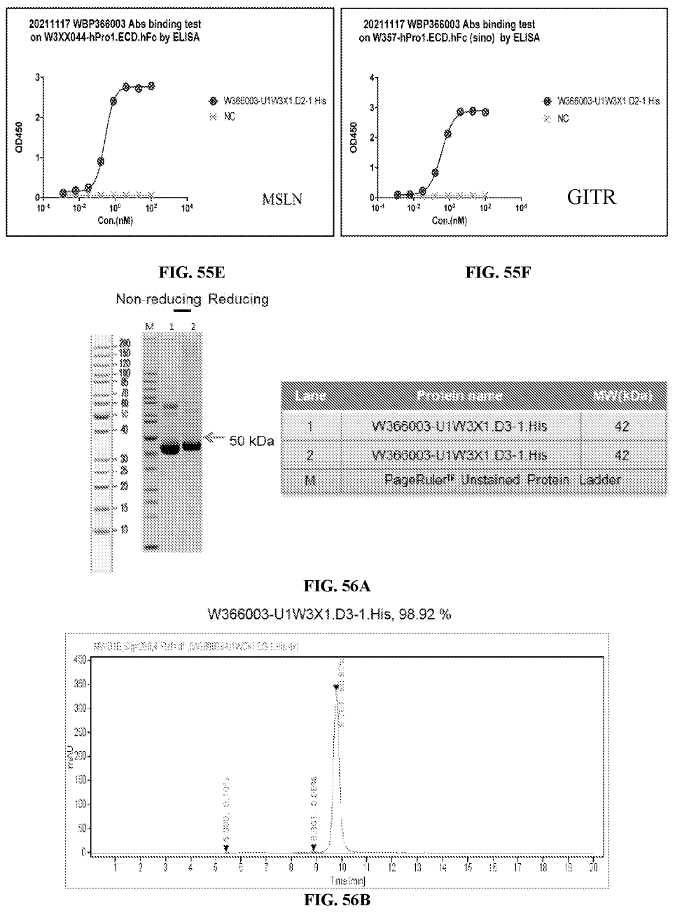

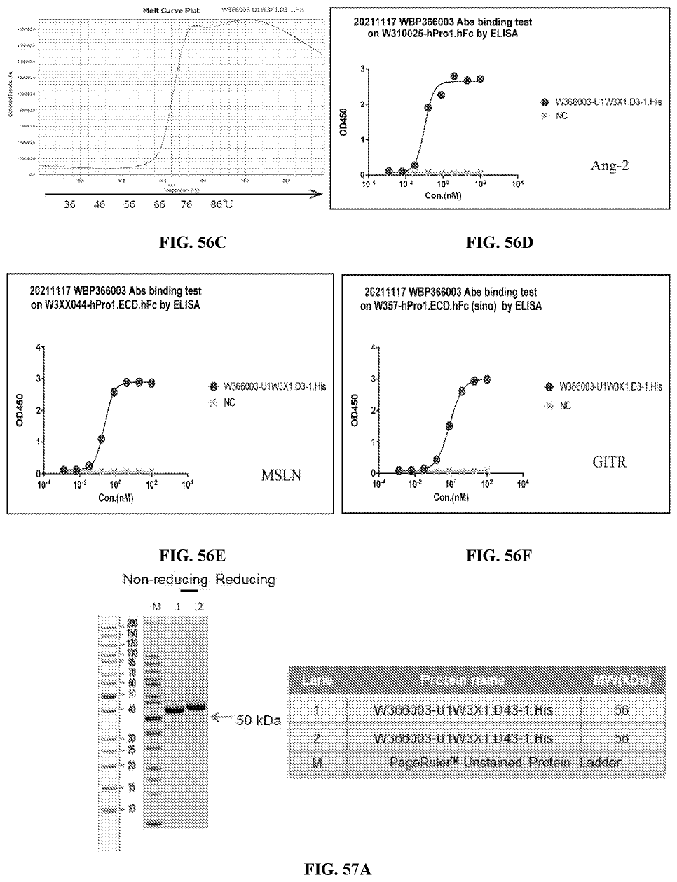

The schematic structure of the multispecific antibody D3 is shown in FIG. 53C. After purification, the purified D3 was analyzed by non-reducing (NR) and reducing (R) gel electrophoresis, respectively. As shown in FIG. 56A, the results indicate that the D3 antibody was successfully purified with correct molecular weight. The SEC result is shown in FIG. 56B, in which a single major peak was observed. The final purity of the D3 antibody was determined as 98.92%. In addition, the Tₘ1 and Tₘ2 values of D3 were determined as 58.2° C. and 72.5° C., respectively, based on the melting curve shown in FIG. 56C.

In addition, the binding capabilities of D3 to Ang-2, Mesothelin, and GITR were measured by ELISA, and the results are shown in FIGS. 56D-56F, respectively. The EC50 values of D3 binding to Ang-2, Mesothelin, and GITR were determined as 0.1036 μg/ml, 0.2305 μg/ml and 0.8347 μg/ml, respectively.

### d) D43 (Ang-2/MSLN/GITR)

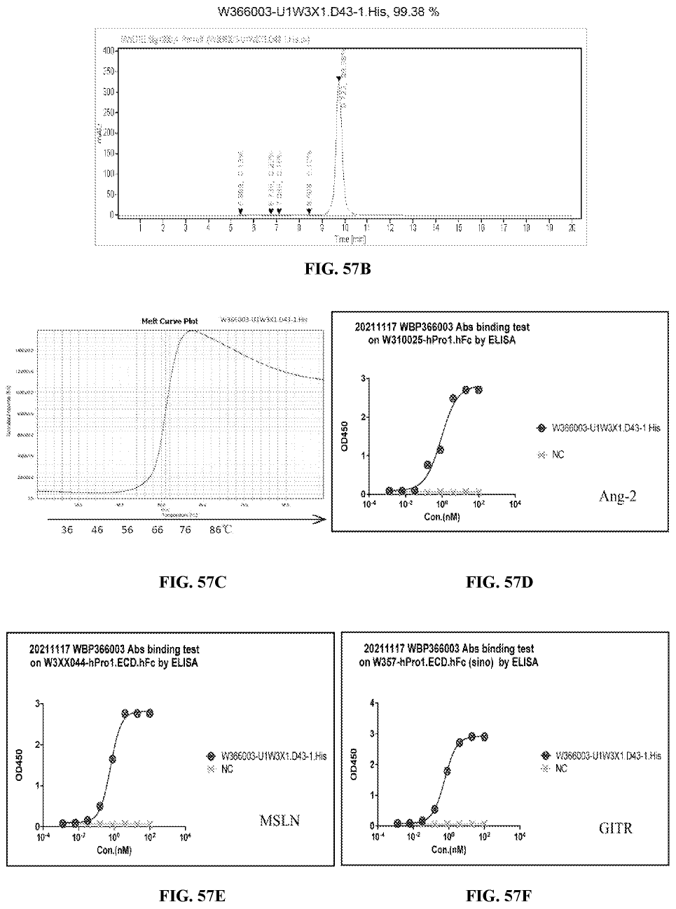

The schematic structure of the multispecific antibody D43 is shown in FIG. 53D. After purification, the purified D43 was analyzed by non-reducing (NR) and reducing (R) gel electrophoresis, respectively. As shown in FIG. 57A, the results indicate that the D43 antibody was successfully purified with correct molecular weight. The SEC result is shown in FIG. 57B, in which a single major peak was observed. The final purity of the D43 antibody was determined as 99.38%. In addition, the Tₘ1 value of D43 was determined as 57.1° C., respectively, based on the melting curve shown in FIG. 57C.

In addition, the binding capabilities of D43 to Ang-2, Mesothelin, and GITR were measured by ELISA, and the results are shown in FIGS. 57D-57F, respectively. The EC50 values of D43 binding to Ang-2, Mesothelin, and GITR were determined as 0.8960 μg/ml, 0.6219 μg/ml and 0.5783 μg/ml, respectively.

### e) D44 (Ang-2/MSLN/GITR)

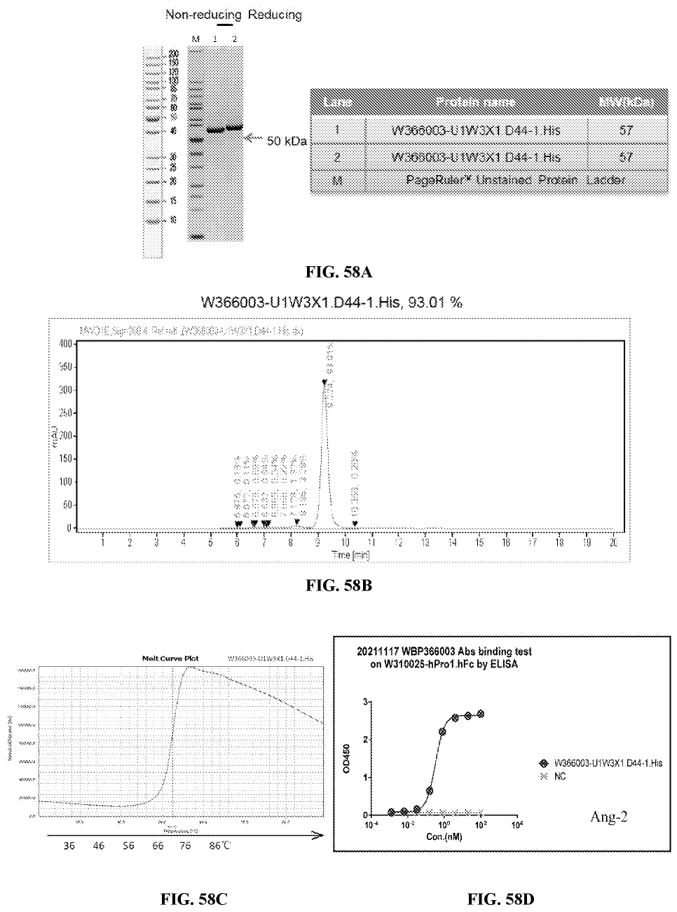

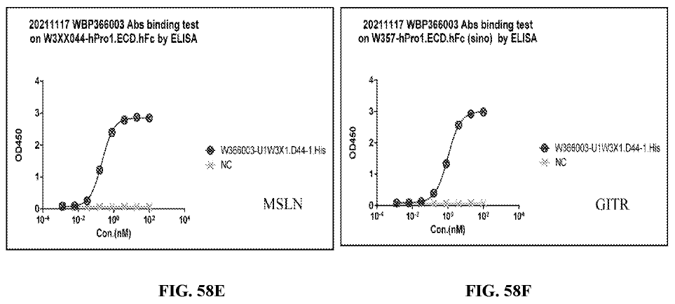

The schematic structure of the multispecific antibody D44 is shown in FIG. 53E. After purification, the purified D44 was analyzed by non-reducing (NR) and reducing (R) gel electrophoresis, respectively. As shown in FIG. 58A, the results indicate that the D44 antibody was successfully purified with correct molecular weight. The SEC result is shown in FIG. 58B, in which a single major peak was observed. The final purity of the D44 antibody was determined as 93.01%. In addition, the Tₘ1 and Tₘ2 values of D44 were determined as 58.5° C. and 67.7° C., respectively, based on the melting curve shown in FIG. 58C.

In addition, the binding capabilities of D44 to Ang-2, Mesothelin, and GITR were measured by ELISA, and the results are shown in FIGS. 58D-58F, respectively. The EC50 values of D44 binding to Ang-2, Mesothelin, and GITR were determined as 0.3288 μg/ml, 0.2144 μg/ml and 0.9958 μg/ml, respectively.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-117. (canceled)

118. An antigen binding protein, comprising
(a) a first antigen binding site comprising a first single-domain antibody variable domain (VHH) that specifically binds to a first epitope; and
(b) a second component comprising a Fc and/or a second antigen binding site comprising a Fab, an scFv or a second VHH that specifically binds to a second epitope;
wherein the first antigen binding site and the second component are linked.

119. The antigen binding protein of claim 118, wherein the second component comprises a Fc, and wherein:
(a) the first VHH is linked to a CH2 domain in the Fc;
(b) the first VHH is linked to the C terminal of a CH3 domain in the Fc; or
(c) the first VHH is linked to a CH1 domain, and the CH1 domain is linked to a CH2 domain in the Fc.

120. The antigen binding protein of claim 118, wherein the second component comprises a Fc, a VH domain and a VL domain, the first VHH is linked to the VH domain and a CH1 domain, and the CH1 domain is linked to a CH2 domain in the Fc, wherein the VH domain and the VL domain associate with each other, forming an antigen binding site.

121. The antigen binding protein of claim 118, wherein the second component comprises a Fc and a second VHH, and wherein:
(a) the second VHH is linked to a CH2 domain in the Fc;
(b) the second VHH is linked to the C terminal of a CH3 domain in the Fc;
(c) the second VHH is linked to a CH1 domain, and the CH1 domain is linked to a CH2 domain in the Fc;
(d) the first VHH is linked to a CH1 domain and the second VHH is linked to a CL domain;
(e) the second component further comprises a VH domain and a VL domain that associate with each other, wherein the second VHH is linked to the VH domain and a CH1 domain and the CH1 domain is linked to a CH2 domain in the Fc, or the second VHH is linked to the VL domain; or
(f) the second component further comprises a VH domain and a VL domain that associate with each other, wherein the second VHH is linked to the VH domain or the VL domain.

122. The antigen binding protein of claim 118, wherein the antigen binding protein further comprises a third antigen-binding site comprising a third VHH that specifically binds to a third epitope, wherein the third VHH is linked to the first VHH, optionally the first epitope and the third epitope are from the same or different antigens.

123. The antigen binding protein of claim 118, wherein the first antigen binding site and the second antigen binding site specifically bind to one or more of the following antigens selected from the group consisting of VEGF, Ang-2, MSLN, GITR, and PD-1, and the first epitope and the second epitope are from the same or different antigens.

124. The antigen binding protein of claim 118, comprising:
(a) a first polypeptide comprising domains linked into the format of VHH2-Fc, a second polypeptide comprising domains linked into the format of VHH1-CH1-Fc and a third polypeptide comprising domains linked into the format of VHH1-CL;
(b) a first polypeptide comprising domains linked into the format of VHH2-CH1-Fc, a second polypeptide comprising domains linked into the format of VHH1-CL;
(c) a first polypeptide comprising domains linked into the format of VHH1-VHH2-Fc, a second polypeptide comprising domains linked into the format of VHH1-CH1-Fc and a third polypeptide comprising domains linked into the format of VHH1-CL;
(d) a first polypeptide comprising domains linked into the format of VHH2-CH1-Fc, a second polypeptide comprising domains linked into the format of VHH1-CH1-Fc and a third polypeptide comprising domains linked into the format of VHH1-CL;
(e) a first polypeptide comprising domains linked into the format of VHH1-CH1-Fc-VHH2, and a second polypeptide comprising domains linked into the format of VHH1-CL;
(f) a first polypeptide comprising domains linked into the format of VHH1-VH-CH1-Fc, and a second polypeptide comprising domains linked into the format of VHH1-VL-CL;
(g) a first polypeptide comprising domains linked into the format of VHH1-VHH2-Fc, a second polypeptide comprising domains linked into the format of VHH3-CH1-Fc and a third polypeptide comprising domains linked into the format of VHH4-CL;
(h) a first polypeptide comprising domains linked into the format of VHH1-VHH2-Fc, and a second polypeptide comprising domains linked into the format of VHH3-VHH4-Fc;
(i) a first polypeptide comprising domains linked into the format of VHH1-VHH2-Fc, a second polypeptide comprising domains linked into the format of VHH3-VH-CH1-Fc and a third polypeptide comprising domains linked into the format of VL-CL; or
(j) a polypeptide comprising domains linked into the format of VHH1-VHH2-Fc-VHH3-VHH4;
wherein VHH2 is the second VHH, VHH3 is a third VHH that specifically binds to a third epitope, VHH4 is a fourth VHH that specifically binds to a fourth epitope, the VH and the VL associate with each other, forming an antigen binding site;
wherein the linkage between two domains is direct or indirect via a peptide linker.

125. A multispecific antigen binding protein, comprising a first polypeptide comprising a first VHH (VHH1) that specifically binds to a first epitope, and a second polypeptide comprising a second VHH (VHH2) that specifically binds to a second epitope, wherein the first polypeptide and the second polypeptide associate with each other to form a dimer, and wherein
(a) the first polypeptide further comprises a first immunoglobulin hinge region, a first CH2 domain and a first CH3 domain, and the second polypeptide further comprises a second immunoglobulin hinge region, a second CH2 domain and a second CH3 domain, the VHH1 is linked to the first immunoglobulin hinge region and the VHH2 is linked to the second immunoglobulin hinge region; or
(b) the first polypeptide further comprises a first CH1 domain, and the second polypeptide further comprises a CL domain, optionally a VH domain is located between the VHH1 and the CH1 domain, and a VL domain is located between the VHH2 and the CL domain, wherein the VH and VL associate with each other, forming an antigen binding site; or
(c) the first polypeptide further comprises a first CH1 domain, and the second polypeptide further comprises a second CH1 domain, the VHH1 is linked to the first CH1 domain and the VHH2 is linked to the second CH1 domain.

126. The antigen binding protein of claim 125, wherein the antigen binding protein of (c) further comprises a third polypeptide, wherein the third polypeptide comprises a third single-domain antibody (VHH3) that specifically binds a third epitope and a first CL domain, wherein the first polypeptide and the third polypeptide associate with each other via the interaction between the first CH1 domain and the first CL domain;
optionally, the antigen binding protein of (c) further comprises a fourth polypeptide which comprises a fourth VHH (VHH4) that specifically binds a fourth epitope and a second CL domain, wherein the second polypeptide and the fourth polypeptide associate with each other via the interaction between the second CH1 domain and the second CL domain;
optionally, the first polypeptide further comprises a fifth VHH (VHH5) that specifically binds to a fifth epitope, wherein the VHH5 is linked to the N-terminus or C-terminus of the first polypeptide;
optionally, the second polypeptide further comprises a sixth VHH (VHH6) that specifically binds to a sixth epitope, wherein the VHH6 is linked to the N-terminus or C-terminus of the second polypeptide;
optionally, the third polypeptide further comprises a seventh VHH (VHH7) that specifically binds to a seventh epitope, wherein the VHH7 is linked to the N-terminus or C-terminus of the third polypeptide;
optionally, the fourth polypeptide further comprises an eighth VHH (VHH8) that specifically binds to an eighth epitope, wherein the VHH8 is linked to the N-terminus or C-terminus of the fourth polypeptide.

127. An antigen binding protein, comprising
a first polypeptide comprising a first VHH (VHH1) that specifically binds to a first epitope; and
a second polypeptide comprising a first heavy chain variable domain (VH1) and a first CH1 domain of a first Fab domain, wherein the first Fab domain specifically binds to a second epitope,
wherein the first polypeptide and the second polypeptide associate with each other to form a dimer.

128. The antigen binding protein of claim 127, wherein the first polypeptide further comprises from N-terminus to C-terminus:
(a) a first immunoglobulin hinge region, a first CH2 domain and a first CH3 domain, wherein the VHH1 is linked to the first immunoglobulin hinge region; or
(b) a second heavy chain variable domain VH (VH2) and a second CH1 domain of a second Fab domain, a first immunoglobulin hinge region, a first CH2 domain and a first CH3 domain,
optionally, the second polypeptide further comprises from N-terminus to C-terminus: a second immunoglobulin hinge region, a second CH2 domain and a second CH3 domain.

129. The antigen binding protein of claim 128, wherein in the antigen binding protein of (b), the VHH1 is linked to N-terminus of the VH2 or the VHH1 is located between the second CH1 domain and the first immunoglobulin hinge region.

130. The antigen binding protein of claim 128, wherein the antigen binding protein of (b) further comprises a second VHH (VHH2), wherein the VHH2 is linked to a second light chain variable domain (VL2) of the second Fab domain;
optionally, the antigen binding protein further comprises a third VHH (VHH3), wherein the VHH3 is linked to the N-terminus of the VH1 or located between the first CH1 domain and the second immunoglobulin hinge region;
optionally, the antigen binding protein further comprises a fourth VHH (VHH4), wherein the VHH4 is linked to a first light chain variable domain (VL1) of the first Fab domain;
optionally, the antigen binding protein further comprises a fifth VHH (VHH5) that specifically binds to a fifth epitope, wherein the VHH5 is linked to the N-terminus or C-terminus of the first polypeptide;
optionally, the second polypeptide further comprises a sixth VHH (VHH6) that specifically binds to a sixth epitope, wherein the VHH6 is linked to the N-terminus or C-terminus of the second polypeptide.

131. The antigen binding protein of claim 127, wherein the VHH1, VHH2, VHH3, VHH4, VHH5, VHH6, VHH7, and/or VHH8, if present, specifically bind to a cancer associated antigen or a cancer specific antigen or an immune checkpoint molecule.

132. The antigen binding protein of claim 127, wherein the VHH1, VHH2, VHH3, VHH4, VHH5, VHH6, VHH7, and/or VHH8, if present, specifically bind to an antigen, wherein the antigen is selected from the group consisting of VEGF, Ang2, Mesothelin, GITR, HER2, BRAF, EGFR, VEGFR2, CD20, RANKL, CD38, CD52, PD-1, PD-L1, PD-L2, CTLA-4, B7-H3, TIM-3, LAG-3, VISTA, ICOS, 4-1BB, OX40, GITR, and CD40.

133. A method of treating a subject having a cancer, an autoimmune disease or an inflammatory disease, the method comprising administering a therapeutically effective amount of a composition comprising the antigen binding protein of claim 118 to the subject.

134. The method of claim 133, wherein the subject has a VEGF-expressing, Ang-2-expressing, and/or MSLN-expressing cancer.

135. The method of claim 133, wherein the cancer is selected from the group consisting of breast cancer, renal cancer, melanoma, lung cancer, glioblastoma, head and neck cancer, prostate cancer, ovarian carcinoma, bladder carcinoma, and lymphoma.

136. An antibody-drug conjugate comprising the antigen binding protein of claim 118 covalently bound to a therapeutic agent.

137. A pharmaceutical composition comprising the antigen binding protein of claim 118 and a pharmaceutically acceptor carrier.

138. A nucleic acid encoding the antigen binding protein of claim 118.

139. A vector comprising the nucleic acid of claim 138.

140. A host cell comprising the nucleic acid of claim 138.

141. A method for producing an antigen binding protein, the method comprising culturing the host cell of claim 140 under conditions suitable to produce the antigen binding protein.


SEQUENCE LISTING (6 sequences)

SEQ ID NO: 1 - protein, 5 aa, Artificial sequence
  linker
GSGGS

SEQ ID NO: 2 - protein, 4 aa, Artificial sequence
  linker
GGGS

SEQ ID NO: 3 - protein, 5 aa, Artificial sequence
  linker
GGGGS

SEQ ID NO: 4 - protein, 10 aa, Artificial sequence
  linker
GGGGSGGGGS

SEQ ID NO: 5 - protein, 15 aa, Artificial sequence
  linker
EPKSCDKTHTCPPCP

SEQ ID NO: 6 - protein, 15 aa, Artificial sequence
  linker
EPKSSDKTHTSPPSP